SEQUENCE LISTING (42 sequences)

SEQ ID NO: 1 - DNA, 987 bp, synthetic construct
  source 1..987: mol_type = other DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttag

SEQ ID NO: 2 - DNA, 762 bp, synthetic construct
  source 1..762: mol_type = other DNA
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 3 - DNA, 518 bp, synthetic construct
  source 1..518: mol_type = other DNA
accaggataagatcgaggctctgtcctccaaggtgcagcagctggaacggtccatcggcctgaaggacctggccatggctgacctggaacagaaagtgctggaaatggaagcctccacacaggtatcacatcggtatcctcgaatccaaagtatcaaagtacaatttaccgaatataagaaggagaaaggtttcatcctcacttcccaaaaggaggatgaaatcatgaaggtgcagaacaactcagtcatcatcaactgtgatgggttttatctcatctccctgaagggctacttctcccaggaagtcaacattagccttcattaccagaaggatgaggagcccctcttccaactgaagaaggtcaggtctgtcaactccttgatggtggcctctctgacttacaaagacaaagtctacttgaatgtgaccactgacaatacctccctggatgacttccatgtgaatggcggagaactgattcttatccatcaaaatcctggtgaattttgtgtcctt

SEQ ID NO: 4 - DNA, 333 bp, synthetic construct
  source 1..333: mol_type = other DNA
caggtgcagctggtggagagcggcggcggcgtggtgcagcccggcaggagcctgaggctggactgcaaggccagcggcatcaccttcagcaacagcggcatgcactgggtgaggcaggcccccggcaagggcctggagtgggtggccgtgatctggtacgacggcagcaagaggtactacgccgacagcgtgaagggcaggttcaccatcagcagggacaacagcaagaacaccctgttcctgcagatgaacagcctgagggccgaggacaccgccgtgtactactgcgccaccaacgacgactactggggccagggcaccctggtgaccgtg

SEQ ID NO: 5 - DNA, 315 bp, synthetic construct
  source 1..315: mol_type = other DNA
gagatcgtgctgacccagagccccgccaccctgagcctgagccccggcgagagggccaccctgagctgcagggccagccagagcgtgagcagctacctggcctggtaccagcagaagcccggccaggcccccaggctgctgatctacgacgccagcaacagggccaccggcatccccgccaggttcagcggcagcggcagcggcaccgacttcaccctgaccatcagcagcctggagcccgaggacttcgccgtgtactactgccagcagagcagcaactggcccaggaccttcggccagggcaccaaggtggag

SEQ ID NO: 6 - DNA, 508 bp, synthetic construct
  source 1..508: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 7 - DNA, 135 bp, synthetic construct
  source 1..135: mol_type = other DNA
gatccagacatgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 8 - DNA, 1419 bp, synthetic construct
  source 1..1419: mol_type = other DNA
tccacgtcgaaggcttttgcattgtaaagctacccgcctacccgcgcctcccaataaaaaaagaacatacaccaatgggtcttatttggtattacctggtttatttaaaaagatatacagtaagacatcccatggtaccaaagaccggggcgaatcagcgggcccccatcatctgagagacgaacaaatcggcggcgcgggccgtgtcaacgtccacgtgtgctgcgctgctggcgttgacaagggccccggcctccgcgttggatgcctccggttgggatccggtggcggcgggggggaacgcgggctccgtcggtagaggggcgcgcgtctgggtggaaggacatgggggcggtggcgggcctggcgggcaggcagctggggcatacgggggagtgggggcatgggacgccggaccctggggaggaccgtagggggcgctgtgtggtggggggcgatacacggcctccgggggacaaagggccgggtgggtcgttgttggttccggctcccccacctgagggcgatagtgcgccaccggcgtgtacattccatagggggcgctggtccgagcccgcatgtgcgccagttcctgctgcagagacgtcaccgcccccatcagcgccgtgatggtctcgttggtcccgggagactggcgggccgcgcgccgagagtcgaccccgcgcggcccgcctcgagcctccccggggtagtacgggtagtccgcgtccggttcgtcctggtcgcagtaggactccgacggccccgcctcgtaccggcgacgctttcccgacccccggaccccagggtctcccgcggccgtctgaccgcccgcctggcggtccgcggctatggcccccaccaacgcggctatctgcgcctcgagtgggctgggtcccgagaacagcaccccgggatactgatgggcgacgtggggagggtaatgctgggaaagacccgcgccgtgaggcccataggccacggcccccgccgcagccgggaaaccaaacgcggaatgcggctggggttggggcgcggcgtggccggcgacgagctggttgtaatgggaggccgggatccacaggtagctcccgtcgccgggcggcgcgggggccggggtgcccgatgccggaacggggttcatggggggcagtaccgggggcgaggcgaccccgcgctgacggacgattgggcaccggtcaccctgcggcgcggcagcgatcgagccgggcggtatgtccgtggactccggggccccgttcttatacccgcgcgccggcgcggaaacaggctccgccccccacattttgaatttttcgctcgcctggaggtaggtgtgtccggcgatcccggcctgccgccgccgctcggccaccaggctccagcggtcccgcagcatcatgttgttaacggcggtgg

SEQ ID NO: 9 - DNA, 1340 bp, synthetic construct
  source 1..1340: mol_type = other DNA
ggtgcgagctgcagaatcacgagctgaccctgtggaacgaggcccgcaagctgaaccccaacgccatcgcctcggccaccgtgggccggcgggtgagcgcgcggatgctcggcgacgtgatggccgtctccacgtgcgtgccggtcgccgcggacaacgtgatcgtccaaaactcgatgcgcatcagctcgcggcccggggcctgctacagccgccccctggtcagctttcggtacgaagaccagggcccgttggtcgaggggcagctgggggagaacaacgagctgcggctgacgcgcgatgcgatcgagccgtgcaccgtgggacaccggcgctacttcaccttcggcgggggctacgtgtacttcgaggagtacgcgtactcccaccagctgagccgcgccgacatcaccaccgtcagcaccttcatcgacctcaacatcaccatgctggaggatcacgagtttgtccccctggaggtgtacacccgccacgagatcaaggacagcggcctgctggactacacggaggtccagcgccgcaaccagctgcacgacctgcgcttcgccgacatcgacacggtcatccacgccgacgccaacgccgccatgttcgcgggcctgggcgcgttcttcgaggggatgggcgacctggggcgcgcggtcggcaaggtggtgatgggcatcgtgggcggcgtggtatcggccgtgtcgggcgtgtcctccttcatgtccaacccctttggggcgctggccgtgggtctgttggtcctggccggcctggcggcggctttcttcgcctttcgctacgtcatgcggctgcagagcaaccccatgaaggccctgtacccgctaaccaccaaggagctcaagaaccccaccaacccggacgcgtccggggagggcgaggagggcggcgactttgacgaggccaagctagccgaggcccgggagatgatacggtacatggccctggtgtctgccatggagcgcacggaacacaaggccaagaagaagggcacgagcgcgctgctcagcgccaaggtcaccgacatggtcatgcgcaagcgccgcaacaccaactacacccaagttcccaacaaagacggtgacgccgacgaggacgacctgtgacggggggtttgttgtaaataaaaaccacgggtgttaaaccgcatgtgcatcttttggtttgtttgtttggtacgccttttgtgtgtgtgggaagaaagaaaagggaacacataaactcccccgggtgtccgcggcctgtttcctctttcctttcccgtgacaaaacggacccccttggtcagtgccgattccccccccacgccttcc

SEQ ID NO: 10 - DNA, 684 bp, synthetic construct
  source 1..684: mol_type = other DNA
agcccgggccccccgcgggcggggcggcgcgcaaaaaaggcgggcggcggtccgggcggcgtgcgcgcgcgcggcgggcgttggggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggaaaacgggccccccccgaaacacaccccccgggggtcgcgcgcggccctttaaagcgcggcggcgcagcccgggccccccgcggccgagacgagcgagttagacaggcaagcactactcgcctctgcacgcacatgcttgcctgtcaaactctaccaccccggcacgctctctgtctccatggcccgccgccgccgccatcgcggcccccgccgcccccggccgcccgggcccacgggcgccgtcccaaccgcacagtcccaggtaacctccacgcccaactcggaacccgcggtcaggagcgcgcccgcggccgccccgccgccgccccccgccagtgggcccccgccttcttgttcgctgctgctgcgccagtgg

SEQ ID NO: 11 - DNA, 967 bp, synthetic construct
  source 1..967: mol_type = other DNA
gacagccccccgcccgagccggcgccagaggcccggcccaccgccgccgccccccgcccccggtccccaccgcccggcgcgggcccggggggcggggctaacccctcccaccccccctcacgccccttccgccttccgccgcgcctcgccctccgcctgcgcgtcaccgcagagcacctggcgcgcctgcgcctgcgacgcgcgggcggggagggggcgccggagccccccgcgacccccgcgacccccgcgacccccgcgacccccgcgacccccgcgcgggtgcgcttctcgccccacgtccgggtgcgccacctggtggtctgggcctcggccgcccgcctggcgcgccgcggctcgtgggcccgcgagcgggccgaccgggctcggttccggcgccgggtggcggaggccgaggcggtcatcgggccgtgcctggggcccgaggcccgtgcccgggccctggcccgcggagccggcccggcgaactcggtctaacgttacacccgaggcggcctgggtcttccgcggagctcccgggagctccgcaccaagccgctctccggagagacgatggcaggagccgcgcatatatacgctgggagccggcccgcccccgaggcgggcccgccctcggagggcgggactggccaatcggcggccgccagcgcggcggggcccggccaaccagcgtccgccgagtcttcggggcccggcccactgggcgggagttaccgcccagtgggccgggccgcccacttcccggtatggtaattaaaaacttacaagaggccttgttccgcttcccggtatggtaattagaaactcattaatgggcggccccggccgcccttcccgcttccggcaattcccgcggcccttaatgggcaaccccggtattccccgcctcccgcgccgcgcgtaaccactcccctggggttccgggttatgctaattgcttttttgg

SEQ ID NO: 12 - DNA, 1237 bp, synthetic construct
  source 1..1237: mol_type = other DNA
cgttcggacgtcttagaatcatggcggttttctatgccgacatcggttttctcccccgcaataagacacgatgcgataaaatctgtttgtgaaatttattaagggtacaaattgccctagcacaggggtggggttagggccgggtccccacacccaaacgcaccaaacagatgcaggcagtgggtcgagtacagccccgcgtacgaacacgtcgatgcgtgtgtcagacagcaccagaaagcacaggccatcaacaggtcgtgcatatgtcggtgggtttggacgcggggggccatggtggtgataaagttaatggccgccgtccgccagggccacaggggcgacgtctcttggttggcccggagccactgggtgtggaccagccgcgcgtggcggcccaacatggcccctgtagccgggggcgggggatcgcgcacgtttgcagcgcacatgcgagacacctcgaccacggttcggaagaaggcccggtggtccgcgggcaacatcaccaggtgcgcaagcgcccgggcgtccagagggtagagccctgagtcatccgaggttggctcatcgcccgggtcatgccgcaagtgcgtgtgggttgggcttccggtgggcgggacgcgaaccgcggtgtggagccctacgcgggcccgagcgtacgctccatcttgtggggagaaggggtctgggctcgccaggggggcatacttgcccgggctatacagacccgcgagccgtacgtggttcgcggggggtgcgtggggtccggggctcccggggaggccggggctcccggggttgtcgtggatccctggggtcacgcggtaccctggggtctctgggagctcgcggtactctgggttccctaggttctcggggtggtcgcggaacccggggctcccggggaacacgcggtgtcctggggattgttggcggtcggacggcttcagatggcttcgagatcgtagtgtccgcaccgactcgtagtagacccgaatctccacattgccccgccgcttgatcattatcaccccgttgcgggggtccggagatcatgcgcgggtgtcctcgaggtgcgtgaacacctctggggtgcatgccggcggacggcacgccttttaagtaaacatctgggtcgcccggcccaactggggccgggggttgggtctggctcatctcgagagccacggggggaaccaccctccgcccagaaacttgggcgatggtcgtaccc

SEQ ID NO: 13 - DNA, 1238 bp, synthetic construct
  source 1..1238: mol_type = other DNA
gcctcgacgaggacgttcctcctgcgggaaggcacgaacgcgggtgagccccctcctccgcccccgcgtcccccctcctccgcccccgcgtcccccctcctccgcccccgcgtcccccctcctccgcccccgcgtcccccctcctccgcccccgcgtcccccctcctccgcccccgcgtcccccctcctccacccccgcgtccccccctcctccgcccacccaaggtgcttacccgtgcacaaaggcggaccggtgggtttctgtcgtcggaggcccccggggtgcgtcccctgtgtttcgtgggtggggtgggtgggtctttccgcgtgtccctttccgatgcgatcccgatcccgagccggggcgtcgcgatgccgacgccgtccgctccgacggccctctgcgagtcccgctcccggtccgcgtgctccgcagccgctcccgtcgttcgtggccggcgccgtctgcgggcgtcggtcgcgccgggcctttatgtgcgccggagagacccgccccccgccgcccgggcccgcccccggggccggcgcggagtcgggcacggcgccagtgctcgcacttcgccctaataatatatatatattgggacgaagtgcgaacgcttcgcgttctcacttcttttacccggcggccccgcccccttggggcggtcccgcccgccggccaatgggggggcggcaaggcgggcggcccttgggccgcccgccgtcccgttggtcccaacgtccggcgggcgggaccgggggcccggggacggccaacgggcgcgcggggctcgtatctcattaccgccgaaccgggaagtcggggcccgggccccgcccccggcccgttcctcgttagcatgcggaacggaagcggaaaccaccggatcgggcggtaatgagatgccatgcggggcggggcgcgggcccacccgccctcgcgccccgcccatggcagatggcgcggatgggcggggccgggggttcgaccaacgggccgcggccacgggcccccggcgtgccggcgtcggggcggggtcgtgcataatggaattccgttcggggcgggcccgcctggggggcggggggccggcggcctccgctgctcctccttcccgccggcccctgggactatatgagcccgaggacgccccgatcgtccacacggagcgcggctgccgacacggatccacgacccgacgcgggaccgccagagacagaccgtcagacgctcg

SEQ ID NO: 14 - DNA, 1113 bp, synthetic construct
  source 1..1113: mol_type = other DNA
gttactaaacacgaccctgaccgtcaagcgcggggcggcggcgtcccactctagaatcggttgggaccgcttcgtgggcggagttatccgccggttggccgcgcgccgccccggcctggtgtttatgctctggggcgcacatgcccagaatgccatcaggccggaccctcgggtccattgcgtcctcaagttttcgcacccgtcgcccctctccaaggttccgttcggaacatgccagcatttcctcgtggcgaatcgatatctcgagacccggtcgatttcacccatcgactggtcggtttgaaaggcatcgacgtccggggttttcgtctgtgggggcttttgggtatttccgatgaataaagacggttaatggttaaacctctggtctcatacgggtcggtgatgtcgggcgtcgggggagagggagttccctctgcgcttgcgattctagcctcgtggggctggacgttcgacacgccaaaccacgagtcagggatatcgccagatacgactcccgcagattccattcggggggccgctgtggcctcacctgaccaacctttacacgggggcccggaacgggaggccacagcgccgtctttctccccaacgcgcgcggatgacggcccgccctgtaccgacgggccctacgtgacgtttgataccctgtttatggtgtcgtcgatcgacgaattagggcgtcgccagctcacggacaccatccgcaaggacctgcggttgtcgctggccaagtttagcattgcgtgcaccaagacctcctcgttttcgggaaacgccccgcgccaccacagacgcggggcgttccagcgcggcacgcgggcgccgcgcagcaacaaaagccttcagatgtttgtgttgtgcaaacgcacccacgccgctcgagtgcgagagcagcttcgggtcgttattcagtcccgcaagccgcgcaagtattacacgcgatcttcggacgggcggctctgccccgccgtccccgtgttcgtccacgagttcgtctcgtccgagccaatgcgcctccaccgagataacgtcatgctggcctcgggggccgagtaaccgcccccccgcgccaccctcactgcccgtcgc

SEQ ID NO: 15 - DNA, 1031 bp, synthetic construct
  source 1..1031: mol_type = other DNA
gcgtgtttgatgttaataaataacgcataaatttggctggttgtttgttgtctttaatggaccgcccgcagggggggtggcatttcagtgtcgggtgacgagcgcgatccggccgggatcctaggaccccaaaagtttgtctgcgtattccagggcggggctcagttgaatctcccgcagcacctctaccagcaggtccgcggtgggctggagaaactcggccgtcccggggcaggcggtcgtcgggggtggaggcgcggcgcccaccccgtgtgccgcgcctggcgtctcctctgggggcgacccgtaaatggttgcagtgatgtaaatggtgtccgcggtccagaccacggtcaaaatgccggccgtggcgctccgggcgctttcgccgcgcgaggagctgacccaggagtcgaacggatacgcgtacatatgggcgtcccacccgcgttcgagcttctggttgctgtcccggcctataaagcggtaggcacaaaattcggcgcgacagtcgataatcaccaacagcccaatgggggtgtgttggataacaacgcctccgcgcggcaggcggtcctggcgctcccggccccgtaccatgatcgcgcgggtgccgtactcaaaaacatgcaccacctgcgcggcgtcgggcagtgcgctggtcagcgaggccctggcgtggcataggctatacgcgatggtcgtctgtggattggacatctcgcggtgggtagtgagtcccccgggccgggttcggtggaactgtaaggggacggcgggttaatatacaatgaccacgttcggatcgcgcagagccgatagtatgtgcttactaatgacgtcatcgcgctcgtggcgctcccggagcggatttaagttcatgcgaaggaattcggaggaggtggtgcgggacatggccacgtacgcgctgttgaggcgcaggttgccgggcgtaaagcagatggcgaccttgtccaggctaaggccctgggagcgcgtgatggtcatggcaagcttggagctgatgccgt

SEQ ID NO: 16 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 17 - protein, 257 aa, synthetic construct
MATTMWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 18 - protein, 309 aa, synthetic construct
MLPLLLGLYGSVTSALKAAELYPRAHRIMYRMQLLSCIALSLALVTNSDQDKIEALSSKVQQLERSIGLKDLAMADLEQKVLEMEASTQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLASVDYKDDDDKGSTSGSGKPGSGEGSTKGENLYFQGDLNAVGQDTQEVIVVPHSLPFKVVVISAILALVVLTIISLIILIMLWQKKPR

SEQ ID NO: 19 - protein, 253 aa, synthetic construct
MYRMQLLSCIALSLALVTNSQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVE

SEQ ID NO: 20 - protein, 113 aa, synthetic construct [sequence omitted]

SEQ ID NO: 21 - protein, 105 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVE

SEQ ID NO: 22 - protein, 5 aa, synthetic construct
NSGMH

SEQ ID NO: 23 - protein, 17 aa, synthetic construct
VIWYDGSKRYYADSVKG

SEQ ID NO: 24 - protein, 4 aa, synthetic construct
NDDY

SEQ ID NO: 25 - protein, 11 aa, synthetic construct
RASQSVSSYLA

SEQ ID NO: 26 - protein, 7 aa, synthetic construct
DASNRAT

SEQ ID NO: 27 - protein, 9 aa, synthetic construct
QQSSNWPRT

SEQ ID NO: 28 - protein, 20 aa, synthetic construct
MYRMQLLSCIALSLALVTNS

SEQ ID NO: 29 - DNA, 19 bp, synthetic construct
  source 1..19: mol_type = other DNA
tgttctaaagtccgcacgc

SEQ ID NO: 30 - DNA, 19 bp, synthetic construct
  source 1..19: mol_type = other DNA
tacctcatggagtgggagc

SEQ ID NO: 31 - DNA, 20 bp, synthetic construct
  source 1..20: mol_type = other DNA
cagttcgtgcggttcgacag

SEQ ID NO: 32 - DNA, 20 bp, synthetic construct
  source 1..20: mol_type = other DNA
gcctggcgcttgtacttctg

SEQ ID NO: 33 - DNA, 20 bp, synthetic construct
  source 1..20: mol_type = other DNA
tggtcctgtcctgttctcca

SEQ ID NO: 34 - DNA, 20 bp, synthetic construct
  source 1..20: mol_type = other DNA
agaaacgtggtctggtgtcc

SEQ ID NO: 35 - DNA, 20 bp, synthetic construct
  source 1..20: mol_type = other DNA
ttgctgaacgccccatacaa

SEQ ID NO: 36 - DNA, 20 bp, synthetic construct
  source 1..20: mol_type = other DNA
tccagatgacttcggccttg

SEQ ID NO: 37 - DNA, 23 bp, synthetic construct
  source 1..23: mol_type = other DNA
ctcagaagtggagtcttaccctg

SEQ ID NO: 38 - DNA, 20 bp, synthetic construct
  source 1..20: mol_type = other DNA
tgttcctgggtctccaaagg

SEQ ID NO: 39 - DNA, 20 bp, synthetic construct
  source 1..20: mol_type = other DNA
cgcccacttgtcccactatc

SEQ ID NO: 40 - DNA, 20 bp, synthetic construct
  source 1..20: mol_type = other DNA
cattagcccatgcaacagcc

SEQ ID NO: 41 - DNA, 21 bp, synthetic construct
  source 1..21: mol_type = other DNA
tagcacagacacacggatgag

SEQ ID NO: 42 - DNA, 22 bp, synthetic construct
  source 1..22: mol_type = other DNA
actgaagttagcagagagcagg

(19) United States
(12) Patent Application Publication
(10) Pub. No.: US 2025/0319144 A1
(43) Pub. Date: Oct. 16, 2025

(54) Recombinant Armed Oncolytic Virus Composition and Use Thereof in TIL Adoptive Therapy

(71) Applicants: Nankai University, Tianjin (CN); Beijing Biological Products Institute Co., Ltd., Beijing (CN)

(72) Inventors: Hongkai ZHANG, Tianjin (CN); Yuntao ZHANG, Tianjin (CN); Kai YE, Tianjin (CN); Li DENG, Tianjin (CN); Fan LI, Tianjin (CN); Wenrui GAO, Tianjin (CN); Tianyi CEN, Tianjin (CN); Miaomiao GUO, Tianjin (CN); Lili XU, Tianjin (CN)

(21) Appl. No.: 18/711,304

(22) PCT Filed: Nov. 18, 2022
(86) PCT No.: PCT/CN2022/132915

§ 371 Date: Dec. 13, 2024

(30) Foreign Application Priority Data
Nov. 19, 2021 (CN) 202111391666.0

(51) Int. Cl. A61K 35/763; A61K 38/00; A61P 35/00; C07K 14/54; C07K 14/705; C07K 16/28; C12N 7/00

(52) U.S. Cl. CPC A61K 35/763; A61P 35/00; C07K 14/5434; C07K 14/70575; C07K 16/2818; C12N 7/00; A61K 38/00; C07K 2317/565; C07K 2317/622; C12N 2710/16621; C12N 2710/16632

(57) ABSTRACT

A recombinant armed oncolytic virus composition for conversion of tumor cells into APCs, specifically herpes simplex oncolytic virus composition. The oncolytic virus composition infects tumor cells and expresses trimeric OX40L and IL-12 and optionally a PD-1 inhibitor. Also provided is the use of the oncolytic virus composition for enhancing antigen presentation of tumor cells, and for enhancing the anti-tumor effect of a tumor infiltrating lymphocyte (TIL) in cancer therapy. Further provided are a pharmaceutical composition, a kit, and a combination product for the methods and uses.

### TECHNICAL FIELD

The present invention relates to the field of cancer treatment. More specifically, the present invention provides recombinant armed oncolytic virus compositions useful in conversion of tumor cells into APCs, particularly herpes simplex oncolytic virus compositions, wherein the oncolytic virus compositions infect tumor cells and express trimeric OX40L and IL-12 and optionally a PD-1 inhibitor. The present invention also provides the use of the oncolytic virus compositions in cancer treatment for enhancing antigen presentation of tumor cells, and for enhancing the anti-tumor efficacy of tumor infiltrating lymphocytes (TILs). The present invention further provides pharmaceutical compositions, kits and combination products for the said methods and uses.

### BACKGROUND TECHNOLOGY

The anti-tumor immune response that eliminates tumors generally involves two phases: i) the induction phase, where naive anti-tumor T cell responses are initiated; and ii) the effector phase, where the induced anti-tumor T cells destroy and clear the tumor. During the induction phase of the anti-tumor response, professional APCs expressing MHC-I and II molecules as well as co-stimulatory molecules (e.g. CD80 and CD86) present antigens to naive T cells. Activation of anti-tumor T cells typically requires at least two signals: i) signal one, induced by the interaction of the MHC/antigen complex with the T cell receptor (TCR), transmitting an activation signal to the T cell; and ii) signal two, induced by the interaction of the co-stimulatory molecules CD80/CD86 with the stimulatory receptor CD28 expressed on the T cell. These two signals lead to activation of CD4 T cells (through MHC-II) and CD8 T cells (through MHC-I). When there is signal one but no signal two, T cell anergy occurs.

Downregulation of antigen presentation is a major mechanism of tumor immune evasion, allowing tumor cells to escape recognition and destruction by anti-tumor T cells. Tumor cells can reduce antigen presentation by several mechanisms: (1) loss of tumor antigens; (2) downregulation or mutation of MHC genes leading to low or no MHC molecule expression; (3) alteration of the antigen loading on MHC; and (4) downregulation of the co-stimulatory molecules CD80 and CD86 to prevent signaling from MHC to T cells.

Current strategies for enhancing tumor antigen presentation to augment the induction phase of the anti-tumor response involve dendritic cell (DC)-based interventions (i.e. loading DCs with tumor antigens in vivo or ex vivo), peptide or DNA vaccines, or TLR agonists. Most of these approaches require the presence of functional DCs. However, DCs are often deficient or tolerogenic in cancer patients, limiting the potential efficacy of such approaches.

It has been proposed that forcing tumor cells themselves to regain or acquire antigen presentation capabilities could be an alternative to DC-based immunotherapy approaches.

Ostrand-Rosenberg S. et al. have demonstrated in pre-clinical mouse models that cancer cells, which acquire APC properties through transfection to express MHC class I and II molecules and the co-stimulatory molecules CD80 and CD86, can effectively present their own antigens, activate immune responses and promote infiltration and tumor clearance by lymphocytes (Ostrand-Rosenberg S. Tumor immunotherapy: the tumor cell as an antigen-presenting cell. Curr Opin Immunol 1994; 6: 722e7.)

The study by Tanaka et al. (Reversal of oncogenesis by the expression of a major histocompatibility complex class I gene. Science 1985:228.) showed that mice bearing MHC class I-transfected tumor cells survived longer than those bearing the parental MHC class I-negative tumor cells, suggesting that restoration of MHC-I expression improves recognition of tumor cells by cytotoxic CD8+ T cells.

Another study by Ostrand-Rosenberg S. et al. (Ostrand-Rosenberg S, Takur A, Clements V. Rejection of mouse sarcoma cells after transfection of MHC class II genes. J Immunol 1990: 4068e71.) showed that highly malignant sarcoma cells after transfection of MHC class II genes exhibited stunted tumor growth in mice, although injection of MHC-II transfected sarcoma cells into immunocompromised mice led to tumor growth. This suggests that MHC-II transfected tumor cells are immunogenic and can be rejected by the immune system. Therefore, it has been proposed that restoring/inducing MHC-II expression in cancer cells could activate CD4+ helper T cells, enhance the cytolytic activity of CD8+ cytotoxic T cells, and enable cytotoxic CD4+ T cells to eliminate tumor cells.

In the T cell activation process, expression of the co-stimulatory molecules CD80/CD86 on cancer cells is required to deliver the second signal (through CD28) to T cells to initiate an anti-tumor response and prevent T cell anergy. Multiple studies have shown that forced expression of CD80/CD86 in tumor cells expressing MHC class I and II, such as through gene transfection, can promote tumor rejection and establish long-term immunity. See e.g. Chen L et al. Costimulation of antitumor immunity by the B7 counterreceptor for the T lymphocyte molecules CD28 and CTLA-4. Cell 1992; 71: 1093e102; and Townsend SE, Allison JP. Tumor rejection after direct co-stimulation of CD8 T cells by B7-transfected melanoma cells. Science 1993; 259: 368e70.

However, these approaches, which rely on viral vector-mediated transfection to express MHC class I and II and/or co-stimulatory molecules to restore antigen presentation in tumor cells and enhance their recognition by antigen-specific TILs, remain limited in their application due to several factors, including tumor tissue heterogeneity and the diverse defective mechanisms responsible for the downregulation of antigen presentation.

Tumor-infiltrating lymphocytes (TILs) are tumor-specific immune cells that naturally occur within tumors. They are recruited by the immune system and infiltrate tumor tissues during an in vivo immune response, and they exhibit cytotoxic activity specifically against tumor cells. TIL cell therapy utilizes these endogenous TILs to suppress or destroy tumors. TIL therapy generally involves isolating TILs from a patient's tumor tissue, expanding them ex vivo to sufficient numbers, and then re-infusing them into the patient. As TILs have inherent cytotoxic effects on solid tumors, adoptive TIL therapy has been proposed for various solid tumors. However, due to the low level of immune infiltration and the scarcity of antigen-presenting cells in most solid tumors, the indications for and efficacy of current TIL therapy are highly limited, leaving the majority of patients unable to benefit from this treatment. Moreover, current clinical adoptive TIL therapy typically requires at least 50 billion cells to achieve effectiveness, resulting in lengthy ex vivo expansion times before TILs can be reinfused into patients, often causing patients to miss the optimal treatment window. Furthermore, following TIL reinfusion, patients often require high-dose IL-2 administration to maintain TIL expansion and activation in vivo. However, these high IL-2 concentrations can damage kidney and liver function. Therefore, there remains a need to improve TIL therapy in this field.

Oncolytic viruses are another promising alternative therapy for refractory cancers. Theoretically, virus-mediated oncolysis can spread to all cancer cells throughout the tumor mass, and the selective infection and lysis of tumor cells by viruses can cooperatively disrupt immunosuppression in the tumor microenvironment and reactivate anti-tumor immunity. However, clinical experience has demonstrated that the anti-viral immune responses triggered by oncolytic virus administration can limit the efficacy of the oncolytic virus monotherapy. Moreover, stromal cells within the tumor microenvironment can hinder viral delivery to cancer cells, thereby restricting virus-induced anti-tumor responses. Rapid apoptosis of initially infected tumor cells can also affect the kinetics of intratumoral viral replication. Consequently, although some oncolytic viruses have progressed to clinical trials, their therapeutic efficacy remains to be improved.

Various combination therapies have been proposed to enhance therapeutic efficacy in cancer treatment. For example, Sonia Guedan et al. (CAR-T Cells and Oncolytic Viruses: Joining Forces to Overcome the Solid Tumor Challenge, Front. Immunol. 9:2460, doi: 10.3389/fimmu.2018.02460) proposed several possible mechanisms by which oncolytic viruses can overcome the multiple obstacles faced by adoptive T cell therapy in solid tumors. These mechanisms include: (1) reversing the immunosuppressive tumor microenvironment through danger signals triggered by oncolytic virus infection, thereby enhancing CAR-T cell trafficking, proliferation, and persistence within the tumor microenvironment; (2) promoting anti-tumor adaptive immune responses through direct tumor cell lysis by oncolytic viruses and the release of tumor-associated antigens; and (3) enhancing T cell effector functions using armed oncolytic viruses engineered with transgenes.

WO2020/056228 describes a combination cancer therapy involving oncolytic viruses and CAR-T cells, wherein the oncolytic viruses express type I interferon and the CAR-T cells are engineered to express interferon a/B receptor transgenes, thereby modulating the function of the CAR-T cells and enhancing their expansion.

WO2018081789 discloses a method of enhancing the expansion of tumor-infiltrating lymphocytes (TILs) using engineered antigen-presenting cells (aAPCs) and treating cancer using the expanded TILs. In the method, to construct the aAPCs, myeloma cells that endogenously express HLA-A/B/C, ICOS-L, and CD58 molecules are selected and transduced with viruses to express exogenous CD86 and 4-1BBL and/or OX40L molecules.

Victor Cervera-Carrascon et al. (Comparison of Clinically Relevant Oncolytic Virus Platforms for Enhancing T Cell Therapy of Solid Tumors, Molecular Therapy: Oncolytics Vol. 17 Jun. 2020, https://doi.org/10.1016/j.omto.2020.03.003.) compared four different oncolytic viruses (adenovirus, vaccinia virus, herpes simplex virus and reovirus) for their impact on TIL adoptive therapy in solid tumors. The study assessed tumor growth inhibition and survival rates in tumor-bearing animal models treated with TIL therapy. The results showed that adenovirus, when combined with TIL therapy, was the only virus to significantly reduce tumor volume compared to TIL therapy alone (TIL+PBS). None of the other viruses demonstrated significant tumor growth inhibition relative to the PBS control. Accordingly, in terms of complete response rates, TIL+PBS achieved a response rate of 17.5%, while TIL combined with adenovirus reached 62.5%. In contrast, the response rates for TIL combined with other viruses were all lower than the PBS control, specifically: herpes simplex virus (0%), vaccinia virus (12.5%), and reovirus (12.5%). However, the underlying reasons for the significant differences observed among these oncolytic viruses in adoptive TIL therapy remain unclear.

Given the complexity of cancer treatment and the limitations of current cancer treatment regimens, there remains a need in the field to develop new approaches for cancer therapy.

### SUMMARY OF THE INVENTION

The inventors have surprisingly discovered and disclosed, for the first time, a method for improving immune cell-based anti-tumor immunotherapies, particularly those involving tumor-infiltrating lymphocytes (TILs), by using armed recombinant oncolytic viruses to substantially enhance the antigen-presenting capability of tumor cells within tumor tissue. More specifically, the inventors have found that administering one or more armed recombinant oncolytic viruses expressing a combination of trimeric OX40L and IL-12, or trimeric OX40L, IL-12, and a PD-1 inhibitor, can effectively convert tumor cells in cancer patients into antigen-presenting cells (APCs), which express high levels of MHC-I, MHC-II, and co-stimulatory molecules such as CD80/CD86. As a result, the infiltration, expansion, and activation of anti-tumor-specific lymphocytes (e.g., TILs) in tumor tissue can be restored and/or enhanced, thereby improving therapeutic outcomes. Based on this finding, the inventors have developed recombinant oncolytic virus compositions that upon infecting tumor cells, deliver two factors (trimeric OX40L and IL-12), and recombinant oncolytic virus compositions that upon infecting tumor cells, deliver three factors (trimeric OX40L, IL-12, and a PD-1 inhibitor), as well as their use in cancer treatment and for improving adoptive TIL cell therapy. In further studies, the inventors have also found that, as an alternative to embodiments where the recombinant oncolytic virus composition is used to provide a PD-1 inhibitor, the subject can be treated with a two-factor recombinant oncolytic virus, as described in the invention, in combination with a PD-1 inhibitor. As demonstrated in the Examples, when the two-factor combination (trimeric OX40L and IL-12) or the three-factor combination (trimeric OX40L, IL-12, and PD-1 inhibitor) is provided to the subject using the described compositions and methods, a synergistic effect is achieved in inducing APC conversion of tumor cells, and in enhancing TIL-based anti-tumor immunity.

Therefore, in one aspect, the invention provides at least one recombinant oncolytic virus (herein also referred to as an armed oncolytic virus) comprising a nucleic acid encoding both trimeric OX40L and IL-12, particularly herpes simplex virus, for use in cancer treatment to convert tumor cells into APCs and/or to enhance antigen presentation of tumor cells. Within the context of the uses, the at least one recombinant oncolytic virus can be further combined with a PD-1 inhibitor. The PD-1 inhibitor used in the combination may be a separate PD-1 inhibitor, a composition comprising a PD-1 inhibitor, or it may be produced by the at least one oncolytic virus through incorporating and expressing a nucleic acid encoding the PD-1 inhibitor in its genome. Within the context of the uses, the at least one recombinant oncolytic virus (or its combination with a PD-1 inhibitor) can be further combined with an adoptive cell therapy composition, particularly an adoptive TIL cell therapy composition. Without being bound by any theory, tumor cells converted into antigen-presenting cells (APCs) are believed to promote the recruitment and infiltration of tumor-infiltrating lymphocytes (TILs)-including, but not limited to, pre-existing lymphocytes present in patients before treatment, those induced by treatment, and/or adoptively transferred TILs-into the tumor tissue, and/or their expansion and/or activation.

In another aspect, the invention provides a method for converting tumor cells into antigen-presenting cells (APCs) in a subject, a method for treating cancer, and a method for improving adoptive cell therapy in a cancer patient, wherein the methods comprise administering to the subject at least one recombinant oncolytic virus comprising a nucleic acid encoding trimeric OX40L and IL-12. In a preferred embodiment, the methods further comprise administering a PD-1 inhibitor or a recombinant oncolytic virus comprising a nucleic acid encoding a PD-1 inhibitor to the subject. In another preferred embodiment, the methods further comprise administering an adoptive cell therapy composition, particularly an adoptive TIL cell therapy composition, to the subject.

Therefore, in some embodiments, the invention provides a method for converting tumor cells into antigen-presenting cells (APCs) in a subject, a method for treating a cancer patient, or a method for improving adoptive cell therapy in a cancer patient, wherein the method comprises administering to a subject in need thereof:
a) a recombinant oncolytic virus composition, or
b) a recombinant oncolytic virus composition and a PD-1 inhibitor, or
c) (a) or (b) and an adoptive cell therapy composition,
wherein the recombinant oncolytic virus composition comprises at least one (e.g., one, two or three, preferably two) recombinant oncolytic virus, wherein the at least one recombinant oncolytic virus, upon infecting the subject's tumor cells, expresses exogenous trimeric OX40L and IL-12 and optionally a PD-1 inhibitor,
wherein the adoptive cell therapy composition comprises tumor-infiltrating lymphocytes (TILs), preferably wherein the TILs are derived from the same subject from whom the tumor cells originate. Preferably, the recombinant oncolytic virus composition is a recombinant oncolytic virus composition according to the present invention that provides the two-factor combination of trimeric OX40L and IL-12.

In some other embodiments, the invention provides a method for improving adoptive cell therapy in a subject, comprising administering to the subject in need thereof
a) a recombinant oncolytic virus composition, or
b) a recombinant oncolytic virus composition and a PD-1 inhibitor,
wherein the recombinant oncolytic virus composition comprises at least one (e.g., one, two or three, preferably two) recombinant oncolytic virus, wherein the at least one recombinant oncolytic virus, upon infecting the subject's tumor cells, expresses exogenous trimeric OX40L and IL-12 and optionally a PD-1 inhibitor,
wherein the adoptive TIL therapy comprises administering to the subject an adoptive cell therapy composition comprising tumor-infiltrating lymphocytes (TILs), wherein preferably the TILs are derived from the same subject from whom the tumor cells originate. Preferably, the recombinant oncolytic virus composition is a recombinant oncolytic virus composition according to the present invention that provides the two-factor combination of trimeric OX40L and IL-12.

In some embodiments of the methods of the present invention described above, the methods comprise administering to the subject the combination of:
(a) one or more (preferably one or two) armed oncolytic viruses comprising a nucleic acid encoding both trimeric OX40L and IL-12, or a nucleic acid encoding trimeric OX40L, IL-12 and a PD-1 inhibitor, as exogenous arming genes, and
(b) adoptive TIL cells.

The combination administration of the armed viruses and adoptive TIL cells enhances anti-tumor effects. The combination administration may be concomitant, separate, or sequential administration of the armed viruses and adoptive TIL cells in any order. Compared to administering either the armed viruses or adoptive T cells alone, the combination administration results in a synergistic effect.

In some other embodiments of the methods of the present invention described above, the methods comprise administering to the subject the combination of:
(a) one or two armed oncolytic viruses comprising a nucleic acid encoding trimeric OX40L and IL-12 (but preferably not encoding a PD-1 inhibitor) as exogenous arming genes,
(b) a PD-1 inhibitor, and
(c) adoptive TIL cells.

The combination administration of the armed virus, the PD-1 inhibitor and adoptive TIL cells enhances anti-tumor effects. Compared to administering either the armed viruses or the PD-1 inhibitor alone or administering adoptive T cells alone, the combination administration results in a synergistic effect. The combination administration may be concomitant, separate, or sequential administration of the armed viruses, the PD-1 inhibitor and adoptive TIL cells in any order.

In any embodiments of the methods of the present invention described above, the cancer may be a solid tumor, e.g., head and neck cancer or oral cancer, such as gingival cancer, buccal cancer, and tongue cancer, or a gastrointestinal cancer such as colorectal cancer, pancreatic cancer, or glioblastoma or melanoma, and metastases thereof; preferably the cancer is a squamous cell carcinoma or adenocarcinoma. In some embodiments, the cancer exhibits low tumor infiltration.

In some further aspects, the invention also provides a recombinant oncolytic virus composition, the composition comprising at least one recombinant oncolytic virus, e.g., one, two or three, preferably two recombinant HSV-1 oncolytic viruses, wherein the at least one recombinant oncolytic virus expresses at least 2 (e.g., 1-4) exogenous arming genes upon infection of cells (preferably tumor cells), and wherein the exogenous arming genes comprise trimeric OX40L and IL-12 and optionally a PD-1 inhibitor. Preferably, the composition comprises a first oncolytic virus encoding trimeric OX40L and a PD-1 inhibitor and a second oncolytic virus encoding IL-12 and a PD-1 inhibitor, or the composition comprises a single recombinant oncolytic virus encoding both trimeric OX40L and IL-12.

In some further aspects, the present disclosure provides a combination of the recombinant oncolytic virus composition according to the present invention with:
(a) a PD-1 inhibitor,
(b) an adoptive cell therapy composition for adoptive cell therapy, or
(c) a combination of (a) and (b).

In some further aspects, the present disclosure provides a medicament, kit or combination product comprising said combination, preferably wherein said adoptive cell therapy composition, said PD-1 inhibitor, and said at least one recombinant oncolytic virus are prepared in separate formulations. Preferably, the different recombinant oncolytic viruses included in the at least one recombinant oncolytic virus are prepared in one or, preferably, multiple separate formulations, such as in a second formulation, or in a second and a third formulation.

The present disclosure also provides the use of the recombinant oncolytic virus composition or the combination according to the present invention, in the preparation of a medicament or kit or combination product for use in any one of the methods and/or uses of the present invention described above.

In any of the aspects and embodiments described above, the at least one recombinant oncolytic virus comprised in the recombinant oncolytic virus composition according to the present invention has one or more of the following preferred features.

In some preferred embodiments, the at least one (e.g., one or two) recombinant oncolytic virus comprises heterologous polynucleotides encoding trimeric OX40L and IL-12 in its genome. In some preferred aspects, the at least one recombinant oncolytic virus further comprises a heterologous polynucleotide encoding a PD-1 inhibitor in its genome. In other preferred aspects, the at least one recombinant oncolytic virus does not comprise a heterologous polynucleotide encoding a PD-1 inhibitor in its genome. In yet other preferred aspects, the at least one recombinant oncolytic virus comprises, as its exogenous arming genes, only the heterologous polynucleotides encoding trimeric OX40L and IL-12. In some further preferred aspects, the at least one recombinant oncolytic virus comprises only the heterologous polynucleotides encoding trimeric OX40L, IL-12 and a PD-1 inhibitor as its exogenous arming genes.

Preferably, the at least one recombinant oncolytic virus is one recombinant oncolytic virus comprising a trimeric OX40L-encoding nucleic acid and an IL-12-encoding nucleic acid in its genome. Preferably, the trimeric OX40L-encoding nucleic acid and the IL-12-encoding nucleic acid are located at different genomic loci of the virus.

More preferably, the at least one recombinant oncolytic virus consists of a first and a second recombinant oncolytic virus, wherein the first recombinant oncolytic virus comprises in its genome a nucleic acid encoding trimeric OX40L; the second recombinant oncolytic virus comprises in its genome a nucleic acid encoding IL-12. Even more preferably, the at least one recombinant oncolytic virus also provides a PD-1 inhibitor, which may be e.g. present in the first recombinant oncolytic virus, the second recombinant oncolytic virus, or in both. Thus, in some more preferred embodiments, the first recombinant oncolytic virus and/or the second recombinant oncolytic virus (preferably both) further comprise(s) in its genome a nucleic acid encoding a PD-1 inhibitor, preferably wherein the nucleic acid encoding the PD-1 inhibitor is located at a different genomic locus of the virus from the nucleic acid encoding OX40L or the nucleic acid encoding IL-12, respectively.

In any one of the embodiments described above, preferably the recombinant oncolytic virus(es) is a herpes simplex virus type 1 (HSV-1). More preferably, in any one of the embodiments described above, the nucleic acids encoding the OX40L, IL-12, and optionally the PD-1 inhibitor, which are included in the genome of the recombinant oncolytic virus(es), are inserted into the following HSV-1 virus loci:
When the nucleic acids encoding OX40L and IL-12 are provided in different oncolytic viruses (preferably in a first and a second oncolytic virus, respectively), the nucleic acid encoding OX40L is inserted into the HSV-1 virus ICP34.5 locus, preferably with two copies inserted individually into the two virus ICP34.5 loci;
   the nucleic acid encoding IL-12 is inserted into the HSV-1 virus ICP34.5 locus, preferably with two copies inserted individually into the two virus ICP34.5 loci;
   and optionally, the nucleic acid encoding the PD-1 inhibitor is inserted into the HSV-1 virus intergenic region between UL26 and UL27;
When the nucleic acids encoding OX40L and IL-12 are provided in the same oncolytic virus, they are inserted individually into different loci of the HSV-1 genome, for example:
   the nucleic acid encoding OX40L is inserted into the HSV-1 virus ICP34.5 locus, preferably with two copies inserted individually into the two virus ICP34.5 loci;
   the nucleic acid encoding IL-12 is inserted into a different HSV-1 virus genomic locus, such as the intergenic region between UL26 and UL27 or the intergenic region between UL3 and UL4, with a preference for the intergenic region between UL26 and UL27,
wherein the oncolytic virus may comprise, but more preferably does not comprise, a nucleic acid encoding a PD-1 inhibitor.

In any one of the embodiments described above, preferably, each of the at least one recombinant oncolytic virus comprises 1-4 (e.g., 1, 2, 3 or 4), preferably no more than 3 (e.g., 1, 2 or 3), more preferably no more than 2 (e.g., 1 or 2) exogenous arming genes. In further preferred embodiments, the at least one recombinant oncolytic virus comprises a total of 2-10 (e.g., 2, 3, 4, 5, 6, 7, 8, 9, or 10) exogenous arming genes, e.g., 2 to 6 exogenous arming genes, preferably 2 to 4, e.g., 3 or 2, exogenous arming genes.

Thus, in some most preferred embodiments, the present disclosure provides a two-factor recombinant oncolytic virus, wherein the recombinant oncolytic virus comprises two exogenous arming genes, selected from the group consisting of:
(a) a polynucleotide encoding trimeric OX40L and a polynucleotide encoding a PD-1 inhibitor (preferably an anti-PD-1 single-chain antibody);
(b) a polynucleotide encoding IL-12 and a polynucleotide encoding a PD-1 inhibitor (preferably an anti-PD-1 single-chain antibody); and
(c) a polynucleotide encoding trimeric OX40L and a polynucleotide encoding IL-12.

Preferably, the recombinant oncolytic virus composition of the present invention comprises one or more two-factor recombinant oncolytic viruses according to the present invention. In some embodiments, the recombinant oncolytic virus composition of the present invention comprises or consists of the two-factor recombinant oncolytic virus defined in (c) above, and in some preferred instances, the recombinant oncolytic virus composition is combined with a PD-1 inhibitor (e.g., a composition comprising a PD-1 inhibitor). In some other embodiments, the recombinant oncolytic virus composition of the present invention comprises or consists of the two-factor recombinant oncolytic viruses defined in (a) and (b) above.

Any nucleic acid capable of encoding an exogenous trimeric OX40L can be used in the present invention. Preferably, the nucleic acid encodes a trimeric OX40L polypeptide that comprises from N-terminus to C-terminus a trimerization domain (e.g., a trimerization domain from a human TRAF family member such as TRAF2, e.g., amino acids 310-349 of human TRAF2), an extracellular domain of OX40L (e.g., amino acids 51-183 of human OX40L) and a transmembrane domain (e.g., a PDGFR transmembrane domain). Preferably, the polypeptide comprises the amino acid sequence of SEQ ID NO: 18 or an amino acid sequence having at least 90%, 91%, 92%, 93%, 94%, 95%, 96%, 97%, 98%, or 99% identity thereto.

Any nucleic acid capable of encoding IL-12 can be used in the present invention. Preferably, the nucleic acid encodes an IL-12 heterodimeric protein comprising or consisting of an IL-12a polypeptide and an IL-12β polypeptide. Preferably, the IL-12a polypeptide comprises the amino acid sequence of SEQ ID NO: 17 or an amino acid sequence having at least 90%, 95%, 96%, 97%, 98%, or 99% identity thereto; and the IL-12β polypeptide comprises the amino acid sequence of SEQ ID NO: 16 or an amino acid sequence having at least 90%, 95%, 96%, 97%, 98%, or 99% identity thereto.

Any nucleic acid capable of encoding a PD-1 inhibitor can be used in the present invention. Preferably, the nucleic acid encodes an anti-PD-1 antibody, preferably an anti-PD-1 single-chain scFv antibody, more preferably wherein the anti-PD-1 scFv antibody comprises the VH amino acid sequence of SEQ ID NO: 20 and the VL amino acid sequence of SEQ ID NO: 21. Preferably, the nucleic acids encoding the trimeric OX40L, IL-12 and PD-1 inhibitor are operably linked to a CMV promoter.

In a preferred embodiment of the present invention, the recombinant oncolytic virus composition according to the present invention provides all three therapeutic factors of the invention, IL-12, OX40L and a PD-1 inhibitor, and preferably comprises or consists of a first two-factor recombinant oncolytic virus encoding IL-12 and a PD-1 inhibitor and a second two-factor recombinant oncolytic virus encoding trimeric OX40L and a PD-1 inhibitor. In another, even more preferred embodiment, the recombinant oncolytic virus composition of the present invention provides the IL-12 and OX40L factors of the three therapeutic factors, and depending on considerations such as the tumor cell type or the specific condition of the patient to be treated, in some embodiments, is preferably combined with a composition comprising a PD-1 inhibitor. In such instances, preferably, the recombinant oncolytic virus composition of the present invention comprises or consists of a single-factor recombinant oncolytic virus encoding trimeric OX40L and a second recombinant oncolytic virus encoding IL-12, or comprises or consists of a single recombinant oncolytic virus encoding both trimeric OX40L and IL-12. Preferably, in these described embodiments, the trimeric OX40L polypeptide according to the present invention has the amino acid sequence of SEQ ID NO: 18; the IL-12 according to the present invention comprises an IL-12a having the amino acid sequence of SEQ ID NO: 17 and an IL-12β having the amino acid sequence of SEQ ID NO: 16; and the PD-1 inhibitor according to the present invention is an anti-PD-1 single-chain scFv antibody comprising the HCDR1-HCDR3 amino acid sequences of SEQ ID NOs: 22-24 and the LCDR1-LCDR3 amino acid sequences of SEQ ID NOs: 25-27, preferably comprising the VH amino acid sequence of SEQ ID NO: 20 and the VL amino acid sequence of SEQ ID NO: 21, more preferably wherein the scFv antibody comprises or consists of the amino acid sequence of SEQ ID NO: 19.

### BRIEF DESCRIPTION OF THE DRAWINGS

FIGS. 1A-1B show that activated OC1-TILs have the ability to specifically destroy tumor cells, as measured by ELISA in tumor cell cytotoxicity assays, where *** indicates p<0.001 relative to the TIL group.

FIG. 2 shows schematic diagrams illustrating the engineering of an oncolytic virus encoding OX40L (OV-OX40L), an oncolytic virus encoding IL-12 (OV-IL-12), and a two-factor oncolytic virus encoding both OX40L and IL-12 (OV-OX40L/IL-12).

FIGS. 3A-3E show characterization of the OV-OX40L, OV-IL-12 and OV-OX40L/IL-12 oncolytic viruses using PCR, Western blot, flow cytometry and ELISA.

FIGS. 4A-4D show the cytotoxic effects of oncolytic viruses on primary oral cancer cells and tissues. FIG. 4A compares the cytotoxic effects of different oncolytic viruses (including the GFP-expressing OV-GFP virus, the trimeric OX40L-expressing OV-OX40L virus, the IL-12-expressing OV-IL-12 virus and the OV-OX40L/IL-12 virus expressing both trimeric OX40L and IL-12), at various virus titers on primary tumor cells from multiple oral cancer patients (OC1, OC2, OC3 and OC4). FIGS. 4B-4D show the cytotoxic effects of the oncolytic viruses on samples from OC1 primary oral cancer tissue, where ** indicates p<0.01, *** indicates p <0.001, relative to tissue block-1; ## indicates p<0.01, ### indicates p<0.001, relative to tissue block-3. FIG. 4E shows that the oncolytic viruses can infect various tumor cell lines, including glioblastoma, fibrosarcoma, colon cancer, and breast cancer.

FIG. 5 shows the cytotoxic effects of the oncolytic viruses, OV-OX40L/IL-12, OV-OX40L/αPD-1, OV-IL-12/αPD-1, and OV-OX40L/IL-12/αPD-1, in combination with TILs on primary oral cancer cells, as assessed by co-culture assays.

FIGS. 6A-6C show the activation of TILs by primary oral cancer cells pre-infected with OV-OX40L/IL-12, OV-OX40L/αPD-1, OV-IL-12/αPD-1 and OV-OX40L/IL-12/αPD-1, as measured by ELISA and ELISPOT. In FIG. 6A, *** indicates p<0.001 relative to OC+OV-GFP+TIL. In FIG. 6B, *** indicates p<0.001 relative to OC1+OV-GFP+TIL.

FIG. 7 shows the cytotoxic effects of the oncolytic viruses OV-OX40L/IL-12, OV-OX40L/αPD-1, OV-IL-12/αPD-1, and OV-OX40L/IL-12/αPD-1 in combination with TILs on primary oral cancer cells, as determined by MTT assays, where *** indicates p<0.001 relative to OC+OV-GFP+TIL.

FIG. 8 shows T cell expansion stimulated by primary oral cancer cells pre-infected with OV-OX40L/IL-12, OV-OX40L/αPD-1, OV-IL-12/αPD-1 and OV-OX40L/IL-12/αPD-1, as measured by MTT assays, where *** indicates p<0.001 relative to TIL+OC-GFP.

FIG. 9 shows TIL activation by OC1 primary oral cancer cells pre-infected with OV-OX40L/IL-12, OV-OX40L/αPD-1, OV-IL-12/αPD-1 and OV-OX40L/IL-12/αPD-1, as measured by flow cytometry, where *** indicates p<0.001 relative to TIL+OC1-OV.

FIG. 10 shows the effects of the armed oncolytic viruses OV-OX40L/IL-12, OV-OX40L/αPD-1, OV-IL-12/αPD-1 and OV-OX40L/IL-12/αPD-1 on antigen expression on the surface of primary oral cancer cells, as measured by flow cytometry, where *** indicates p<0.001 relative to OC1+OV+TIL.

FIG. 11 shows the effects of the viruses OV-OX40L/IL-12, OV-OX40L/αPD-1, OV-IL-12/αPD-1 and OV-OX40L/IL-12/αPD-1 on the surface antigen expression of primary oral cancer cells, as measured by qPCR.

FIGS. 12A-12D show the inhibition of OC1- and OC4-PDX tumor growth by OV-OX40L/IL-12 in combination with TILs, as evaluated in immunodeficient mice. FIGS. 12A and 12B show tumor growth in OC1-PDX tumor-bearing mice under different dosing regimens, where *** indicates p<0.001 relative to OC1+TIL. FIGS. 12C and 12D show tumor growth in OC4-PDX tumor-bearing mice under different dosing regimens, where *** indicates p<0.001 relative to OC4+TIL.

FIG. 13 shows IFN-γ levels in tumor tissues from different groups, as detected by ELISA, where ** indicates p<0.01, *** indicates p<0.001, both relative to OC1+TIL; ## indicates p <0.01 relative to OC1+OV-GFP+TIL.

FIGS. 14A and 14B show tumor growth curves of MC38 graft tumors and survival curves of mice, as assessed in immunocompetent mice treated with different regimens.

FIGS. 15A and 15B show tumor growth curves of Pan02-HVEM graft tumors and survival curves of mice, as assessed in immunocompetent mice treated with different regimens.

FIGS. 16A-16D show the expression of the markers on the surface of immune cells and tumor cells in tumor tissues at 3 and 7 days after treatment with OV-OX40L/IL-12+αPD-1 in combination with TILs.

### DETAILED DESCRIPTION OF THE INVENTION

Unless otherwise defined, all technical and scientific terms used herein have the same meaning as commonly understood by one of ordinary skill in the art to which this invention belongs. All publications, patent applications, patents and other references mentioned herein are incorporated by reference in their entirety. In addition, the materials, methods, and examples are illustrative only and not intended to be limiting.

### I. Definitions

The term “about” when used in conjunction with a numerical value means encompassing numerical values within a range plus or minus 5% of the specified numerical value. The term is also intended to encompass numerical values within ±1%, ±0.5%, or ±0.1% of the specified numerical value.

As used herein, the terms “comprise” and “include” are intended to encompass the stated elements, integers, or steps, but do not exclude the presence of additional elements, integers, or steps.

As used herein, the terms “first”, “second”, “third” etc., are used to distinguish stated elements and, unless otherwise specified, are not intended to indicate that the elements shall have a specific quantity, or shall be present in any particular order or position.

As used herein, the term “and/or” refers to any single element, or any and every possible combination of more of the listed elements.

The terms “co-administration”, “combination administration”, and “administered in combination with” are intended to mean two or more pharmaceutical active agents are administered to a subject in a manner so that the pharmaceutical active agents and/or their metabolites can be simultaneously present in the subject. For instance, in the methods and/or uses according to the present invention, a first and a second pharmaceutical active agents may be administered in combination to a subject, wherein the first pharmaceutical active agents comprises adoptive TIL cells, and the second pharmaceutical active agents comprises one or more recombinant oncolytic viruses that provide the two-factor combination of the invention (trimerized OX40 and IL-12) or the three-factor combination of the invention (trimeric OX40L, IL-12, and a PD-1 inhibitor). In some instances where the one or more recombinant oncolytic viruses provide only the two factors of the invention (trimerized OX40 and IL-12), a third pharmaceutical active agent—a PD-1 inhibitor or a composition containing a PD-1 inhibitor—may also be administered to the subject as appropriate.

As used herein, “co-administration”/“combination administration” includes simultaneous administration of separate compositions, administration of separate compositions at different times, or administration of a composition comprising two or more pharmaceutical active agents. Preferably, according to the present invention, the adoptive T cells, the recombinant oncolytic virus(es), and optionally the PD-1 inhibitor are administered separately in individual compositions, and preferably, the recombinant oncolytic virus(es) is administered prior to the infusion of the adoptive T cells into the subject, allowing the viruses to infect the subject's tumor cells and express the factors of the invention—trimeric OX40L and IL-12, and optionally the PD-1 inhibitor—carried by the viruses.

As used herein, an “effective amount” or “therapeutically effective amount” refers to an amount of a pharmaceutical active agent that is sufficient to provide a desired biological effect after administration at one or more dosages over a period. The desired biological effect may be alleviation, cure, or reduction of a disease or one or more symptoms of the disease, or an improvement in survival of the subject. In cancer treatment, the desired biological effects may include a reduction in tumor burden, decrease in tumor volume, or eradication of the tumor; inhibition (e.g. slowing or halting) of infiltration of cancer cells into peripheral organs; inhibition of metastatic tumor growth; inhibition (stabilization or halting) of tumor growth; and/or induction and promotion of an antitumor immune response. Suitable effective amounts can be determined by those skilled in the art based on the whole teachings of the present disclosure and using routine experimentation and analysis. Therapeutically effective amounts may vary depending on factors such as the specific active agents used (e.g. recombinant oncolytic viruses, adoptive TIL cells, and optionally PD-1 inhibitor), age and condition of the subject to be treated, stage of tumor formation, presence of other treatment modalities, etc. Similarly, the dosages for administering a composition, including a recombinant oncolytic virus composition, an adoptive cell therapy composition, and a composition containing a PD-1 inhibitor, are determined by factors such as the active agents involved, routes of administration, the subject's age and condition, and the physician's judgment, among others.

The terms “individual”, “subject” or “patient” are used interchangeably herein and refer to a mammal. Mammals include, but are not limited to, domesticated animals (e.g., cattle, sheep, cats, dogs, and horses), primates (e.g., humans and non-human primates such as monkeys), rabbits and rodents (e.g., mice and rats). In particular, the individual is a human.

The term ‘treatment’ refers to clinical intervention intended to alter the natural course of a disease in the individual being treated. Intended therapeutic outcomes include, but are not limited to, preventing occurrence or recurrence of a disease, alleviation of a symptom, diminishment of any direct or indirect pathological consequences of a disease, preventing metastasis, decreasing the rate of disease progression, amelioration or palliation of the disease state, and alleviation or improvement of prognosis. In some embodiments of the present invention, the administration of the recombinant oncolytic virus composition according to the invention, or the administration of the recombinant oncolytic virus composition in combination with the adoptive TIL therapy composition, and optionally the PD-1 inhibitor, delays cancer progression or slows the advancement of cancer.

The term “antitumor activity” or “antitumor effect” refers to a biological effect that can be manifested by one or more of the following, among others: a reduction in tumor volume, a reduction in the number of tumor cells, a reduction in tumor cell proliferation, or an increase in survival of a cancer patient.

The terms “tumor” and “cancer” are used interchangeably herein and encompass solid and liquid tumors.

As used herein, the term “recombinant” when used in reference to, e.g., a virus, cell, nucleic acid, protein, or vector, refers to a virus, cell, nucleic acid, protein, or vector that has been modified by the introduction of a heterologous nucleic acid or protein, or by alteration of its native nucleic acid or protein sequence, or refers to material derived from such a modified virus or cell.

As used herein, a “recombinant HSV-1 oncolytic virus” refers to a herpes simplex virus type 1 (HSV-1) that has been genetically engineered to carry a heterologous polynucleotide, such as a polynucleotide encoding trimeric OX40L, IL-12 and/or a PD-1 inhibitor, wherein the virus is capable of selectively infecting tumor cells and exhibits oncolytic activity. The wild-type HSV-1, being a neurotropic virus, is highly prevalent in the human population with mild clinical manifestations. As a double-stranded DNA virus, the HSV-1 genome is 152 kb long, consisting of a unique long region (UL) and a unique short region (US), with terminal inverted repeat sequences TRL and TRs at both ends, and internal inverted repeat sequences (IR) joining the two regions. The IR includes IRL and IRs, which are inverted repeats of TRL and TRs, respectively. Recombinant oncolytic HSV-1 viruses can be engineered from clinical isolates by deleting one or more HSV-1 genes and optionally inserting genes related to immune activation and tumor therapy. The HSV-1 ICP34.5 (also known as γ34.5) is a neurovirulence gene encoding a protein required for HSV-1 replication in neuronal cells. Clinical studies have demonstrated that a recombinant HSV-1 virus, engineered with a deletion of the γ34.5 gene, selectively replicates within and lyses tumor cells. Furthermore, the knock-out of the ICP47 gene, in combination with the γ34.5 deletion, results in a recombinant virus that enhances MHC-I expression and antigen presentation in infected cells. Therefore, in certain preferred embodiments, the recombinant oncolytic virus of the present invention comprises a single-or double-copy knockout of the ICP34.5 gene, together with a knockout of the ICP47 gene in its genome. More preferably, the recombinant oncolytic virus of the present invention is an HSV-1 virus comprising a double-copy deletion of ICP34.5 and a deletion of ICP47, i.e., an HSV-1 virus in which both copies of the ICP34.5 gene and the ICP47 gene are knocked out.

The terms “knockout”, “gene knockout”, and “gene deletion” are used herein to refer to a gene that has been disrupted through genetic engineering, thereby losing its functions. For example, genetic engineering can introduce a null mutation or insert a heterologous nucleic acid into the gene, preventing it from being expressed or allowing only minimal expression insufficient for its original biological activity, or resulting in a non-functional gene product.

The term “host cell” refers to a cell into which an exogenous polynucleotide has been introduced, including the progeny of such a cell. Host cells include in vitro cultured cells as well as cells within a transgenic animal individual or tissue. For example, in some instances, the host cells may be a tumor cell, e.g., a tumor cell isolated from a subject; or a tumor cell within a subject, into which an exogenous encoding polynucleotide has been introduced via a recombinant virus.

The terms “exogenous” or “heterologous”, used interchangeably when describing a nucleic acid or a protein, mean that the nucleic acid or protein is foreign to the host cell intended to contain or already containing the nucleic acid or protein, i.e., the location of the nucleic acid or protein within the host cell is not its native location in nature. A heterologous nucleic acid sequence also refers to a sequence introduced (e.g., through viral infection) into the same host cell or subject from which it is derived, but it is present in a non-naturally occurring state, e.g., present at a different location, in a different copy number, or under the control of a different regulatory element.

The term “regulatory sequence” or “expression control sequence” refers to a nucleic acid sequence that induces, suppresses, or otherwise controls the transcription of a coding nucleic acid sequence operably linked thereto. Regulatory sequences include, but are not limited to, initiation sequences, enhancer sequences, intron sequences, and promoter sequences.

The term “expression cassette” refers to a DNA sequence encoding and capable of expressing one or more genes of interest (e.g., the factors of the invention, trimeric OX40L, IL-12, and PD-1 inhibitor). In an expression cassette, typically, a heterologous polynucleotide sequence encoding the gene(s) of interest is operably linked to an expression control sequence. Depending on the insertion site and intended function, the insertion of an expression cassette may, in some instances, disrupt the gene located at the insertion site, while in some other instances, have no effect on the transcription and/or expression of the genes flanking the insertion site.

The term “operably linked” or “operably connected” refers to an arrangement in which the stated components are placed in a relationship so that they can function in the intended manner.

The term “sequence identity” is used to describe the degree of similarity between two amino acid sequences or two polynucleotide sequences. To determine the percent identity between two amino acid sequences or two nucleic acid sequences, the sequences can be aligned for optimal comparison (e.g., gaps can be introduced in one or both of the first and second amino acid or nucleic acid sequences for optimal alignment, or an alignment window suitable for comparison may be chosen). In a preferred embodiment, for comparison purposes, the aligned length of the reference sequence is at least 30% of the full length of the reference sequence, more preferably at least 40%, even more preferably at least 50%, 60%, or even more preferably at least 70%, 80%, 90%, or most preferably 100%. After the alignment, the amino acid residues or nucleotides at corresponding positions in the two sequences are compared. When a position in the first sequence is occupied by the same amino acid residue or nucleotide as the corresponding position in the second sequence, the molecules are identical at that position.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

As used herein, a “conservative” amino acid or nucleotide alteration refers to a neutral or nearly neutral alteration in amino acid or nucleotide that allows the protein or nucleic acid molecule containing such alteration to substantially retain its original function. For example, a conservative amino acid substitution refers to replacing or substituting an amino acid with a different amino acid having a side chain of similar biochemical property (e.g., charge, hydrophobicity, and size). Such conservatively modified variants can be in addition to polymorphic variants, interspecies homologs, or allelic variants. The following 8 groups include amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M). One skilled in the art can readily determine the conservative nature of an amino acid or nucleotide change in a given polypeptide sequence or nucleic acid sequence by routine techniques, such as functional assays.

As used herein, the terms “nucleic acid” and “polynucleotide” can be used interchangeably. For example, a polynucleotide encoding trimeric OX40L can be referred to as a trimeric OX40L encoding nucleic acid. Similarly, a polynucleotide encoding IL-12 can be referred to as an IL-12 encoding nucleic acid; and a polynucleotide encoding a PD-1 inhibitor can be referred to as a PD-1 inhibitor encoding nucleic acid.

The term “OX40L” or “OX40 ligand”, also known as TNFSF4, as used herein, refers to an OX40 ligand that is capable of interacting with the tumor necrosis factor receptor OX40 and transmitting survival and activation signals to T cells expressing OX40 on their surface. An example of an OX40L polypeptide is the human OX40L protein under UniProt accession number P23510. The present invention also encompasses functional fragments and variants of full-length native OX40L proteins, and fusion proteins containing the extracellular domain of OX40L. For example, in a preferred embodiment, the OX40L polypeptide according to the present invention is a membrane-bound fusion protein comprising the extracellular domain of OX40L, wherein the amino acid sequence of the extracellular domain is linked at its N-terminus to a trimerization domain and at its C-terminus to a transmembrane domain. A trimerization domain is a peptide sequence that functions to mediate trimerization of the polypeptide containing it, and such peptide sequences are known in the art. An OX40L polypeptide containing a trimerization domain, referred to herein as “trimeric OX40L”, is a preferred embodiment of the present invention. Preferably, the trimerization domain fused to the OX40L extracellular domain is the trimerization domain of a TRAF family protein, such as amino acids 310-349 of human TRAF2 (e.g., the amino acid sequence under UniProt Q12933). In another preferred embodiment, the OX40L extracellular domain comprised in the fusion protein has the amino acid sequence of Gln51 to Leu183 of OX40L under UniProt P23510, or a variant thereof having an amino acid sequence at least 90%, 91%, 92%, 93%, 94%, 95%, 96%, 97%, 98%, 99% sequence identical thereto, in particular a conservative amino acid substitution variant. In yet another preferred embodiment, the transmembrane domain fused to the OX40L extracellular domain may be derived from a mammalian transmembrane protein, e.g., the transmembrane domain of PDGFR.

In a preferred embodiment, the oncolytic virus of the present invention comprises a nucleic acid encoding a trimeric OX40L, wherein the nucleic acid encodes and expresses a fusion polypeptide comprising, from N- to C-terminus, a TRAF2 trimerization domain (e.g., amino acids 310-349 of human TRAF2), the extracellular domain of OX40L (e.g., amino acids 51-183 of human OX40L), and a transmembrane domain (e.g., PDGFR transmembrane domain). Preferably, upon expression, the fusion polypeptide forms a trimeric OX40L displayed on the cell surface, which is capable of binding to an OX40 molecule via the OX40L extracellular domain and activating associated signaling events.

In one embodiment, the OX40L polypeptide comprises or consists of the amino acid sequence of SEQ ID NO: 18, or comprises or consists of an amino acid sequence having at least 90%, 91%, 92%, 93%, 94%, 95%, 96%, 97%, 98%, 99% sequence identity thereto. In a preferred embodiment, the OX40L polypeptide is a conservative amino acid substitution variant of SEQ ID NO: 18, preferably with no more than 10 amino acid residue changes, e.g., 0-5 changes.

In the present disclosure, an OX40L trimeric encoding gene, encoding nucleic acid, or polynucleotide refers to a nucleic acid capable of encoding a trimeric OX40L polypeptide. When delivered to a tumor cell (e.g., via an oncolytic virus), it expresses a functional trimeric OX40L protein on the surface of the tumor cell. In one embodiment, the OX40L encoding nucleic acid encodes the trimeric OX40L polypeptide of the invention. In one embodiment, the OX40L encoding nucleic acid encodes a polypeptide comprising the amino acid sequence of SEQ ID NO: 18, or an amino acid sequence variant having at least 90%, 91%, 92%, 93%, 94%, 95%, 96%, 97%, 98%, 99% sequence identity thereto, in particular a conservative amino acid substitution variant. In some other embodiments, the OX40L encoding nucleic acid comprises the nucleotide sequence of SEQ ID NO: 3.

The term “IL-12” refers to a heterodimeric protein composed of an IL-12α (p35) subunit and an IL-12β (p40) subunit. An example of an IL-12α subunit is the human IL-12a protein under UniProt accession number P29459. An example of an IL-12β subunit is the human IL-12β protein under UniProt accession number P29460. The present invention encompasses full-length native IL-12α and IL-12β, functional fragments thereof, variants thereof, or fusion proteins containing them.

In one embodiment, the IL-12α polypeptide comprises or consists of the amino acid sequence of SEQ ID NO: 17, or comprises or consists of an amino acid sequence having at least 90%, 91%, 92%, 93%, 94%, 95%, 96%, 97%, 98%, 99% sequence identity thereto. In a preferred embodiment, the IL-12α polypeptide is a conservative amino acid substitution variant of SEQ ID NO: 17, preferably with no more than 10 amino acid residue changes, e.g., 0-5 changes. In another embodiment, the IL-12α polypeptide comprises or consists of the amino acid sequence under UniProt P29459, or comprises or consists of an amino acid sequence having at least 90%, 91%, 92%, 93%, 94%, 95%, 96%, 97%, 98%, 99% sequence identity thereto. In a preferred embodiment, the IL-12α polypeptide is a conservative amino acid substitution variant of the amino acid sequence under UniProt P29459, preferably with no more than 10 amino acid residue changes, e.g., 0-5 changes.

In one embodiment, the IL-12β polypeptide comprises or consists of the amino acid sequence of SEQ ID NO: 16, or comprises or consists of an amino acid sequence having at least 90%, 91%, 92%, 93%, 94%, 95%, 96%, 97%, 98%, 99% sequence identity thereto. In a preferred embodiment, the IL-12β polypeptide is a conservative amino acid substitution variant of SEQ ID NO: 16, preferably with no more than 10 amino acid residue changes, e.g., 0-5 changes. In another embodiment, the IL-12β polypeptide comprises or consists of the amino acid sequence under UniProt P29460, or comprises or consists of an amino acid sequence having at least 90%, 91%, 92%, 93%, 94%, 95%, 96%, 97%, 98%, 99% sequence identity thereto. In a preferred embodiment, the IL-12β polypeptide is a conservative amino acid substitution variant of the amino acid sequence under UniProt P29460, preferably with no more than 10 amino acid residue changes, e.g., 0-5 changes.

In a preferred embodiment, the IL-12 heterodimeric protein comprises or consists of an IL-12α polypeptide and an IL-12β polypeptide, wherein the IL-12α polypeptide comprises or consists of the amino acid sequence of SEQ ID NO: 17, and the IL-12β polypeptide comprises or consists of the amino acid sequence of SEQ ID NO: 16.

In the present disclosure, an IL-12 encoding gene, encoding nucleic acid, or polynucleotide refers to a nucleic acid capable of encoding a functional IL-12. When delivered to a tumor cell (e.g., via an oncolytic virus), it expresses a functional IL-2, which is secreted from the tumor cell or displayed on the surface of the tumor cell. In one embodiment, the IL-12 encoding nucleic acid encodes and expresses secreted forms of IL-12α and IL-12β. In another embodiment, the IL-12 encoding nucleic acid encodes and expresses IL-12 or IL-12β displayed on the cell surface by fusion to a transmembrane domain such as the PDGFR transmembrane domain. In one embodiment, IL-12α and IL-12β are expressed as a polycistronic construct. In another embodiment, the IL-12α encoding nucleic acid and the IL-12β encoding nucleic acid are linked by an IRES sequence, which functions to recruit ribosomes for the translation of mRNA.

In one embodiment, the IL-12 encoding nucleic acid encodes the human IL-12α polypeptide under UniProt P29459 and the human IL-12β polypeptide under UniProt P29460, or variants thereof having amino acid sequences with at least 90%, 91%, 92%, 93%, 94%, 95%, 96%, 97%, 98%, 99% sequence identity thereto, in particular conservative amino acid substitution variants. In another embodiment, the IL-12 encoding nucleic acid encodes an IL-12α polypeptide comprising the amino acid sequence of SEQ ID NO: 17, or a variant thereof having an amino acid sequence with at least 90%, 91%, 92%, 93%, 94%, 95%, 96%, 97%, 98%, 99% sequence identity thereto, in particular a conservative amino acid substitution variant; and an IL-12β polypeptide comprising the amino acid sequence of SEQ ID NO: 16, or a variant thereof having an amino acid sequence with at least 90%, 91%, 92%, 93%, 94%, 95%, 96%, 97%, 98%, 99% sequence identity thereto, in particular a conservative amino acid substitution variant. In some other embodiments, the IL-12 encoding nucleic acid comprises the nucleotide sequences of SEQ ID NO: 1 and SEQ ID NO: 2.

The term “PD-1 inhibitor” refers to a substance that blocks the binding of PD-1 to PD-L1 and the signaling therefrom. An PD-1 inhibitor may be, for example, an inhibitory anti-PD-1 (programmed death 1 protein) antibody or an inhibitory anti-PD-L1 (programmed death ligand 1) antibody. Examples of anti-PD-1 antibodies include, e.g., nivolumab, pembrolizumab, pidilizumab. Examples of anti-PD-L1 antibodies include, e.g., MPDL3280A, MSB00107180. The anti-PD-1 antibody or anti-PD-L1 antibody used in the present invention may be a full-length antibody or an antigen-binding fragment thereof, such as an scFv.

In one embodiment, the PD-1 inhibitor is an anti-PD-1 scFv antibody. In one embodiment, the anti-PD-1 scFv antibody comprises, from N- to C-terminus: a heavy chain variable region (VH) —linker—light chain variable region (VL); or a light chain variable region (VL)—linker—heavy chain variable region (VH). Any linker that can be used to form an scFv antibody while maintaining its ability to bind to the target antigen can be used in the anti-PD-1 scFv antibody of the present invention. In one embodiment, the linker is 10-20 amino acids in length, e.g., a 15 amino acid flexible linker. In a preferred embodiment, the linker is (GnS) m, wherein n and m are integers from 1-5, e.g., n=4, m=2, 3 or 4. Preferably, the linker is (G4S) 3. To facilitate secretory expression of the anti-PD-1 scFv antibody in tumor cells, the antibody may further comprise a signal peptide at the N-terminus, e.g., the amino acid sequence of SEQ ID NO: 26.

As used herein, the term “exogenous arming gene” refers to a nucleic acid or polynucleotide that is inserted into a recombinant oncolytic virus and is capable of expressing and producing a therapeutic molecule (e.g., RNA and protein, preferably protein) when the virus infects a cell, preferably a tumor cell. The nucleic acid or polynucleotide is exogenous to the recombinant oncolytic virus and the host cell (e.g., a tumor cell in vitro or in vivo) into which the virus has been or will be introduced. Such therapeutic exogenous arming genes that can be used to arm a recombinant oncolytic virus include, but are not limited to, any nucleic acids/polynucleotides that can improve the infection/replication ability and/or oncolytic effect of the recombinant oncolytic virus, and/or overcome the immunosuppressive tumor microenvironment (TME), such as nucleic acids/polynucleotides encoding therapeutic proteins (e.g., cytokines), tumor-associated antigens (TAAs), T-cell co-stimulatory molecules, immune checkpoint inhibitors (ICIs), and cell suicide genes. Some specific examples of exogenous arming genes include, but are not limited to, cell death-related molecules that directly induce tumor cell death, such as tumor necrosis factor-related apoptosis-inducing ligand (TRAIL) and the tumor suppressor gene p53; anti-angiogenic molecules such as endostatin and vascular endothelial growth inhibitor (VEGI); immunomodulatory factors such as immune-related cytokines (GM-CSF, IL-2, interferons), chemokines (CCL5, CCL20, CCL21), and other factors that induce anti-tumor immune responses (viral membrane proteins, HSP70); and micro RNA molecules that inhibit tumor-associated genes, such as miRNAs, siRNAs, shRNAs, and lncRNAs. In some embodiments of the present invention, the exogenous arming genes that are introduced into the at least one recombinant oncolytic virus in the recombinant oncolytic virus composition of the present invention may include, but are not limited to (although in some preferred embodiments, are limited to only), the two factors of the invention: an exogenous polynucleotide encoding trimeric OX40L and an exogenous polynucleotide encoding IL-12. In some other embodiments of the present invention, the exogenous arming genes that are introduced into the at least one recombinant oncolytic virus in the recombinant oncolytic virus composition of the present invention may include, but are not limited to (although in some preferred embodiments, are limited to only), the three factors of the invention: an exogenous polynucleotide encoding trimeric OX40L, an exogenous polynucleotide encoding IL-12, and an exogenous polynucleotide encoding a PD-1 inhibitor.

Although multiple exogenous arming genes can be introduced into a single recombinant oncolytic virus, in some instances, this is unfavorable for the recombinant oncolytic virus to maintain the stability. Therefore, within the at least one recombinant oncolytic virus of the recombinant oncolytic virus composition of the present invention, the number of exogenous arming genes inserted into each single recombinant oncolytic virus is preferably no more than four, more preferably no more than three, and most preferably no more than two. For example, in some preferred embodiments, the exogenous nucleic acids encoding trimeric OX40L, IL-12, and PD-1 inhibitor are included, individually or in any combination of two, within the same recombinant oncolytic virus, and constitute the only exogenous arming genes in the recombinant oncolytic virus.

In the present disclosure, a recombinant oncolytic virus is termed a ‘single-factor’ recombinant oncolytic virus if it can provide (i.e., express and produce) any one of the three factors of the invention (i.e., OX40L, IL-12, or PD-1 inhibitor) after infecting tumor cells. A recombinant oncolytic virus is termed a ‘two-factor’ recombinant oncolytic virus if it can provide two of these factors, or a ‘three-factor’ recombinant oncolytic virus if it can simultaneously provide all three factors.

Likewise, in the present disclosure, a recombinant oncolytic virus composition that provides (i.e., expresses and produces) OX40L and IL-12 among the three factors of the invention is termed a “two-factor” recombinant oncolytic virus composition; and a recombinant oncolytic virus composition that provides all three factors of the invention (i.e., OX40L, IL-12, and PD-1 inhibitor) is termed a “three-factor” recombinant oncolytic virus composition.

### II. The Present Invention

As demonstrated in the Examples, the inventors have surprisingly found that armed oncolytic viruses encoding trimeric OX40L and IL-12, or encoding trimeric OX40L, IL-12 and a PD-1 inhibitor, or a combination of armed oncolytic viruses encoding trimeric OX40L and IL-12 with a PD-1 inhibitor, can significantly induce the expression of MHC-I and -II molecules as well as co-stimulatory molecules such as CD80/CD86 on tumor cells of cancer patients, thereby transforming the tumor cells into antigen-presenting cells with APC characteristics.

Without being bound by any theory, during the induction phase of the anti-tumor immune response, tumor cells with APC characteristics, induced by infection with the armed oncolytic virus of the invention, present their own tumor antigens via MHC-I or MHC-II, and deliver costimulatory signals through co-stimulatory molecules CD80/CD86, thereby promoting T cell infiltration into the tumor tissue and enhancing the expansion and activation of tumor-infiltrating lymphocytes (TILs) within the tumor. Given that the number of tumor cells in a tumor tissue is significantly higher than that of professional antigen-presenting dendritic cells (DCs), restoring or enhancing the ability of tumor cells to directly present their own antigens also improves the recognition of tumor cells by anti-tumor TILs in the APC-deficient tumor tissue, thereby enhancing the clearance of tumors by TILs.

Building on these, the present invention provides a novel recombinant oncolytic virus composition, and the use of the composition and a method of using the composition for treatment of a cancer to enhance anti-tumor immunotherapy based on immune cells (especially tumor-infiltrating lymphocytes, i.e., TIL cells). The combination of the recombinant oncolytic virus composition—either as a three-factor composition alone or as a two-factor composition preferably combined with a PD-1 inhibitor—with an adoptive TIL therapy composition produces synergistic therapeutic efficacy that surpasses the efficacy of either composition used alone.

Various aspects of the present invention are described in detail below.

### Adoptive Cell Therapy Composition

In the present disclosure, the “adoptive cell therapy composition” preferably refers to an adoptive TIL cell therapy composition, i.e., a composition comprising tumor-infiltrating lymphocytes (TILs).

The application of adoptive TIL cells in cancer treatment typically involves transferring ex vivo-expanded TIL cells into a host to boost anti-cancer immunity. These transferred cells can be either autologous or allogeneic. Depending on the cancer type to be treated, in some instances, the TIL cells to be transferred may be sorted and enriched, or not sorted and enriched, for specific T cell subsets in vitro. Additionally, the TIL cells can be genetically unmodified or modified to transgenically express exogenous proteins, such as chimeric antigen receptors (CARs). Before TIL infusion, the patient may undergo myeloablative or non-myeloablative preconditioning with chemotherapy and/or radiation therapy.

In some preferred embodiments, autologous cells are used in the adoptive TIL cell therapy according to the present invention. In some embodiments, preferably, the adoptively transferred TIL lymphocytes are not subset-selected. In some other embodiments, the adoptively transferred TIL cells are subset-selected cells, such as CD8⁺ T cell-enriched or CD4⁺ T cell-enriched. In yet other embodiments, the adoptively transferred TIL cells are genetically unmodified.

In some embodiments, the adoptively transferred TIL cells are tumor-infiltrating lymphocytes isolated from the subject, which are capable of specifically recognizing and destroying the tumor cells in the cancer.

In the present disclosure, tumor-infiltrating lymphocytes (TILs) refer to lymphocytes that infiltrate tumors. TILs are generally categorized by one or more of the following biomarkers: CD4, CD8, TCRαβ, CD27, CD28, CD56, CCR7, CD45Ra, CD95, PD-1, and CD25. Preferably, the TILs are lymphocytes that can infiltrate solid tumors and exert anti-tumor effects.

In some embodiments, the isolated tumor-infiltrating lymphocytes are cultured ex vivo to a large number and, preferably intraperitoneally or, more preferably, intratumorally, re-infused into the patient. In some other embodiments, before re-infusing TIL cells, the subject receives one or more armed oncolytic viruses, particularly HSV-1 viruses, encoding trimeric OX40L, IL-12, and optionally a PD-1 inhibitor. Preferably, the two-factor or three-factor recombinant oncolytic virus composition of the invention is administered. When using the two-factor recombinant oncolytic virus composition, one or more doses of a PD-1 inhibitor may be administered during the treatment process as appropriate.

The isolation and ex vivo expansion of TILs for use in the adoptive therapy of the present invention can be carried out by any of the methods known in the field. As an example, TILs can be expanded ex vivo by culturing isolated TIL cells in a cell culture medium containing cytokines IL-2, IL-7, IL-15, and an anti-CD3 antibody, followed by a rapid expansion. This approach is preferred for TIL expansion due to its speed and efficiency. In the first stage of TIL expansion, the initial concentrations of IL-2/IL-7/IL-15 cytokines in the cell culture medium may be about 5 ng/ml, and the anti-CD3 antibody (e.g., OKT-3 antibody) may be about 30 ng/ml.

The ex vivo expansion of TILs may involve the use of feeder cells, or alternatively, the isolated TILs can be directly passaged and expanded. For instance, after digesting a tumor sample from a subject into single cells, the cells can be plated in 24-well plates and cultured for a period, such as 24 hours. Subsequently, the cells in the supernatant can be collected for further isolation and expansion. Compared to using feeder cells, the direct expansion method is more convenient and ensures uniform cell distribution across wells, which helps prevent cell death caused by insufficient contact with culture medium.

In one embodiment of any methods and/uses according to the present invention, the methods and/or uses include a step of ex vivo expansion of TILs isolated from the subject, comprising:
(a) obtaining a first TIL population from tumor tissue resected from the patient;
(b) expanding the first TIL population in a cell culture medium containing IL-2, IL-7, IL-15, and the OKT-3 antibody to obtain a second TIL population;
(c) rapidly expanding the second TIL population in a cell culture medium containing a high concentration of IL-2 to obtain a third TIL population.

The TIL isolation in step (a) may include: digesting and dispersing tumor tissue from the tumor subject into single cells, preferably using a digestion buffer containing collagenase IV, hyaluronidase II, and DNase I Type IV for tumor tissue digestion; separating TIL cells by discontinuous density gradient centrifugation (e.g., a discontinuous density gradient of a standard isotonic Percoll (SIP) solution).

The first-stage TIL expansion in step (b) is preferably conducted under a low concentration of IL-2. For instance, the expansion may include: culturing the isolated TIL cells in a medium containing IL-2 (about 5 ng/ml or about 100 IU/ml), IL-7 (about 5 ng/ml), IL-15 (about 5 ng/ml), and an anti-CD3 antibody (about 30 ng/ml).

The rapid expansion in step (c) is preferably carried out using about 3000 IU/ml of IL-2.

Optionally, after step (c), the process includes co-culturing the TIL cells with cancer cells, particularly those stimulated with a DEC cocktail containing dacarbazine, TNF-α, and IFN-γ, to activate the TILs; and optionally, the isolated and expanded TIL cells undergo or do not undergo subset enrichment. Preferably, after expansion, the TIL cells are re-infused into the subject directly without subset enrichment.

The expanded TIL cells may be administered directly to the subject. Alternatively, the TIL cells can be cryopreserved at about −150 to −60° C. General methods for cryopreserving TIL cells are known in the art.

In some embodiments, the adoptively transferred TIL cells are included in adoptive cell therapy compositions. The compositions may comprise pharmaceutically acceptable carriers, buffers, excipients, adjuvants, additives, antimicrobials, fillers, stabilizers and/or viscosity enhancers, and/or any other components typically present in the corresponding adoptive cell therapy products. Suitable reagents and methods for formulating adoptive cell therapy products are known in the art. The adoptively transferred TIL cells can be formulated into any suitable composition form for administration, e.g., solid, semi-solid, or liquid form. The formulations can be selected from, but are not limited to, solutions, emulsions, suspensions, tablets, and capsules. In a preferred embodiment, the formulation is one suitable for intraperitoneal administration of adoptive TIL cells, and more preferably, for intratumoral injection.

### Recombinant Oncolytic Viruses and Their Compositions

The recombinant oncolytic viruses according to the present invention can be any oncolytic virus suitable for humans or animals, including but not limited to, herpes simplex virus, reovirus, vaccinia virus, particularly herpes simplex virus type 1 (HSV-1). For example, a clinical isolate of HSV-1 can be engineered to produce the recombinant oncolytic viruses of the present invention. The engineering includes inserting a polynucleotide(s) encoding one, two, or three of trimeric OX40L, IL-12, and PD-1 inhibitor into the HSV-1 genome. According to the present invention, the engineered oncolytic virus is also referred to as a recombinant oncolytic virus.

In one embodiment, the present invention thus provides at least one recombinant oncolytic virus and a recombinant oncolytic virus composition comprising it, wherein the at least one recombinant oncolytic virus comprises polynucleotides encoding trimeric OX40L, IL-12, and optionally PD-1 inhibitor in its genome.

In one embodiment, the at least one recombinant oncolytic virus is one recombinant oncolytic virus comprising polynucleotides encoding trimeric OX40L, IL-12, and optionally a PD-1 inhibitor in its genome, preferably, the recombinant oncolytic virus is a two-factor recombinant oncolytic virus encoding trimeric OX40L and IL-12. In another embodiment, the at least one recombinant oncolytic virus consists of more than one recombinant oncolytic virus, e.g., two or three recombinant oncolytic viruses, wherein, upon administration of the more than one different recombinant oncolytic viruses to the subject, trimeric OX40L and IL-12, or preferably trimeric OX40L, IL-12, and a PD-1 inhibitor, are recombinantly expressed in the subject's tumor cells. In the embodiments involving two recombinant oncolytic viruses, preferably, the first recombinant oncolytic virus comprises polynucleotides encoding trimeric OX40L and PD-1 inhibitor in its genome; the second recombinant oncolytic virus comprises polynucleotides encoding IL-12 and PD-1 inhibitor in its genome. In the embodiments involving three recombinant oncolytic viruses, preferably, the first recombinant oncolytic virus comprises a polynucleotide encoding trimeric OX40L in its genome; the second recombinant oncolytic virus comprises a polynucleotide encoding IL-12 in its genome; the third recombinant oncolytic virus comprises a polynucleotide encoding PD-1 inhibitor in its genome. In embodiments of any methods and uses of the present invention where a PD-1 inhibitor is provided through a recombinant oncolytic virus of the present invention, it may be considered to separately administer a PD-1 inhibitor as an alternative. Such alternative embodiments are within the scope of the present invention.

It is well-known in the art which locations in the HSV-1 genome allow for the insertion of exogenous polynucleotides without affecting the replication and infection functions of the virus. Suitable insertion sites for inserting exogenous polynucleotides into the HSV-1 genome can be selected as needed. In the present disclosure, when two or more exogenous arming genes are inserted into the same virus genome, it is preferred that the exogenous arming genes are inserted at different locations in the virus genome. As used herein, in respect of exogenous arming genes inserted into the virus genome, being located at or inserted into “different genomic locus” means that these genes are separated from each other by at least one or more virus genes. Therefore, in embodiments where a single recombinant oncolytic virus is used to provide OX40L, IL-12, and PD-1 inhibitor, it is preferred that the OX40L, IL-12, and PD-1 inhibitor are inserted at different genomic loci. In embodiments where a first and a second recombinant oncolytic virus are used, it is preferred that the first recombinant oncolytic virus comprises polynucleotides encoding OX40L and PD-1 inhibitor inserted at different genomic loci; and the second recombinant oncolytic virus comprises polynucleotides encoding IL-12 and PD-1 inhibitor inserted at different genomic loci.

Those skilled in the art understand that when engineering HSV-1, it is preferable to minimize unintended changes to the viral genome, and insertion sites for exogenous genes should ideally not affect viral growth or pathology. The sites in the HSV-1 genome suitable for insertion of exogenous genes include, but are not limited to, the ICP34.5 gene locus and the intergenic regions between UL3/UL4, UL50/UL51, US1/US2, and UL26/UL27. For insertions into intergenic regions, the insertion of exogenous genes should ideally not disrupt the transcription of genes flanking the insertion site.

To insert exogenous genes into the HSV-1 genome, homologous recombination between the viral genome and a plasmid containing the exogenous gene can be performed in mammalian cells. One possible method is to co-transfect mammalian cells with the plasmid and the isolated viral genomic DNA. Alternatively, the transfection-infection method can be used, where the virus genome is provided through infection. Specifically, cells transfected with the plasmid are supplied with the viral genome by HSV-1 infection. In such approach, several rounds (e.g., 3-4 rounds) of virus plaque purification are conducted after infection, to screen for recombinant viruses with the correctly inserted exogenous genes. Mammalian cells suitable for constructing recombinant oncolytic viruses include, but are not limited to, Vero cells and 293 cells.

The transfection-infection method used for constructing recombinant HSV-1 oncolytic viruses can be further combined with CRISPR/Cas9 genome editing methods, TALEN genome editing methods, or ZFN genome editing methods.

The at least one recombinant oncolytic viruses of the present invention express trimeric OX40L and IL-12, or preferably trimeric OX40L, IL-12, and PD-1 inhibitor, after infecting tumor cells. Therefore, in some preferred embodiments, the exogenous polynucleotides encoding OX40L, IL-12, and optionally a PD-1 inhibitor are inserted into the HSV-1 genome as expression cassettes. Preferably, the expression cassettes include a promoter and a terminator operably linked to the exogenous polynucleotides. Any promoter that can initiate the expression of the exogenous polynucleotides in tumor cells can be used, such as promoters from mammalian cells or their viruses, e.g., the CMV promoter. Any terminator sequence that can signal the end of the expression of the exogenous polynucleotides in tumor cells can be used, e.g., terminator sequences from mammalian cells or their viruses, such as a poly(A) signal sequence, preferably selected from the SV40 late poly(A) sequence, rabbit β-globin poly(A) sequence, bovine growth hormone poly(A) sequence, more preferably the SV40 poly(A) sequence. In a preferred embodiment, the expression cassettes containing the exogenous polynucleotides encoding OX40L, IL-12, and the PD-1 inhibitor each include a CMV promoter and, more preferably, an SV40 poly(A) sequence functionally linked to the encoding polynucleotide.

In addition to the insertion of OX40L, IL-12, and/or a PD-1 inhibitor, additional genomic modifications may be introduced into the recombinant oncolytic virus(s) to enhance its ability to selectively replicate in tumor cells and/or increase antigen presentation by tumor cells infected with the virus(s). In one embodiment, the ICP34.5 (either single copy or double copies) and ICP47 genes in the genome of the recombinant HSV-1 oncolytic virus are knocked out. Preferably, the virus is an HSV-1 virus with deletions of ICP47 and both copies of ICP34.5.

In some embodiments, the factors of the present invention, OX40L, IL-12, and/or PD-1 inhibitor are preferably provided by a two-factor recombinant oncolytic virus. Thus, in one aspect, the present disclosure provides a two-factor recombinant oncolytic virus, wherein the recombinant oncolytic virus is an HSV-1 that comprises (and preferably only comprises) in its genome two exogenous arming genes, selected from the group consisting of:
(a) a polynucleotide encoding trimeric OX40L and a polynucleotide encoding a PD-1 inhibitor, preferably, the OX40L encoding nucleic acid is inserted in two copies at the two ICP34.5 loci of the virus genome, and the PD-1 inhibitor encoding nucleic acid is inserted in the UL26/UL27 intergenic region of the virus genome;
(b) a polynucleotide encoding IL-12 and a polynucleotide encoding a PD-1 inhibitor, preferably, the IL-12 encoding nucleic acid is inserted in two copies at the two ICP34.5 loci of the virus genome, and the PD-1 inhibitor encoding nucleic acid is inserted in the UL26/UL27 intergenic region of the virus genome; and
(c) a polynucleotide encoding trimeric OX40L and a polynucleotide encoding IL-12, preferably, the OX40L encoding nucleic acid is inserted in two copies at the two ICP34.5 loci of the virus genome, and the IL-12 encoding nucleic acid is inserted in the UL26/UL27 intergenic region of the virus genome.

In some embodiments, the present disclosure provides a recombinant oncolytic virus composition comprising at least one recombinant oncolytic viruses of the present invention. In this context, when the recombinant oncolytic virus composition of the present invention comprises two or more recombinant oncolytic viruses, the term “recombinant oncolytic virus composition” can be used interchangeably with “recombinant oncolytic virus combination”, and refers to a composition or a combination product comprising the at least one recombinant oncolytic virus. In the recombinant oncolytic virus composition of the present invention, the at least one recombinant oncolytic virus can be prepared in a single formulation. Alternatively, if the composition comprises two or more recombinant oncolytic viruses, each virus, or any combination of two or more of them, can be formulated either together in the same formulation or separately in different formulations.

In one embodiment of the composition, the recombinant oncolytic virus of the present invention is an HSV-1 virus with knockout of ICP47 and knockout of double copies of ICP34.5. In another embodiment, the recombinant oncolytic virus comprises exogenous polynucleotides encoding any one, any two, or all three of trimeric OX40L, IL-12, and a PD-1 inhibitor in its genome. In one preferred embodiment, the exogenous polynucleotide encoding OX40L is inserted at one or preferably both of the two ICP34.5 loci in the virus genome, wherein the insertion results in a knockout of the ICP34.5 gene at the insertion site(s). In another preferred embodiment, the exogenous polynucleotide encoding IL-12 is inserted at one or preferably both of the two ICP34.5 loci in the virus genome, resulting in a knockout of the ICP34.5 gene at the insertion site(s). In yet another preferred embodiment, the exogenous polynucleotide encoding the PD-1 inhibitor is inserted in the intergenic region between UL26 and UL27 in the virus genome. Preferably, the exogenous nucleic acids encoding the OX40L, IL-12, and PD-1 inhibitor are functionally linked to a CMV promoter. More preferably, the exogenous polynucleotides encoding the OX40L, IL-12, and PD-1 inhibitor are further functionally linked to a transcription terminator, such as a SV40 poly(A) sequence.

In one embodiment, the recombinant oncolytic virus composition comprises only one recombinant oncolytic virus, wherein the recombinant oncolytic virus comprises exogenous polynucleotides encoding both trimeric OX40L and IL-12 in its genome. In one embodiment, the insertion sites for the exogenous polynucleotides are selected from the group consisting of: ICP34.5, between UL3 and UL4, or between UL26 and UL27. In one preferred embodiment, the nucleic acid coding for trimeric OX40L is inserted at one or preferably both of the two ICP34.5 loci; and the nucleic acid coding for IL-12 is preferably inserted between UL26 and UL27.

In one embodiment, the recombinant oncolytic virus composition comprises a first oncolytic virus and a second oncolytic virus. In one embodiment, the first and second oncolytic viruses are ICP47 knockout and ICP34.5 double-copy knockout HSV-1 viruses. In another embodiment, the first virus comprises an OX40L coding polynucleotide inserted at one or preferably both of the two ICP34.5 loci in the virus genome; and the second virus comprises an IL-12 coding polynucleotide inserted at one or preferably both of the two ICP34.5 loci in the virus genome. In yet another embodiment, the first virus and the second virus each also comprise an exogenous polynucleotide encoding a PD-1 inhibitor, wherein the exogenous polynucleotide is inserted in the intergenic region between UL26 and UL27 of the recombinant oncolytic virus genome. Preferably, the exogenous polynucleotides encoding the OX40L, IL-12, and PD-1 inhibitor are functionally linked to a CMV promoter. More preferably, the exogenous polynucleotides encoding the OX40L, IL-12, and PD-1 inhibitor are further functionally linked to a transcription terminator, such as a SV40 poly(A) sequence.

The OX40L encoding nucleic acid used according to the present invention can be any polynucleotide that expresses a functionally trimeric OX40L polypeptide on the surface of tumor cells upon virus infection. In a preferred embodiment, the OX40L encoding nucleic acid encodes a protein comprising the amino acid sequence of SEQ ID NO: 18, or a variant thereof having an amino acid sequence with at least 90%, 91%, 92%, 93%, 94%, 95%, 96%, 97%, 98%, 99% identity, particularly a conservative amino acid substitution variant. In some other embodiments, the OX40L encoding nucleic acid comprises the nucleotide sequence of SEQ ID NO: 3.

The IL-12 encoding nucleic acid used according to the present invention can be any polynucleotide that expresses and secretes a functional IL-12 protein from tumor cells upon virus infection. In a preferred embodiment, the IL-12 encoding nucleic acid encodes a heterodimeric protein comprising or consisting of an IL-12α polypeptide and an IL-12β polypeptide, wherein the IL-12α polypeptide comprises or consists of the amino acid sequence of SEQ ID NO: 17, or comprises or consists of an amino acid sequence having at least 90%, 91%, 92%, 93%, 94%, 95%, 96%, 97%, 98%, 99% identity thereto; and the IL-12β polypeptide comprises or consists of the amino acid sequence of SEQ ID NO: 16, or comprises or consists of an amino acid sequence having at least 90%, 91%, 92%, 93%, 94%, 95%, 96%, 97%, 98%, 99% identity thereto. More preferably, the IL-12 consists of an IL-12α polypeptide comprising or consisting of the amino acid sequence of SEQ ID NO: 17, and an IL-12β polypeptide comprising or consisting of the amino acid sequence of SEQ ID NO: 18. In some other preferred embodiments, the IL-12 encoding nucleic acid comprises the nucleotide sequences of SEQ ID NO: 1 and SEQ ID NO: 2.

The PD-1 inhibitor encoding nucleic acid used according to the present invention can be any polynucleotide that expresses and secretes a functional PD-1 inhibitor polypeptide from tumor cells upon virus infection. Any of various PD-1 inhibitors known in the art can be used in the present invention. Preferably, the PD-1 inhibitor is an anti-PD-1 antibody, more preferably an anti-PD-1 scFv antibody. In some preferred embodiments, the anti-PD-1 scFv antibody comprises a VH and a VL, wherein the VH comprises the HCDR1 amino acid sequence of SEQ ID NO: 22, the HCDR2 amino acid sequence of SEQ ID NO: 23, and the HCDR3 amino acid sequence of SEQ ID NO: 24; and the VL comprises the LCDRI amino acid sequence of SEQ ID NO: 25, the LCDR2 amino acid sequence of SEQ ID NO: 26, and the LCDR3 amino acid sequence of SEQ ID NO: 27. Preferably, the anti-PD-1 scFv antibody comprises the VH amino acid sequence of SEQ ID NO: 20 and the VL amino acid sequence of SEQ ID NO: 21. More preferably, the scFv antibody comprises or consists of the amino acid sequence of SEQ ID NO: 19.

In the recombinant oncolytic virus composition of the present invention, preferably, each of the at least one recombinant oncolytic virus contained therein comprises 1-4 exogenous arming genes, preferably no more than 3, more preferably no more than 2 exogenous arming genes. More preferably, the at least one recombinant oncolytic virus comprises a total of no more than 10, preferably 2, 3, 4, 5, or 6, more preferably 4 or 3 or 2 exogenous arming genes. Therefore, in some embodiments, the two-factor recombinant oncolytic virus composition of the present invention, which provides trimeric OX40L and IL-12, or the three-factor composition, which provides trimeric OX40L, IL-12, and a PD-1 inhibitor, may also provide one or more additional exogenous arming genes that are not the factors of the present invention (i.e., other than trimeric OX40L, IL-12, and a PD-1 inhibitor). However, preferably, the compositions do not provide other exogenous arming genes.

In a further aspect, the present invention also provides pharmaceutical compositions or pharmaceutical preparations comprising at least one recombinant oncolytic viruses of the present invention, such as one, two or three recombinant oncolytic viruses, which express trimeric OX40L, IL-12 and optionally PD-1 inhibitor after infecting tumor cells. In addition to the recombinant oncolytic viruses, the pharmaceutical combinations or pharmaceutical compositions may also comprise other therapeutic active agents, in particular the adoptive TIL cells of the present invention, and/or pharmaceutically acceptable carriers. Suitable examples of pharmaceutically acceptable carriers include, but are not limited to, buffers, excipients, adjuvants, additives, preservatives, fillers, stabilizers. Suitable other therapeutic active agents may include, but are not limited to, immunomodulators, anticancer drugs, radiation drugs, chemotherapeutic drugs, etc.

### Administration

The recombinant oncolytic virus compositions or the pharmaceutical compositions or pharmaceutical preparations comprising the recombinant oncolytic viruses of the present invention can be administered to a subject, preferably a human cancer patient.

Any suitable route and mode of administration for delivering the recombinant oncolytic viruses or their compositions, and/or the adoptive TIL cells or adoptive cell therapy compositions of the present invention, to a subject to achieve their intended function are encompassed within the scope of the present invention. The administration routes may include, but are not limited to, oral, intranasal, parenteral (such as intravenous, intramuscular, intradermal, intraperitoneal, or subcutaneous), rectal, intrathecal, intratumoral, or topical.

As an example, the recombinant oncolytic viruses or compositions of the present invention are administered to a subject via conventional administration methods for oncolytic HSV-1 viruses. The adoptive TIL cells or adoptive cell therapy compositions of the present invention are administered to a subject via TIL administration methods conventional in adoptive cell therapy. The administration route will depend on the active ingredients to be administered, the dosage form or modality of the pharmaceutical compositions, the type of cancer, the tumor site, the patient's condition, comorbidities and other factors.

In one embodiment of the present invention, the adoptive T cell therapy composition and the recombinant oncolytic virus composition are co-administered. This administration can occur concurrently, simultaneously, or sequentially in any order. Preferably, the adoptive T cells and the recombinant oncolytic virus(es) are provided in separate products or compositions. Administration can be a single event, or involve multiple administrations, with any time intervals between each administration. For example, depending on factors such as the patient and the type of cancer, the interval ranges from 1 minute to 4 weeks, such as 1-10 days. Alternatively, administration can be continuous over multiple days.

The frequency of administrations of the adoptive T cell compositions may be the same as or different from the frequency of administrations of the recombinant oncolytic virus compositions. Preferably, the recombinant oncolytic virus composition is administered a period of time prior to the administration of the adoptive T cell composition, so that the recombinant oncolytic virus(s) infect the tumor cells and express trimeric OX40L and IL-12, or preferably express trimeric OX40L, IL-12 and the PD-1 inhibitor, before the TILs are transferred.

The administration of the oncolytic virus may be via intratumoral, intra-arterial, intravenous, intraperitoneal, intrapleural, intracavitary, or oral administration. It may also be a combination of any administration modes. Preferably, the oncolytic virus is administered intratumorally. The adoptive T cell therapy composition may be administered intravenously, intraperitoneally or intratumorally. In one embodiment, the adoptive TIL cells are administered intravenously, and the recombinant oncolytic virus(es) is administered intratumorally and/or intravenously. In another embodiment, both the adoptive TIL cells and the recombinant oncolytic virus(es) are administered intratumorally.

### Methods and Uses

The present invention relates to methods of treating cancer, improving antigen presentation of tumor cells, and/or improving the efficacy of adoptive TIL cell therapy in a subject using the recombinant oncolytic virus compositions of the invention, optionally in combination with the adoptive cell therapy compositions of the invention. As used herein, a subject includes but is not limited to a human or mammal, particularly a human patient.

The methods of the invention can be used for any cancer or tumor subject, particularly solid tumors, such as malignant solid tumors, primary solid tumors, and metastatic solid tumors. In one embodiment, the tumor tissue of the cancer comprises tumor-infiltrating lymphocytes. In other embodiments, the tumor tissue of the cancer has a low degree of lymphocyte infiltration.

Examples of solid tumors that can be treated by the methods of the invention include, but are not limited to: head and neck cancer, such as oral cancer; squamous cell carcinoma; rectal adenocarcinoma, glioma, melanoma, pancreatic cancer, uterine/ovarian cancer, cervical cancer, prostate cancer, lung cancer, non-small cell lung cancer, small cell lung cancer, breast cancer, bladder cancer, renal cell carcinoma, and hepatocellular carcinoma, esophageal cancer, eye cancer, gastrointestinal cancers, and their metastases.

In some embodiments, the solid tumor to be treated is selected from head and neck cancer, laryngeal cancer, hypopharyngeal cancer, oral cancer (e.g., lip cancer, gingival cancer, buccal cancer, and tongue cancer). Preferably, the solid tumor is squamous cell carcinoma. In some embodiments, the solid tumor to be treated is selected from colorectal cancer and its metastases. In some instances, the solid tumor exhibits a low degree of infiltration.

Without being bound by any theory, the recombinant oncolytic virus compositions of the invention can, through intratumoral injection, convert tumor cells into antigen-presenting cells, induce and/or enhance antigen presentation of tumor cells; and optionally achieve one or more of the following: (i) guide/recruit tumor-specific T cells to the tumor site; (ii) reduce tumor tolerance by increasing danger signals; and (iii) induce TIL cell proliferation within the tumor tissue by improving its immunosuppressive environment. Consequently, the recombinant oncolytic virus compositions of the invention are advantageous for promoting the recruitment, maintenance, expansion, and/or activation of tumor-infiltrating lymphocytes (including both endogenous TILs and those transferred during adoptive cell therapy) at the tumor site, and thereby enhancing anti-tumor efficacy.

Thus, in one aspect, the present invention provides a method for treating a cancer subject, or a method for improving tumor infiltrating lymphocyte (TIL) adoptive therapy in a cancer subject, the method comprising administering to the subject:
(a) a recombinant oncolytic virus composition, the composition comprising at least one recombinant oncolytic virus, wherein the at least one recombinant oncolytic virus, upon infecting the tumor cells of the subject, expresses exogenous trimeric OX40L and IL-12 or preferably expresses exogenous trimeric OX40L, IL-12 and a PD-1 inhibitor,
preferably, the method further comprises administering
(b) an adoptive cell therapy composition, the composition comprising tumor infiltrating lymphocytes (TILs), wherein the TILs are derived from the same subject from whom the tumor cells originate,
wherein the at least one recombinant oncolytic virus is herpes simplex virus HSV-1.

In another aspect, the present invention provides a method for converting tumor cells into antigen presenting cells (APCs) in a subject, the method comprising administering:
(a) a recombinant oncolytic virus composition of the invention, the composition comprising at least one recombinant oncolytic virus, wherein the at least one recombinant oncolytic virus, upon infecting the tumor cells of the subject, expresses exogenous trimeric OX40L and IL-12 or preferably expresses trimeric OX40L, IL-12 and a PD-1 inhibitor,
preferably, the method further comprises administering
(b) an adoptive cell therapy composition of the invention, the composition comprising tumor infiltrating lymphocytes (TILs), wherein the TILs are derived from the same subject from whom the tumor cells originate,
wherein the at least one recombinant oncolytic virus is herpes simplex virus HSV-1.

In some embodiments, the present invention provides a method for treating a cancer patient, or a method for improving adoptive cell therapy in a cancer patient, the method comprising administering:
a) a recombinant oncolytic virus composition, or
b) a recombinant oncolytic virus composition with a PD-1 inhibitor, or
c) (a) or (b) with an adoptive cell therapy composition,
wherein the recombinant oncolytic virus composition comprises at least one (e.g. one or two or three, preferably two) recombinant oncolytic virus, wherein the at least one recombinant oncolytic virus, upon infecting the subject's tumor cells, expresses exogenous trimeric OX40L and IL-12 and optionally a PD-1 inhibitor,
wherein the adoptive cell therapy composition comprises tumor infiltrating lymphocytes (TILs),
wherein preferably the TILs are derived from the same subject from whom the tumor cells originate.

In some preferred embodiments, the methods comprise administering a two-factor recombinant oncolytic virus composition of the invention alone.

In some other preferred embodiments, the methods comprise administering a three-factor recombinant oncolytic virus composition of the invention alone.

In some other preferred embodiments, the methods comprise administering a combination of a two-factor recombinant oncolytic virus composition of the invention with:
(i) a PD-1 inhibitor; or
(ii) an adoptive cell therapy composition; or
(iii) (i) and (ii).

In yet other preferred embodiments, the methods comprise administering a combination of a three-factor recombinant oncolytic virus composition of the invention with an adoptive cell therapy composition.

In the methods according to these embodiments, preferably, the two-factor recombinant oncolytic virus composition of the invention comprises or consists of a single recombinant oncolytic virus having both a trimeric OX40L encoding nucleic acid and an IL-12 encoding nucleic acid in its genome; or comprises or consists of a first recombinant oncolytic virus having a trimeric OX40L encoding nucleic acid in its genome and a second recombinant oncolytic virus having an IL-12 encoding nucleic acid in its genome.

In the methods according to these embodiments, preferably, the three-factor recombinant oncolytic virus composition of the invention comprises or consists of a first recombinant oncolytic virus having a trimeric OX40L encoding nucleic acid and a PD-1 inhibitor encoding nucleic acid in its genome, and a second recombinant oncolytic virus having an IL-12 encoding nucleic acid and a PD-1 inhibitor encoding nucleic acid in its genome.

In some preferred embodiments of any of the above methods, the virus is a HSV-1, wherein the ICP34.5 and ICP47 genes in the viral genome are knocked out, preferably the virus is an HSV-1 virus with deletion of ICP47 and deletion of double copies of ICP34.5.

In one embodiment of the above methods, the administration of at least one recombinant oncolytic virus comprises: administering a single recombinant oncolytic virus having polynucleotides encoding both trimeric OX40L and IL-12 in its genome. Preferably, in the genome of said oncolytic virus, the nucleic acid encoding trimeric OX40L is inserted into both of the two ICP34.5 loci, and the nucleic acids encoding IL-12x and IL-12β of IL-12, linked by an IRES2 sequence, are inserted between UL26 and UL27.

In one embodiment of any of the above methods, the administering of at least one recombinant oncolytic virus comprises: administering a first recombinant oncolytic virus and a second recombinant oncolytic virus, wherein: the first recombinant oncolytic virus encodes trimeric OX40L and a PD-1 inhibitor in its genome; the second recombinant oncolytic virus encodes IL-12 and a PD-1 inhibitor in its genome. Preferably, the first virus comprises the OX40L encoding nucleic acid inserted into one or preferably both of the two ICP34.5 loci of the viral genome; the second virus comprises the IL-12 encoding nucleic acid inserted into one or preferably both of the two ICP34.5 loci of the viral genome. More preferably, the first virus and the second virus each further comprise a nucleic acid encoding a PD-1 inhibitor, preferably the PD-1 inhibitor encoding nucleic acid is inserted into the intergenic region between UL26 and UL27 of the recombinant oncolytic virus genome, preferably the PD-1 inhibitor is an anti-PD-1 single chain scFv antibody.

In any of the above methods, preferably the subject is a human cancer patient.

In one embodiment of any of the above methods, the adoptive cell therapy composition comprising the TILs of the invention and the recombinant oncolytic virus composition comprising at least one recombinant oncolytic virus of the invention, e.g. the two-factor or three-factor recombinant oncolytic virus composition, are administered intratumorally to the subject sequentially. In another embodiment, the recombinant oncolytic virus composition and the adoptive cell therapy composition are administered simultaneously or sequentially in any order, preferably the oncolytic virus composition is administered before the adoptive cell therapy composition, more preferably there is an interval of 10 hours to 72 hours, such as 24-48 hours, e.g. about 36 hours or 48 hours, between administration of the oncolytic virus composition and administration of the adoptive cell therapy composition. In embodiments involving administration of the two-factor recombinant oncolytic virus composition, preferably a separate PD-1 inhibitor is co-administered. For example, the administration of the PD-1 inhibitor is made before, simultaneously or after the oncolytic virus composition and adoptive cell therapy composition. The PD-1 inhibitor may be administered in one or multiple doses, preferably multiple doses, during the treatment course, e.g. administered at intervals and for a period of time, such as days, weeks, months or longer, depending on the condition of the disease.

In one embodiment of any of the above methods, the recombinant oncolytic virus composition of the invention comprising a first and second oncolytic virus is administered intratumorally to the subject. Preferably, a first oncolytic virus expressing OX40L and a PD-1 inhibitor and a second oncolytic virus expressing IL-12 and a PD-1 inhibitor are administered intratumorally to the subject. Preferably, the first and second virus are administered at a ratio of 1:1 to 3:1, such as about 1.5:1, about 2:1, and about 2.5:1. The first and second oncolytic viruses may be formulated in either separate or combined pharmaceutical compositions.

In one embodiment of any of the above methods, the methods further comprise administering to the subject an IL-2 protein, e.g. a super-IL-2 protein, preferably by intraperitoneal administration. Preferably, the IL-2 protein is administered after administration of the oncolytic virus and/or TILs. Regarding super-IL-2, see e.g. Aron M Levin et al., Exploiting a natural conformational switch to engineer an interleukin-2 ‘superkine’, Nature, 2012 Mar. 25; 484 (7395): 529-33. doi: 10.1038/nature10975.

In one embodiment of any of the above methods, the methods of the invention result in one or more of the following:
Increased expression of antigen presenting molecules on the surface of tumor cells, preferably the antigen presenting molecules are selected from one or more of: HLA-A/B/C, HLA-DR/DP/DQ, CD80, CD83 and CD86; more preferably selected from one or more of: HLA-C, HLA-DRB1, CD80, CD83 and CD86; more preferably, CD86;
and/or
Enhanced activation and/or expansion of TILs in the subject's tumor tissue;
Upregulation of IFN-γ levels in the subject's tumor tissue;
Inhibition of tumor cell growth or reduction in tumor volume;
Improved survival of the subject.

The combination of the recombinant oncolytic virus composition (optionally with a PD-1 inhibitor) and the adoptive TIL therapy composition of the invention offers the advantages, including enhanced cancer treatment efficacy and reduced side effects. The methods of the invention allow for a reduction in the number of T cells needed for TIL adoptive transfer; shorten the ex vivo expansion time for TILs to align with the patient's optimal therapeutic window; and/or decrease the amount of IL-2 required to maintain, expand, and activate the adoptive TILs in vivo, thereby avoiding the side effects associated with high-dose IL-2, such as toxicity or damage to healthy tissues.

In one embodiment of the above methods, therefore, a reduced dose of TILs is administered compared to when TILs are used alone. Preferably, the methods also involve administering a reduced dose of IL-2 to maintain the in vivo expansion and activation of the adoptively transferred TILs.

In another aspect, the present disclosure also provides the use of or a method of using the recombinant oncolytic viruses and compositions thereof of the invention in a cancer subject, preferably wherein the recombinant oncolytic viruses and compositions thereof are administered to the subject in combination with the adoptive cell therapy composition of the invention. In some embodiments of said use or method, a PD-1 inhibitor is also co-administered, particularly when the recombinant oncolytic virus composition is the two-factor recombinant oncolytic virus composition.

In some embodiments, the present disclosure provides a method for converting tumor cells into antigen-presenting cells (APCs) or enhancing the activation of tumor-infiltrating lymphocytes (TILs). The method comprises infecting tumor cells with the oncolytic virus composition of the invention and contacting the infected tumor cells with TILs, wherein said TILs are from the same cancer subject as said tumor cells.

In some embodiments, the method is an ex vivo method, wherein the infecting and contacting are performed ex vivo, preferably the first and second oncolytic viruses infect the tumor cells at a multiplicity of infection (MOI) of at least 0.01; more preferably the TILs are co-cultured with the oncolytic virus-infected tumor cells at a ratio of at least 1:1, e.g. 1:2, 1:5.

Preferably, in some embodiments, the infecting and contacting occurs in vivo. In these embodiments, preferably the method further comprises the steps of isolating tumor infiltrating lymphocytes from the tumor tissue of the subject before or after infecting the tumor cells of the subject with the oncolytic virus, and infusing the isolated TILs back to the subject.

In some preferred embodiments, the method comprises isolating TILs from the subject before administering the oncolytic virus, and administering the isolated TILs in combination with the oncolytic virus to the subject.

In another preferred embodiment, the method comprises isolating tumor infiltrating lymphocytes from the tumor tissue of the subject after administering the oncolytic virus, and administering the isolated TIL cells back to the tumor subject.

In some other embodiments, the method comprises administering, preferably intratumorally, to the subject the oncolytic virus composition in combination with TILs isolated from the subject.

Preferably, the methods of the invention enhance expression of antigen presenting molecules on the surface of tumor cells, and/or increase the ability of tumor cells to present their own tumor antigens to TILs. In some embodiments, by the methods of the invention, the tumor cells infected with the oncolytic virus stimulate and induce expansion of the tumor infiltrating lymphocytes they contact. More preferably, the method increases the proportion of activated TIL cells, enhances the anti-tumor effect of the activated TILs, and/or promotes expansion of the TIL cells. Further preferably, upon contacting the oncolytic virus-infected tumor cells, the TILs express increased IFN-γ.

In some embodiments, the present disclosure provides the uses of the recombinant oncolytic viruses or compositions thereof of the invention, optionally together with one or both of the adoptive cell therapy composition comprising tumor infiltrating lymphocytes and a PD-1 inhibitor, in the preparation of a medicament for treating a tumor patient or for improving tumor infiltrating lymphocyte (TIL) adoptive therapy in a tumor patient, or in the preparation of a medicament, pharmaceutical composition, kit or combination product for use in any of the above methods of the invention.

### Combination Product

In another aspect, the invention provides a combination product comprising:
(a) a recombinant oncolytic virus composition of the invention; and
(b) one or both of an adoptive cell therapy composition comprising tumor infiltrating lymphocytes of the invention and a PD-1 inhibitor of the invention.

The combination product may further comprise reagents, compositions, and/or materials beneficial for practicing any of the above methods of the invention. For example, it may further comprise reagents for isolating TILs from tumor tissue, culture media and reagents for ex vivo expansion of TILs, and/or devices for TIL refusion; or materials and/or devices related to the preparation, storage and/or administration of the recombinant oncolytic virus composition.

In a preferred embodiment, the invention provides a combination product comprising: a two-factor recombinant oncolytic virus of the invention or a recombinant oncolytic virus composition of the invention (preferably a two-factor or three-factor recombinant oncolytic virus composition) with
(a) a PD-1 inhibitor; or
(b) an adoptive cell therapy composition; or
(c) a combination of (a) and (b).

Preferably, when the pharmaceutical combination or the combination product comprises a PD-1 inhibitor, the recombinant oncolytic virus composition is a two-factor recombinant oncolytic virus composition.

In any of the above embodiments of the invention, as understood by those skilled in the art, the adoptive TIL cells may be in place of an adoptive cell therapy composition comprising lymphocytes modified with a T-cell receptor or lymphocytes modified with a chimeric antigen receptor.

In any of the above embodiments of the invention, the subject may be a mammal, particularly a human.

In any of the above embodiments of the invention, the treatment may further comprise administering other therapeutic agents and/or therapies, e.g. cytokines such as interferons, TNF-α, IL-15, IL-2, or other anti-cancer drugs; radiation therapy; chemotherapy; monoclonal antibodies.

### EXAMPLES

### Materials and Methods

### 1.1 Reagents and Media Used in the Examples

| Reagent | Vendor | Cat. no. |
|---|---|---|
| RPMI1640 | Thermo Scientific | C11875500CP |
| PENICILLIN STREPTOMYCIN SOL | bioind | 03-031-1B |
| Fetal Bovine Serum | Gibco | 10099141C |
| Gentamicin | bioind | 03-035-1C |
| Amphotericin | bioind | 03-028-1B |
| HEPES | bioind | 03-025-1B |
| Sodium pyruvate (100 mM) | Invitrogen | 11360070 |
| MEM-NEAA (100X) | Lifetechnologies | 11140050 |
| β-Mercaptoethanol (55 mM) | Procell | PB180633 |
| L-Glutamine (200 mM) | Solarbio | G0200 |
| HBSS | Thermo Scientific | C14175500BT |
| Type IV Collagenase | ALDRICH | C5138-100MG |
| Type II Hyaluronidase | ALDRICH | H2126-100MG |
| DNase I Type IV | ALDRICH | D5025-15KU |
| ACK Red Blood Cell Lysis Buffer | Solarbio | R1010-500ml |
| Human IL-2 (rIL-2) | PeproTech | AF-200-02-10 |
| Human IL-7 (rIL-7) | PeproTech | AF-200-07-10 |
| Human IL-15 (rIL-15) | PeproTech | AF-200-15-10 |
| Anti-Human CD3 (OKT3) | Acro | CDE-M120a |
| Disodium hydrogenphosphate | MERYER | D54014-500G |
| dihydrate | | |
| Potassium Dihydrogen Phosphate | Solarbio | P7392 |
| Percoll | GE | 17-0891-02 |
| AIM V MED | Invitrogen | 12055083 |
| Human serum albumin | ACMEC | A93920-100mg |
| Collagenase from Clostridium | ALDRICH | C5138-100MG |
| histolyticum | | |
| IV DNase I | ALDRICH | D5025-15KU |
| Hyaluronidase from sheep testes | ALDRICH | H2126-100MG |
| Decitabine | Solarbio | D9010 |
| Recombinant Human IFN-γ/IFNG | Acrobiosystems | IFG-H4211-100ug |
| Recombinant Human TNF-α | Acrobiosystems | TNA-H4211-25ug |
| Human IFN-γ Valukine ELISA Kit | R&D Systems | VAL104 |
| Zombie NIR ™ Fixable Viability Kit | Biolegend | 423105 |
| PerCP-Cy5.5-CD45 | Biolegend | 304028 |
| Brilliant Violet 605 ™ anti-human | Biolegend | 301039 |
| CD8a (BV605-CD8) | | |
| Alexa Fluor 647 anti-human CD4 | Biolegend | 317422 |
| (APC-CD4) | | |
| FITC anti-human CD3 (FITC-CD3) | Biolegend | 300406 |
| Alexa Fluor 700 anti-human CD137 (4- | Biolegend | 309816 |
| 1BB) (AF700-CD137) | | |
| PE/Cyanine7 anti-human CD279 (PD- | Biolegend | 329917 |
| 1) (PE/Cy7-PD-1) | | |
| PE/Dazzle ™ 594 anti-human HLA- | Biolegend | 311439 |
| A, B, C | | |
| PE anti-human CD80 | Biolegend | 305207 |
| FITC anti-human CD86 | Biolegend | 374203 |
| FITC anti-human HLA-DR, DP, DQ | Biolegend | 361705 |
| PE anti-human CD252 (OX40L) | Biolegend | 326307 |
| PE/Dazzle ™ 594 anti-human CD274 | Biolegend | 329731 |
| (B7-H1, PD-L1) | | |
| FS Universal SYBR Green Master Rox | Roche | 4913850001 |
| 5 ml | | |
| Cryostor CS10 | STEMCELL | 07930 |
| | Technologies | |
| MTT | Beyotime | ST1537-5g |
| HiScript 1st Strand cDNA Synthesis | Vazyme Biotech | R111-01 |
| Kit | | |
| RNAprep pure Total RNA extraction | Tiangen | DP430 |
| kit | | |
| RNAiso Plus | Takara | 9108 |
| Human IFN-γ precoated ELISPOT kit | Dakewe | 2110005 |
| Alexa Fluor 700 Mouse IgG1, κ | Biolegend | 400143 |
| Isotype Ctrl (AF700-iso) | | |
| PE/Cyanine 7 Mouse IgG1, κ Isotype | Biolegend | 400125 |
| Ctrl (PE/Cy7-iso) | | |
| Human IL-12/IL-23p40 Valukine | R&D Systems | VAL121 |
| ELISA kit | | |

### 1.2 Media and Solutions Used in the Examples

### CCM Medium:

50 ml CCM medium was prepared as follows: RPMI 1640:42.5 ml; gentamicin solution: 500 μL; sodium pyruvate (100 mM): 500 μL; MEM-NEAA non-essential amino acid solution (100X): 500 μL; B-mercaptoethanol (55 mM): 13 μL; L-glutamine (200 mM): 500 μL; Gibco fetal bovine serum: 5 ml; HEPES: 500 μL; gentamicin: 10 μL; amphotericin B: 50 μL.

### REP Media I:

20 ml REP Media I was prepared: CCM: 20 ml; rIL-2 (10 μg/ml): 10 μL; rIL-7 (10 μg/ml): 20 μL; rIL-15 (10 μg/ml): 20 μL; OKT3 (500 μg/ml): 2 μL.

### REP Media II:

REP Media II was prepared by mixing REP Media I and AIM V medium at a 1:1 ratio.

### 199V Medium:

199V medium was prepared by adding 1% fetal bovine serum (BI) to Medium 199.

### Standard Isotonic Percoll (SIP) Solution:

SIP was freshly prepared by mixing Percoll with acidic PBS at a 19:1 ratio. 20X acidic PBS (pH 4.6, 1.051 g/ml) was prepared using: \textcircled{1} 6.75 g NaCl; \textcircled{2} 0.0625 g Na₂HPO₄·2H₂O; \textcircled{3} 1.05 g KH₂PO₄; \textcircled{4} 50 ml distilled water. To make 10 ml SIP, 500 μL acidic PBS and 9.5 ml Percoll were used. 60%, 45% and 34% SIP solutions for separation steps were prepared by diluting SIP with CCM.

### 1.3 Instruments and Equipment Used in the Examples

| Equipment/Instrument | Model |
|---|---|
| Analytical Flow Cytometer | BD LSRFortessa |
| Inverted Fluorescence Microscope | NIKON ECLIPSE Ts2 |
| Multimode Microplate Reader | SpectraMax i3x |
| Protein Purification System | Akta pure |
| Gel Documentation System | Tanon 1600 |
| Equipment/Instrument | Model |
| Real-Time Quantitative PCR (qPCR) | CFX touch 96 |
| System | |

### 1.4 the Nucleic Acid and Amino Acid Sequences Used in the Working Examples are Provided in the Sequence Listing.

### Example 1: Isolation, Expansion, and Identification of Tumor-Infiltrating Lymphocytes (TIL)

### Example 1.1: Isolation and Expansion of TIL

Primary oral cancer tissues were obtained from four patients (the first case (OC1): gingival cancer, the second case (OC2): buccal cancer, the third case (OC3): tongue cancer, the fourth case (OC4): gingival cancer, all four cases were squamous cell carcinoma). The tissues were placed in a petri dish, where they were minced into fragments smaller than 0.5 mm using a scalpel. The tissue fragments were then transferred to a 15 mL centrifuge tube, covered with 4 mL of digestion buffer, and incubated at 37° C. with shaking for 30 minutes. The digestion buffer contained: (1) 2 mg/mL type IV collagenase in Hank's Balanced Salt Solution (HBSS); (2) 10 mg/mL type II hyaluronidase in a solution containing 0.02 M phosphate-buffered saline (pH 7.2), 0.77 M NaCl, and 0.1% human serum albumin; (3) 5 mg/mL type IV DNase I in 0.15 M NaCl solution; and (4) 2.4 mL of CCM culture medium.

After digestion, the tumor pieces were crushed through a 70 μm strainer, and washed continuously with PBS until the final volume reached 20 mL. The suspension was then centrifuged at 400 g at room temperature for 3-5 minutes, the supernatant discarded, and the cells resuspended in 1 mL of ACK red blood cell lysis buffer. An additional 3 mL of ACK lysis buffer was added, and the tube was inverted to mix, then incubated at room temperature for 4 minutes. After adding 30 mL of PBS, the tube was centrifuged at 400 g for 3 minutes at room temperature, and the cells were resuspended in 20 mL PBS and filtered through a 70 μm mesh. The cells were then evenly distributed into a 24-well plate, with 250,000 cells in 500 μL per well. After overnight incubation, the supernatant was carefully collected and centrifuged at 400 g for 5 minutes at room temperature, and the cells were resuspended in 3 mL of 60% SIP and transferred to a 15 mL centrifuge tube. Slowly, 3 mL of 45% SIP were layered on top of the 60% SIP, and then 3 mL of 34% SIP were gently added on top of the 45% SIP. The tube was then centrifuged at 2400 g at room temperature for 30 minutes. The interface between 60% and 45% SIP (2-3 mL) was collected into a 15 mL centrifuge tube and topped up with PBS. After centrifugation at 600 g for 10 minutes at room temperature, the cell pellet was resuspended in 500 μL of REP Media I containing low-dose IL-2 (100 IU/ml), IL-7, and IL-15, and incubated at 37° C. with 5% CO2, subculturing to maintain a density of 1×10\hat{ }6 cells/ml. On day 5 post-subculture, 70% of the REP Media I was replaced with REP Media II. The cells were cultured in a 24-well plate for 10-22 days until the cell count reached about 5×10\hat{ }7, at which point a rapid expansion was performed.

Rapid Expansion and Reinfusion Steps: On day 1, the 4×10\hat{ }5 TIL cells obtained from the aforementioned isolation and culture steps were resuspended in 50 mL of REP Media I and placed in a vertically oriented T75 culture flask. On day 5, 65% of the culture medium was replaced with REP Media II containing 3000 IU/mL IL-2. Starting from day 5, the total number of viable cells was measured every other day. When the number of the cells reached 1.4×10\hat{ }7, the TIL cells were resuspended in 100 μL of 0.9% saline and injected intratumorally into an animal model within 30 minutes.

### Example 1.2 Characterization of Anti-tumor Properties of TIL Cells

The anti-tumor properties of activated OC1-TIL cells were assessed through tumor cell cytotoxicity assays and ELISA assays, which demonstrated their ability to specifically kill tumor cells.

### Tumor Cell Cytotoxicity Assay

Several studies have shown that a DEC cocktail containing decitabine (a DNA methylation inhibitor), TNF-α, and IFN-γ can restore the expression of various antigens on the surface of tumor cells. To demonstrate that TILs can target and destroy autologous oral cancer cells, primary oral cancer cells (OC1-TC) treated with the DEC cocktail were cultured with autologous TILs (OC1-TIL), and the cytotoxic effect of OC1-TIL on primary oral cancer cells was detected.

In brief, 5,000 primary oral cancer cells (OC1-TC) were plated in each well of a 96-well plate, and after 24 hours, the supernatant was replaced with a DEC cocktail culture medium (10 μM DEC, 100 U/mL IFN-γ, and 10 ng/mL TNF-α) for an additional 48 hours. Then, the supernatant was discarded, and TILs were added at various effector-to-target (E:T) ratios (OC1-TC alone, TIL:OC1-TC at 1:1, 5:1, or 10:1). The cells were resuspended in 100 μl REP media I, and cultured for another 24 hours before the supernatant was collected. The 96-well plate was carefully washed three times with PBS, and the results were observed under a microscope and photographed.

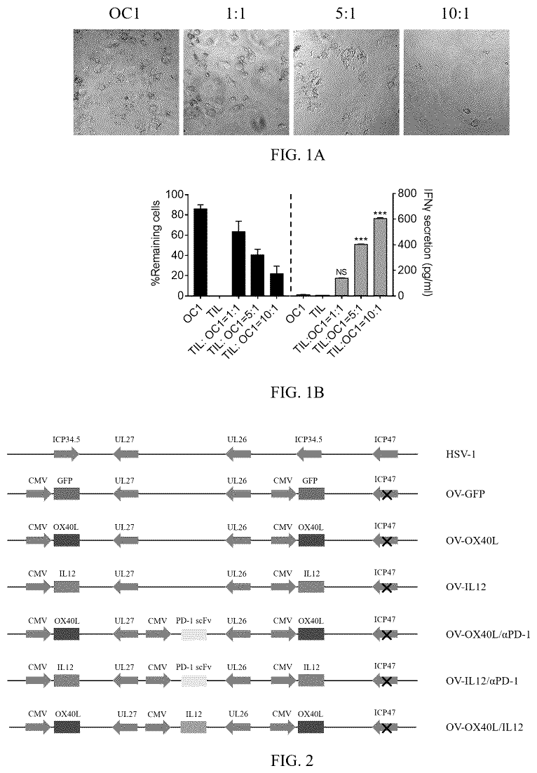

As shown in FIG. 1A, the co-culture of OC1 and TILs significantly reduced the number of primary oral cancer cells, and the reduction was proportional to the E:T ratio.

### IFN-γ ELISA Detection

Previous research has indicated that the activation level of TILs is directly proportional to the release of IFN-γ. To further demonstrate that OC1-TC treated with the DEC cocktail can activate TILs, OC1-TC cells were cultured in a 96-well plate for 48 hours as described above, followed by the addition of TILs at various E:T ratios for another 24 hours. ELISA was used to measure the content of IFN-γ in the supernatant of each group (OC1-TC alone, TIL alone, and TIL: OC1-TC co-cultures at 1:1, 5:1, or 10:1), and the viable OC1-TC cells was counted.

The Human IFN-γ Valukine ELISA Kit (1 KT) (R&D Systems, VAL104) was used according to the manufacturer's instructions to measure the content of IFN-γ in the supernatant. The wells of the microplate were loaded with various concentrations of standard solutions or experimental samples, 100 μl per well. The reaction wells were sealed with sealing tape and incubated at room temperature for 2 hours. Each well was washed with 400 μl of wash buffer, and the wash was repeated four times. Then, 200 μl of the enzyme-linked detection antibody was added to each micro-well and incubated at room temperature for 2 hours; after washing, 200 μl of substrate solution was added to each micro-well and incubated at room temperature in the dark for 30 minutes; then, 50 μl of stop solution 1 was added to each well, changing the color of the solution from blue to yellow. Within 30 minutes of adding stop solution 1, the absorbance at 450 nm was measured using a microplate reader, with 540 nm or 570 nm set as the reference wavelength.

The experimental results (FIG. 1B) showed that OC1-TC treated with the DEC cocktail activated TILs, with The level of activation correlated with the E:T ratio. When the E:T ratio reached 10, more than 80% of OC1-TC cells were eliminated, and about 600 μg/ml of IFN-γ was released from OC1-TILs.

These results demonstrate that when the ratio of TILs to OC1-TC reaches a certain level, the activated TILs specifically recognize and destroy autologous tumors, exhibiting the specificity for the tumor.

### Example 2: Engineering and Characterization of Oncolytic Viruses

### Example 2.1 Engineering of Oncolytic Viruses (OVs)

The wild-type OV (HSV-1) was isolated from patients with oral herpes infections. Construction of the viruses in this study were performed as follows: OV-GFP was obtained by replacing the ICP34.5 gene in the HSV-1 genome with a GFP expression cassette and deleting the ICP47 gene from the genome. Based on OV-GFP, the GFP expression cassette was replaced with a cell membrane-displayed trimeric OX40L (the extracellular domain of OX40L fused to the trimerization domain of TRAF2, further fused via a flexible linker to the transmembrane domain for expression; SEQ ID NOs: 3 and 18) or with an IL-12 gene (SEQ ID NOs: 1-2 and 16-17), resulting in OV-OX40L and OV-IL-12, respectively. On the basis of OV-OX40L and OV-IL-12, the PD-1 scFv gene (SEQ ID NOs: 4-5 and 19) was inserted into the intergenic region between the UL26 and UL27, resulting in OV-OX40L/αPD-1 and OV-IL-12/αPD-1. Furthermore, based on OV-OX40L, the PD-1 scFv gene was inserted into the intergenic region between the UL26 and UL27, and the IL-12 sequence was inserted into the intergenic region between the UL3 and UL4, resulting in OV-OX40L/IL-12/αPD-1; or based on OV-OX40L, the IL-12a-IRES2-IL-12b-T2A-PD-1 scFv gene was inserted into the intergenic region between the UL26 and UL27, resulting in OV-OX40L/IL-12/αPD-1. The schematic diagram of the oncolytic virus modification is shown in FIG. 2. Deletion of ICP34.5 from HSV-1 enhances selective replication of virus in tumors, and deletion of ICP47 increases antigen presentation and improves oncolytic activity.

### OV-GFP Virus Construction

In order to construct the pICP34.5-HA2L-HA2R plasmid, homology arms HomologyArm2L (HA2L) and HomologyArm2R (HA2R) flanking the ICP34.5 coding region were amplified by PCR from HSV-1 DNA (sequences are provided in the supplementary table). HA2L and HA2R were cloned flanking the CMV-GFP-SV40polyA to serve as donor DNA. The donor DNA was transfected into 293 FT cells for 24 hours, followed by infection with HSV-1 for 48 hours. After three rounds of plaque screening, the OV-GFP precursor virus was obtained.

Following the same method, the pICP47-HA3L-HA3R plasmid was constructed, in which the homology arms HA3L and HA3R (sequences are provided in the supplementary table) flanking the ICP47 coding region were cloned on either side of CMV-RFP-SV40polyA as donor DNA. This donor DNA was transfected into 293 FT cells for 24 hours, and then the cells were infected with the OV-GFP precursor virus for 48 hours. After three rounds of plaque screening, the OV-GFP virus was obtained.

Construction of OV-OX40L/GFP Virus with GFP Expression Cassette Inserted Between the UL26-UL27 Genes

To insert the PD-1 scFv gene into the intergenic region between the UL26-UL27 genes of the virus, the GFP expression cassette CMV-GFP-SV40polyA was recombinantly constructed into the UL26-UL27 intergenic region of OV-OX40L through homologous recombination of the viral genome with a donor plasmid. The donor plasmid used for this process included a 1471 bp left homology arm, a GFP expression cassette, and a 1339 bp right homology arm (specific sequence information for the left and right homology arms HALL and HAIR is provided in the supplementary table).

Construction of OV-OX40L/αPD-1/GFP Virus with GFP Expression Cassette Inserted Between the UL3-UL4 Genes

To insert the IL-12 gene into the intergenic region between the UL3-UL4 genes of the virus, the GFP expression cassette CMV-GFP-SV40polyA was recombinantly constructed into the UL3-UL4 region of OV-OX40L/αPD-1 through homologous recombination of the viral genome with a donor plasmid. The donor plasmid used for this process included an 1113 bp left homology arm, a GFP expression cassette, and a 1031 bp right homology arm (specific sequence information for the left and right homology arms HA4L and HA4R is provided in the supplementary table).

### Construction of Plasmids for Target Gene Fragments

The IL-12 target gene fragments were ordered from Beijing Yiqiao Shenzhou Biotechnology Co., Ltd. (IL-12 p35/IL-12A cDNA ORF Clone in Cloning Vector, Human: HG10021-M; and IL-12B cDNA ORF Clone in Cloning Vector, Human: HG10052-M; OX40L: HG13127); and the PD-1 scFv sequence was obtained from the Drugbank database. PCR was used to add appropriate restriction sites to the ends of the target sequences. The plasmids containing the target gene fragments and the pCMV-eGFP plasmid (UboBio) containing homology arms (with CMV promoter and SV40 polyA signal serving as homology arms) were double-digested respectively, and ligated. After transformation and plating, single colonies were picked and sequenced for verification. This resulted in the plasmids having the target fragments inserted, namely pCMV-OX40L, pCMV-IL-12, and pCMV-PD-1_scFv.

### Armed Oncolytic Virus Production:

6×10⁵ 293 FT-A5 cells were plated in a six-well plate and cultured for 12-18 h until about 70%-80% confluence was reached. The cells were transfected with 4 μg of pCMV-OX40L or pCMV-IL-12 plasmid using 10 μl of PEI transfection reagent. After transfection at 37° C. for 6 h, the medium was replaced with a fresh culture medium. 24 h post-transfection, the cells were infected with 1×10⁵ PFU of OV-GFP virus. After 2 h, the medium was replaced with 199V medium containing no virus. Cells were cultured for another 48 hours. Then, the cells and the medium were collected into sterile centrifuge tubes, and centrifuged at 4° C., 3000 rpm for 10 min. The supernatant was discarded, and the pellet was resuspended in 100 μl of 9% sterile skim milk, subjected to three freeze-thaw cycles between −80° C. and room temperature, and aliquoted into 500 μL tubes. Gradient concentration screening was performed in Vero cells, picking viral plaques for a second-round screening. After three rounds of selection, viral DNAs were extracted using a viral DNA extraction kit and PCR were performed to check if the expression cassette having the CMV promoter-(OX40L or IL-12)-SV40 polyA signal was inserted into the viral genome. The oncolytic viruses OV-OX40L and OV-IL-12 were obtained.

Using the same transfection-infection method as above, and using the plasmid pCMV-PD-1_scFv, the expression cassette containing the CMV promoter-PD-1 scFv gene-SV40 polyA signal was inserted at the UL26UL27 site of OV-OX40L and OV-IL-12 through homologous recombination, yielding OV-OX40L/αPD-1 and OV-IL-12/αPD-1. In both OV-OX40L/αPD-1 and OV-IL-12/αPD-1, the expression of OX40L or IL-12 and the expression of the PD-1 scFv gene were not interfered with each other due to their different insertion sites and independent expression systems.

Based on OV-OX40L, the expression cassette containing the CMV promoter-hIL-12-polyA signal sequence was inserted between UL26 and UL27, obtaining the two-factor armed oncolytic virus OV-OX40L/IL-12.

### Characterization of Armed Oncolytic Virus

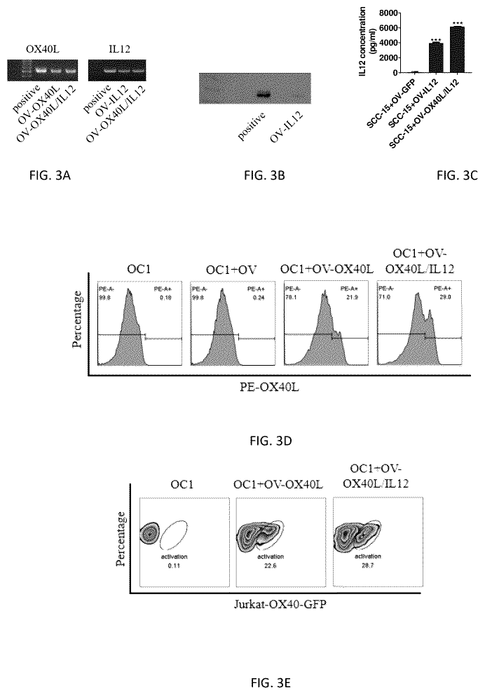

Firstly, the insertions of the genes of interest were verified at the genetic level. The viral genomes of OV-OX40L, OV-IL-12, OV-OX40L/αPD-1, and OV-IL-12/αPD-1 were extracted using a kit, and then the genomes were amplified with the corresponding primers. The results are shown in FIG. 3A.

Secondly, after viral infection of primary oral cancer cells, the expression and activity of OX40L and IL-12 were assessed at the protein level. To detect IL-12 expression, the Western blot experiment shown in FIG. 3B were conducted. Oral cancer cells were infected with OV-IL-12 (MOI=0.01) for 48 hours. The culture supernatant was collected after 48 hours, denatured, and subjected to electrophoresis. Then the gels were scanned and photographed with a gel imaging system, where an IL-12 protein positive control was included for comparison. Additionally, in the ELISA experiment shown in FIG. 3C, oral cancer cells were infected with OV-GFP (MOI=0.01), OV-IL-12 (MOI=0.01), and OV-OX40L/IL-12 (MOI=0.01) for 48 hours. The culture supernatant was collected after the 48-hour infection, and the Human IL-12/IL-23p40 Valukine ELISA kit was used according to the manufacturer's instructions to detect IL-12 concentration in the supernatant. As shown in FIGS. 3B and 3C, Western Blot and ELISA confirmed the expression of IL-12 in oral cancer cells infected with OV-IL-12 and OV-OX40L/IL-12.

To assess the expression and activity of OX40L, oral cancer cells were infected with wild type OV (MOI=0.01), OV-OX40L (MOI=0.01), and OV-OX40L/IL-12 (MOI=0.01) for 48 hours, as shown in the Flow Cytometry assay in FIG. 3D. After the 48 hours infection, cells from each group were collected for staining, following the procedures outlined in Example 1.2. Additionally, in the Reporter Cell Assay depicted in FIG. 3E, oral cancer cells were infected with OV-OX40L (MOI=0.01) and OV-OX40L/IL-12 (MOI=0.01) for 48 hours, then cultured with Jurkat-OX40-GFP cells for 24 hours. After the culturation, the suspended cells were collected to measure the proportion of GFP-positive cells using flow cytometry. As demonstrated in FIGS. 3D and 3E, flow cytometry staining and reporter cell assays confirmed that the trimeric OX40L expressed on the surface of oral cancer cells can bind and activate OX40.

### Example 2.2 Characterization of the Oncolytic Properties of the engineered Armed Oncolytic Virus

Evaluation of Cytolytic Effects of Armed Oncolytic Virus at Various Titers on Primary Oral Cancer Cells from Multiple Patients Using MTT Assay

To evaluate the ability of oncolytic viruses to infect oral cancer, we infected primary oral cancer cells and patient-derived oral cancer tissues with OV-GFP and engineered armed oncolytic viruses at various multiplicities of infection (MOI). Specifically:

The primary oral cancer cells OC1, OC2, OC3, and OC4 were infected with oncolytic viruses OV-GFP, OV-OX40L, OV-IL-12, and OV-OX40L/IL-12 respectively, establishing the following experiment groups:

$$OC 1 \text{/} 2 \text{/} 3 \text{/} 4 + OV \text{-} GFP ( MOI : 0 , 0.01 , 0.1 , 1 , 10 , 100 )$$

$$OC 1 \text{/} 2 \text{/} 3 \text{/} 4 + OV \text{-} OX 40 L ( MOI : 0 , 0.01 , 0.1 , 1 , 10 , 100 )$$

$$OC 1 \text{/} 2 \text{/} 3 \text{/} 4 + OV \text{-} IL \text{-} 12 ( MOI : 0 , 0.01 , 0.1 , 1 , 10 , 100 )$$

$$OC 1 \text{/} 2 \text{/} 3 \text{/} 4 + OV \text{-} OV \text{-} OX 40 L \text{/} IL \text{-} 12 ( MOI : 0 , 0.01 , 0.1 , 1 , 10 , 100 ) .$$

After incubating the primary oral cancer cells with the oncolytic viruses for a period, the cytolytic effect of the viruses was measured using the MTT assay. Briefly, in a 96-well plate, each well was seeded with 5,000 primary oral cancer cells. After 24 hours, the supernatant was discarded, and 100 μl of a prepared virus dilution solution (virus serially diluted in CCM medium) was added to each well. The plate was centrifuged at 2000 rpm for 10 minutes and then incubated for an additional 2 hours. Following this incubation, the supernatant in each well was replaced with 100 μl of fresh CCM medium, and the plate was incubated for another 48 hours. After this period, the plates were centrifuged at 3000 rpm for 5 minutes, the supernatant was carefully aspirated, and 80 μl of fresh RPMI 1640 medium was added, followed by 20 μl of MTT solution (5 mg/ml, i.e., 0.5% MTT). The plate was then incubated in the dark for 4 hours. After incubation, the supernatant was aspirated by centrifugation at 3000 rpm for 5 minutes, and 150 μl of dimethyl sulfoxide (DMSO) was added to each well. The plate was shaken for 2 minutes and incubated for 30 minutes to fully dissolve the formazan crystals. A blank well was set up for zero calibration, using the PBS group (MOI=0) where DMSO was added after MTT incubation. The absorbance of each well was measured at 490 nm using an enzyme-linked immunosorbent assay reader. Relative cell viability was determined in relation to the absorbance of the blank well.

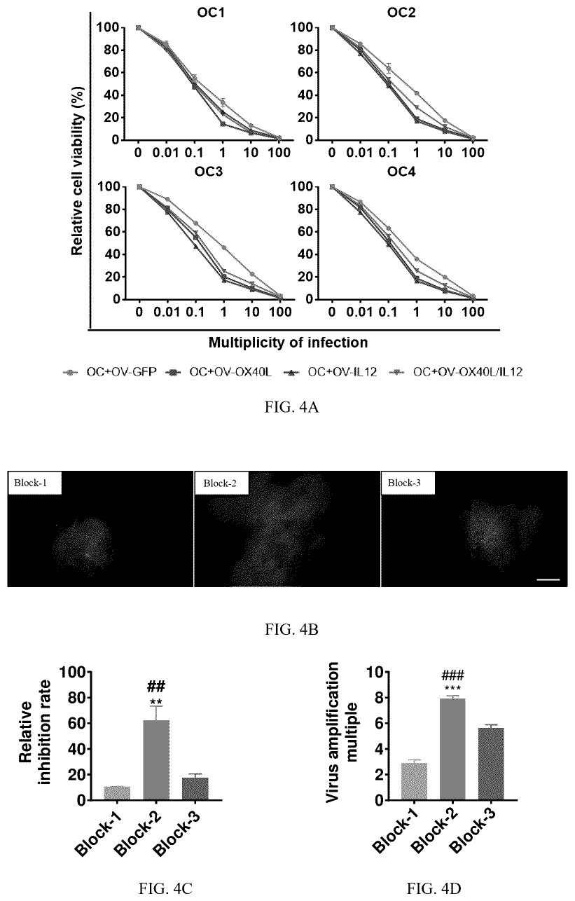

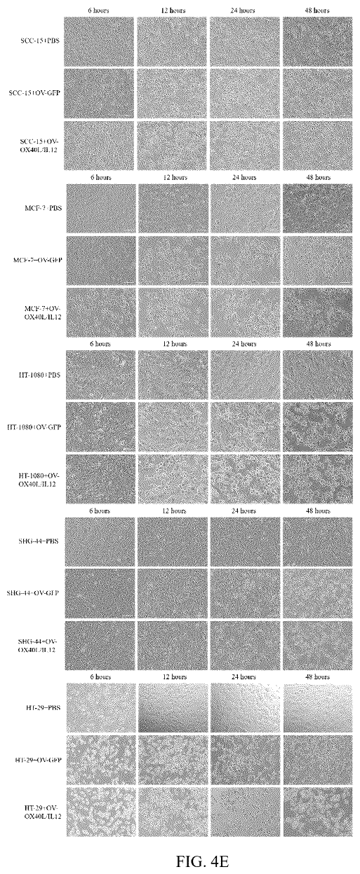

As shown in FIG. 4A, the oncolytic viruses effectively infected and lysed oral cancer cells, with no significant difference in cytolytic ability on oral cancer cells observed between the engineered oncolytic viruses and the parental oncolytic virus.

### Assessment of the Cytolytic Effect of the Oncolytic Virus on Patient-Derived Primary Oral Cancer Tissues

In the following experiments, the parental oncolytic virus OV-GFP or the corresponding wild-type oncolytic virus OV without GFP was used to assess the cytolytic effect of the oncolytic virus alone on primary cancer tissues.

### Fluorescence Microscopy:

(1) Prepared a 96-well plate and added 100 μl of CCM medium to wells A1-A4;
(2) Placed the primary oral cancer tissue (OC1) in a sterile culture dish. Punctured the tumor with a 2 mm tumor sampler, then used a scalpel to cut the punctured sample into four parts, and weighed them with an analytical balance;
(3) Used a pipette tip to transfer the tissue blocks into wells A1-A4 of the 96-well plate;
(4) Added 1×10⁵ PFU of OV-GFP to wells A1-A3, with well A4 serving as the blank control. After incubating at 37° C. for 48 hours, observed under a fluorescence microscope.

The observation results are shown in FIG. 4B. In the figure, blocks 1-3 correspond to wells A1-A3 respectively.

### Virus Titration Assay

To detect the viral titers in the supernatant of tumor tissue samples infected with OV-GFP, the supernatant was collected after the 48-hour incubation in the aforementioned step (4) and serially diluted. The virus serial dilutions were added at 800 μl/well to a 6-well plate pre-seeded with 5×10⁵ Vero cells. After incubating for 2 hours, the medium was replaced with fresh 199V medium for a further 36-hour incubation. Thereafter, virus plaques per well were counted under a microscope, and the virus amplification multiple was calculated. The results are shown in FIG. 4C.

### Assay for Relative Inhibition Rate

In this assay, to avoid interference from GFP fluorescence in the Alamar Blue assay, the wild-type OV corresponding to OV-GFP was used to assess the cytotoxic effects of OV on primary oral cancer tissue. Briefly:
1. Prepared a 24-well plate and added 2 ml of medium to wells A1-A4.
2. Placed the primary oral cancer tissue (OC1) in a sterile culture dish, punctured the tumor with a 2 mm tumor sampler, cut the sample into four equal parts with a scalpel, and weighed them using an analytical balance.
3. Transferred the tumor tissues to wells A1-A4 respectively using a pipette tip.
4. Added 25 μl/well of Alamar Blue cell viability reagent (Beyotime) and incubated for 1 hour at 37° C.
5. After incubation, transferred 300 μl from each well to three parallel wells of the 96-well plate.
6. Measured the fluorescence using a plate reader (excitation: 530 nm; emission: 590 nm) and recorded the data.
7. After recording the data, transferred the tissue blocks from row A to row B and added fresh medium.
8. Wells B1-B3 corresponded to blocks 1, 2, and 3, respectively. Added 25 μl of wild-type OV (1×10⁶ PFU/ml) to each of wells B1-B3; well B4 remained without wile type OV addition. After 72 hours, added 25 μl of Alamar Blue and repeated steps 4 to 6. Calculated the relative inhibition rate. The results are shown in FIG. 4D.

### Results:

As shown in FIG. 4B, OV-GFP successfully infected and invaded the OC1 primary oral cancer tissue. FIG. 4C illustrates the viral titers measured in the supernatants from the three wells corresponding to blocks 1-3. The results indicate that OV-GFP amplified 2-8 times within 36 hours in the oral cancer tissue, demonstrating its ability to replicate in primary oral cancer tissue. FIG. 4D assesses the effect of OV on cell viability of oral cancer tissue using the Alamar Blue assay, showing that at 72 hours, OV achieved a 60% inhibition rate in the block-2 sample, confirming that OV-GFP has cytotoxic effects on oral cancer tissue. Overall, FIG. 4 demonstrates that OV-GFP can infect, replicate, and kill cancer cells across different regions of primary cancer tissue, while also exhibiting tissue regional heterogeneity.

### Cytotoxicity of Oncolytic Virus OV-OX40L/IL-12 in Various Tumor Cell Lines

To demonstrate that OV-OX40L/IL-12 can infect various tumor cell types, we infected five cell lines with the oncolytic virus: SCC-15 (human oral squamous cell carcinoma cell line, ATCC CRL-1623), SHG-44 (human glioma cell line), MCF-7 (human breast cancer epithelial cell line, ATCC HTB-22), HT-29 (human colon cancer cell line, ATCC HTB-38), and HT-1080 (human fibrosarcoma cell line, ATCC CCL-121). The experimental groups included PBS, OV-GFP (MOI=0.1), and OV-OX40L/IL-12 (MOI=0.1). At 6, 12, 24, and 48 hours post oncolytic virus infection, the cells in wells of each group were observed and photographed under a microscope. The results are presented in FIG. 4E.

Results: The oncolytic virus OV-OX40L/IL-12 effectively infected various tumor cell lines, including human oral squamous cell carcinoma, glioma, breast cancer, colon cancer, and fibrosarcoma.

In conclusion, these data indicate that OV-OX40L, OV-IL-12, OV-OX40L/αPD-1, and OV-IL-12/αPD-1 can infect and kill primary oral cancer cells and tissues, and are capable of expressing OX40L, IL-12, and PD-1 scFv.

### Example 3: Effect of Oncolytic Virus Combined with TIL in Oral Cancer

### Example 3.1 In Vitro Combination of Oncolytic Virus and TIL

Co-Culture Experiments for Detection of the Killing Effect of OV-OX40L/αPD-1 and OV-IL-12/αPD-1 Combined with TIL on Primary Oral Cancer Cells.

Primary oral cancer cells (OC1-TC) were pre-infected with armed oncolytic viruses at MOI=0.01 for 48 hours, followed by the addition or non-addition of TIL and incubation for another 24 hours. Cells were observed and photographed under a microscope. As controls, OC1 primary cells without addition of oncolytic virus and TIL, and OC1 primary cells with only TIL added, were examined. Briefly, the following groups were set up in the experiment:

$$OC 1 cells only \\ OC 1 + OV \text{-} GVP ( OC 1 cells infected with oncolytic virus OV \text{-} GFP ) \\ OC 1 + OV \text{-} OX 40 L ( OC 1 cells infected with oncolytic virus OV \text{-} OX 40 L ) \\ OC 1 + OV \text{-} IL \text{-} 12 ( OC 1 cells infected with oncolytic virus OV \text{-} IL \text{-} 12 ) \\ OC 1 + OV \text{-} OX 40 L \text{/} IL \text{-} 12 ( OC 1 cells infected with oncolytic virus OV \text{-} OX 40 L \text{/} IL \text{-} 12 )$$

$$OC 1 + OV \text{-} OX 40 L \text{/} α PD \text{-} 1 ( OC 1 cells infected with oncolytic virus OV \text{-} OX 40 L \text{/} α PD \text{-} 1 )$$

$$OC 1 + OV \text{-} OX 40 L \text{/} α PD \text{-} 1 ( OC 1 cells infected with oncolytic virus OV \text{-} IL \text{-} 12 \text{/} α PD \text{-} 1 )$$

$$OC 1 + OV \text{-} OX 40 L \text{/} IL \text{-} 12 \text{/} α PD \text{-} 1 ( OC 1 cells infected with oncolytic viruses OV \text{-} OX 40 L \text{/} α PD \text{-} 1 + OV \text{-} IL \text{-} 12 \text{/} α PD \text{-} 1 at 1 : 1 ratio )$$

$$OC 1 + TIL ( OC 1 cells co \text{-} cultured with TIL ) \\ OC 1 + OV \text{-} GFP + TIL ( OC 1 cells infected with OV \text{-} GFP co \text{-} cultured with TIL ) \\ OC 1 + OV \text{-} OX 40 L + TIL ( OC 1 cells infected with OV \text{-} OX 40 L co \text{-} cultured with TIL ) \\ OC 1 + OV \text{-} IL \text{-} 12 + TIL ( OC 1 cells infected with OV \text{-} IL \text{-} 12 co \text{-} cultured with TIL ) \\ OC 1 + OV \text{-} OX 40 L \text{/} IL \text{-} 12 + TIL ( OC 1 cells infected with OV \text{-} OX 40 L \text{/} IL \text{-} 12 co \text{-} cultured with TIL )$$

$$OC 1 + OV \text{-} OX 40 L \text{/} α PD \text{-} 1 + TIL ( OC 1 cells infected with OV \text{-} OX 40 L \text{/} α PD \text{-} 1 co \text{-} cultured with TIL )$$

$$OC 1 + OV \text{-} IL \text{-} 12 \text{/} α PD \text{-} 1 + TIL ( OC 1 cells infected with OV \text{-} IL \text{-} 12 \text{/} α PD \text{-} 1 co \text{-} cultured with TIL )$$

$$OC 1 + OV \text{-} OX 40 L \text{/} IL \text{-} 12 \text{/} α PD \text{-} 1 + TIL ( OC 1 cells infected with OV \text{-} OX 40 L \text{/} α PD \text{-} 1 + OV \text{-} IL \text{-} 12 \text{/} α PD \text{-} 1 at 1 : 1 ratio co \text{-} cultured with TIL )$$

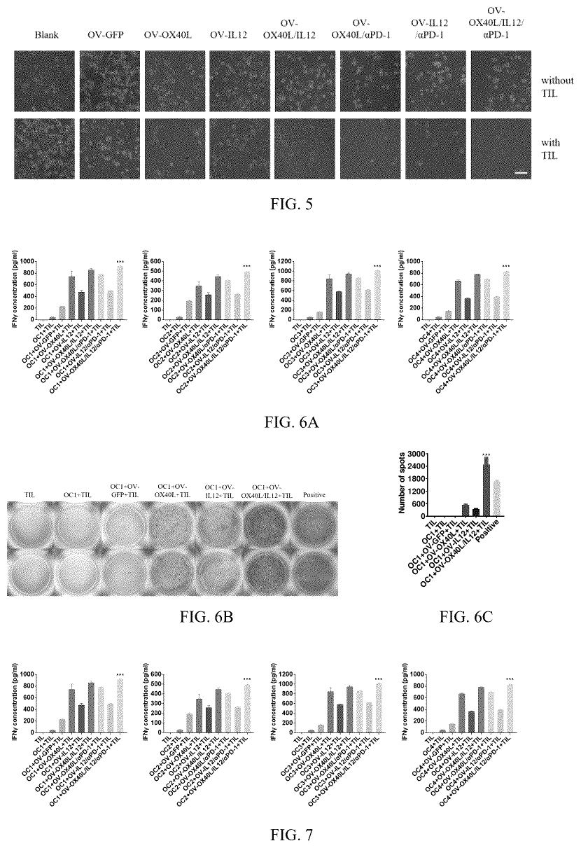

In a 96-well plate, 5000 primary oral cancer cells were seeded per well in duplicates for each group. After 24 h, armed oncolytic viruses were infected at MOI=0.01 and incubated for another 48 h. Where the infection was performed by using the combination of different oncolytic virus, the ratio of the viruses was 1:1. After the 48 h incubation, the supernatant was discarded, and TILs were added at an E:T ratio of 1:1. The cells were resuspended in 100 μl REP media I, and co-cultured for 24 h before collecting the supernatant. The 96-well plate was carefully washed 3 times with PBS, observed and photographed under a microscope. The results are shown in FIG. 5.

ELISA Assay for Detection of the Activation of TILs by Primary Oral Cancer Cells Pre-Infected with OV-OX40L/αPD-1 and OV-IL-12/αPD-1

The following groups were set up, and the concentration of IFN-γ in the cell culture supernatants from each group was measured by ELISA to reflect the activation of TILs by the pre-infected oral cancer cells:
TILs only
Co-cultures of primary oral cancer cells with TILs: OC1/2/3/4+TIL
Co-cultures of primary oral cancer cells pre-infected with different oncolytic viruses (OV-GFP, OV-OX40L, OV-IL-12, OV-OX40L/IL-12, OV-OX40L/αPD-1, OV-IL-12/αPD-1 or OV-OX40L/αPD-1+OV-IL-12/αPD-1 (i.e. OV-OX40L/IL-12/αPD-1)) with TILs: OC1/2/3/4+OV-GFP+TIL, OC1/2/3/4+OV-OX40L+TIL, OC1/2/3/4+OV-IL-12+TIL, OC1/2/3/4+OV-OX40L/IL-12+TIL, OC1/2/3/4+OV-OX40L/αPD-1+TIL, OC1/2/3/4+OV-IL-12/αPD-1+TIL, OC1/2/3/4+OV-OX40L/IL-12/αPD-1+TIL.

### Experimental Steps:

1. Four 96-well plates were prepared, and 5,000 primary oral cancer cells were seeded per well. After 24 hours, the supernatant was discarded, and 100 μl of prepared virus dilution (MOI=0.01) was added. For the combination of two oncolytic viruses, the infection ratio was 1:1. The plates were centrifuged at 2,000 rpm for 10 minutes and then placed in the incubator for an additional 2 hours. After the incubation, the supernatant was replaced with 100 μl of fresh CCM medium per well, and the cells were cultured for another 48 hours.
2. Tumor-specific TILs were added at an E:T ratio of 1:1. To do this, after counting the tumor cells, the TILs were diluted to 2×10⁵ cells/ml, and 100 μl of the cell suspension (resuspended in CCM) was added to each well. The culture was continued for 24 hours.
3. After 24 hours, the supernatant was collected, centrifuged at 2,000 rpm for 5 minutes, and transferred to clean EP tubes for testing.
4. The samples were diluted 5-fold and measured using the Human IFN-γ Valukine ELISA kit (R&D Systems). The results are shown in FIG. 6A.
   ELISPOT Assay for Detection of the Activation of Tumor-Specific TILs by Oral Cancer Cells Pre-Infected with OV-OX40L, OV-IL-12 and OV-OX40L/IL-12

In this experiment, the multiplicity of virus infection was 0.01, and the following groups were set up:
TILs only
Co-culture of uninfected primary oral cancer cells with TILs: OC1+TIL
Co-culture of OV-GFP-infected primary oral cancer cells with TILs: OC1+OV-GFP+TIL
Co-culture of OV-OX40L-infected primary oral cancer cells with TILs: OC1+OV-OX40L+TIL
Co-culture of OV-IL-12-infected primary oral cancer cells with TILs: OC1+OV-IL-12 +TIL
Co-culture of OV-OX40L/IL-12-infected primary oral cancer cells with TILs: OC1+OV-OX40L/IL-12+TIL
Positive control: TILs treated with 2.5 μg/ml PHA.

### Experimental Steps:

1. 2×10⁵ OC1 cells were seeded in a well of a 24-well plate. After 24 hours, the cells were treated with a DEC cocktail for 48 hours, after which the supernatant was discarded. 2×10⁶ TILs, resuspended in 1 ml of REP media I, were added to the well for co-culture for one week. Subsequently, the TIL cells were cultured overnight in medium without cytokines and then were re-stimulated with DEC cocktail-treated OC1 cells for 6 hours.
2. OC1 cells were seeded in a 96-well plate. Of the seven total groups, five had OC1 cells pre-seeded.
3. Two replicates were set up for each group, resulting in a total of 15 wells. 1×10⁵ OC1 cells were resuspended in 2 ml of CCM medium, and 100 μl was added to each well.
4. After 24 hours, engineered oncolytic viruses (MOI=0.01) were added to the respective wells for a further 48-hour incubation.
5. After the 48-hour incubation, the stimulated TILs (5×10⁴ per well, and resuspended in REP media I) were added.
6. After 24 hours of co-culture, the TILs were collected from the supernatants of each group, washed three times with PBS, resuspended in 100 μl of CCM medium, and transferred to the pre-coated ELISPOT plate (Cat #2110005).
7. The manufacturer's instructions were followed for the staining steps.

The results are shown in FIGS. 6B and 6C.

MTT Assay for Detection of the Killing Effect of OV-OX40L/αPD-1 and OV-IL-12/αPD-1 Combined with TILs on Primary Oral Cancer Cells.

In this experiment, the virus titer was MOI=0.01. The following groups were set up:
Uninfected primary oral cancer cells without TILs: OC1/2/3/4+PBS;
Primary Oral cancer cells pre-infected with different oncolytic viruses (OV-GFP, OV-OX40L, OV-IL-12, OV-OX40L/IL-12, OV-OX40L/αPD-1, OV-IL-12/αPD-1 or OV-OX40L/αPD-1+OV-IL-12/αPD-1 (i.e. OV-OX40L/IL-12/αPD-1)):
   OC1/2/3/4+OV-GFP, OC1/2/3/4+OV-OX40L, OC1/2/3/4+OV-IL-12, OC1/2/3/4+OV-OX40L/IL-12, OC1/2/3/4+OV-OX40L/αPD-1, OC1/2/3/4+OV-IL-12/αPD-1, OC1/2/3/4+OV-OX40L/IL-12/αPD-1;
Co-cultures of primary oral cancer cells with TILs: OC1/2/3/4+TIL
Co-cultures of primary oral cancer cells pre-infected with different oncolytic viruses (OV-GFP, OV-OX40L, OV-IL-12, OV-OX40L/IL-12, OV-OX40L/αPD-1, OV-IL-12/αPD-1 or OV-OX40L/IL-12/αPD-1) with TILs:

$$OC 1 \text{/} 2 \text{/} 3 \text{/} 4 + OV \text{-} GFP + TIL , \\ OC 1 \text{/} 2 \text{/} 3 \text{/} 4 + OV \text{-} OX 40 L + TIL , \\ OC 1 \text{/} 2 \text{/} 3 \text{/} 4 + OV \text{-} IL \text{-} 12 + TIL , \\ OC 1 \text{/} 2 \text{/} 3 \text{/} 4 + OV \text{-} OX 40 L \text{/} IL \text{-} 12 + TIL , \\ OC 1 \text{/} 2 \text{/} 3 \text{/} 4 + OV \text{-} OX 40 L \text{/} α PD \text{-} 1 + TIL , \\ OC 1 \text{/} 2 \text{/} 3 \text{/} 4 + OV \text{-} IL \text{-} 12 \text{/} α PD \text{-} 1 + TIL , \\ OC 1 \text{/} 2 \text{/} 3 \text{/} 4 + OV \text{-} OX 40 L \text{/} IL \text{-} 12 \text{/} α PD \text{-} 1 + TIL .$$

### Experimental Steps:

1. Four 96-well plates were prepared, and 5,000 primary oral cancer cells were seeded per well. After 24 hours, the supernatant was discarded, and 100 μl of the prepared virus dilution (MOI=0.01) was added. The plates were centrifuged at 2,000 rpm for 10 minutes and then placed in the incubator for another 2 hours. After this incubation, the supernatant was replaced with 100 μl of fresh CCM medium in each well, and the cells were cultured for an additional 48 hours.
2. Tumor-specific TILs were added at an E:T ratio of 1:1. Specifically, after counting the tumor cells, TILs were diluted to 2×10⁵ cells/ml, and 100 μl of the cell suspension (resuspended in CCM) was added to each well. The culture was continued for 24 hours.
3. The steps for the MTT assay were the same as in Example 2.2. The oral cancer cells used in this experiment were adherent, while TILs were suspension cells. To detect oral cancer cell proliferation, the culture supernatant was collected into centrifuge tubes, and the adherent tumor cells were washed three times with PBS. The MTT assay was then performed on the viable adherent tumor cells at the bottom. The results are shown in FIG. 7.

### MTT Assay for Determination of T Cell Proliferation Stimulated by Virus Pre-Infected Primary Oral Cancer Cells:

The MOI was chosen as 0.01. The following groups were set up: CCM, TIL+CCM, TIL+OC1/2/3/4, TIL+OV-GFP, TIL+OV-OX40L, TIL+OV-IL-12, TIL+OV-OX40L/IL-12, TIL+OC1/2/3/4+OV-GFP, TIL+OC1/2/3/4+OV-OX40L, TIL+OC1/2/3/4+OV-IL-12, TIL+OC1/2/3/4+OV-OX40L/IL-12, TIL+OC1/2/3/4+OV-OX40L/αPD-1, TIL+OC1/2/3/4+OV-IL-12/αPD-1, TIL+OC1/2/3/4+OV-OX40L/IL-12/αPD-1 (OV-OX40L/αPD-1+OV-IL-12/αPD-1).

### Experimental Steps:

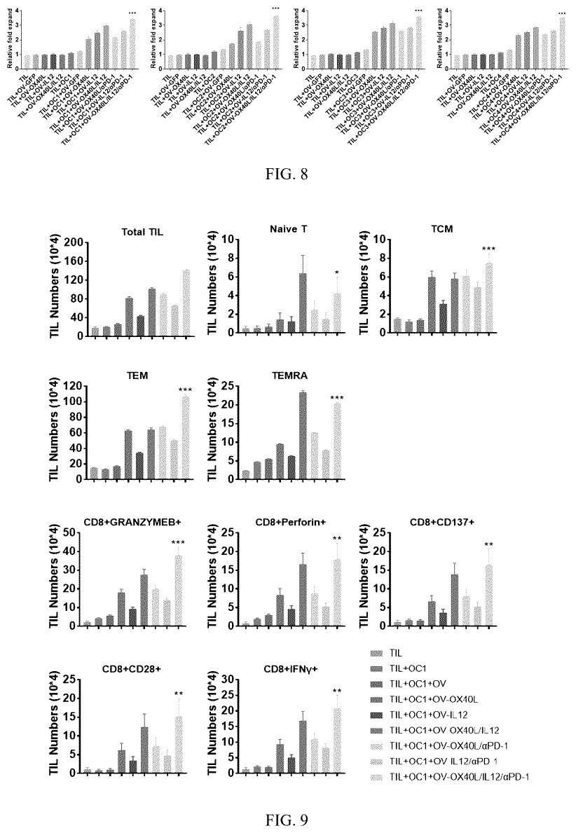

1. A 96-well plate was prepared, and 5,000 primary oral cancer cells were seeded per well. After 24 hours, the supernatant was discarded, and 100 μl of the prepared virus dilution (MOI=0.01) was added. The plate was centrifuged at 2,000 rpm for 10 minutes and then placed in the incubator for another 2 hours. After this incubation, the supernatant was replaced with 100 μl of fresh CCM medium per well, and the cells were cultured for an additional 48 hours.
2. After the 48-hours incubation, the supernatant was discarded, and the cells were washed three times with PBS. Tumor-specific TILs were then added at an E:T ratio of 1:1. Specifically, after counting the tumor cells, TILs were diluted to 2×10⁵ cells/ml, and 100 μl of the cell suspension (resuspended in CCM) was added to each well. The culture was continued for 24 hours.
3. The MTT assay steps were the same as in Example 2.2. To detect TIL proliferation in the culture supernatant, the supernatant and the three PBS wash solutions were collected, and the MTT assay was performed. The results are shown in FIG. 8.

### Results:

When TILs were co-cultured with oral cancer cells pre-infected with OV-OX40L/IL-12 for 24 hours, the viability of the oral cancer cells from the first case (OC1) was significantly reduced compared to the TIL-alone group (FIG. 5). This finding was further confirmed by an MTT assay conducted on the primary oral cancer cells from four cases, which results showed that TILs significantly inhibited the viability of oral cancer cells treated with OV-OX40L/IL-12/αPD-1 (FIG. 7). Subsequently, ELISA was used to assess the activation of TILs by primary oral cancer cells pre-infected with armed viruses. The results indicated that, compared to the OV-GFP+TIL group, primary oral cancer cells pre-infected with OV-OX40L/IL-12/αPD-1 significantly upregulated IFN-γ production in TILs, demonstrating that the expressed OX40L and IL-12 proteins effectively activated TILs (FIG. 6A). ELISPOT results showed that treating TILs with oral cancer cells pre-infected with OV-OX40L/IL-12/αPD-1 significantly increased the IFN-γ production in TILs and the IFN-γ expression in individual TIL cells, indicating that pre-infected cells not only increased the proportion of activated TILs but also significantly enhanced their tumor-killing ability (FIGS. 6B and 6C). Finally, the MTT assay was used to determine the proliferation of TILs in each group, and the results showed that TILs had the strongest proliferative ability in the OV-OX40L/IL-12/αPD-1+TIL group (FIG. 8). The above experiments demonstrate that primary oral cancer cells pre-infected with OV-OX40L/IL-12/αPD-1 significantly promoted the activation and expansion of TILs, enabling them to produce a potent anti-tumor effect.

### Example 3.2 Conversion of Oral Cancer Cells into APCs by Oncolytic Viruses

Flow Cytometry Demonstrated Conversion of Tumor Cells into APCs by Armed Oncolytic Viruses

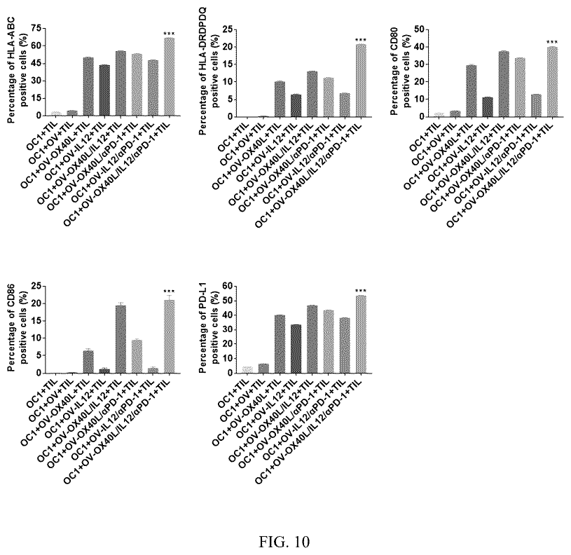

After TILs were co-cultured with the primary oral cancer cells pre-infected with various armed oncolytic viruses or their combinations, we used flow cytometry to detect the expression of APC-related genes on the surface of the primary oral cancer cells. We found that OV-OX40L/αPD-1+OV-IL-12/αPD-1 (OV-OX40L/IL-12/αPD-1) in combination with TILs upregulated the expression of the antigen presentation molecules HLA-A/B/C, HLA-DR/DP/DQ, CD80 and CD86 on the surface of the oral cancer cells. When oral cancer cells infected with OV-OX40L/IL-12/αPD-1 were co-cultured with TILs, the expression of PD-L1 on the oral cancer cells were also significantly increased (FIG. 10).

Additionally, we detected the expression of surface antigens on TILs using flow cytometry after co-culturing. The results indicated that oral cancer cells pre-infected with OV-OX40L/IL-12/αPD-1 significantly upregulated the expression of CD137 and PD-1 on TILs, demonstrating that the expression of OX40L, IL-12, and PD-1 scFv by these cancer cells effectively activates TILs (FIG. 9). FIG. 9 also illustrates the following: (1) Oral cancer cells infected with the armed oncolytic virus combination significantly increased the numbers of central memory and effector memory T cells, thereby effectively inhibiting tumor recurrence and metastasis; (2) Oral cancer cells infected with the armed oncolytic virus combination significantly upregulated the expression of granzyme B, perforin, and IFN-γ in CD8+ cells, as well as increase CD137 and CD28 expression, indicating that TILs can be robustly activated by the oral cancer cells infected with the oncolytic virus combination, and that the cytotoxic effects on oral cancer cells are mediated by granzyme B, perforin, and IFN-γ; (3) The number of PD-1 and TIM-3 positive cells did not differ significantly among the groups, suggesting that the combination strategy of this invention not only improves the efficiency of activation of TILs, but also mitigates the exhaustion of T cells.

The experiments mentioned above were carried out as follows. Briefly, the PerCP-Cy5.5-CD45 antibody was used for cell surface staining, with CD45+ cells being TILs and CD45-cells being primary oral cancer cells. Fluorescently labeled antibodies PE/DazzleTM 594 anti-human HLA-A,B,C, FITC anti-human HLA-DR, DP, DQ, PE anti-human CD80, FITC anti-human CD86, PE anti-human CD252 (OX40L), PE/DazzleTM 594 anti-human CD274 (B7-H1, PD-L1), as well as Alexa Fluor 700 anti-human CD137 (4-1BB) and PE/Cyanine7 anti-human CD279 (PD-1) antibodies were used to detect antigen expression on the surface of oral cancer cells and TILs by flow cytometry, respectively.
1. 500,000 primary oral cancer cells (OC1) were seeded into three six-well plates (16 wells total), with one duplicate well per group.
2. The oral cancer cells were pre-infected with engineered oncolytic viruses (MOI=0.01) for 48 hours. After the 48 hours infection, the supernatant was discarded, and 1×10⁶ TILs were added per well. The culture was continued for 24 hours, and then the cells were collected into 96-well U-bottom plates, centrifuged at 1,200 rpm for 3 minutes, and the supernatant was discarded.
3. 100 μL of Viability Dye (Zombie NIRTM Fixable Viability Kit, Biolegend) (diluted 1:1000 in PBS) was added to each well for staining dead cells. The plates were incubated in the dark at 4° C. for 10 minutes. Then, 100 μL of PBS was directly added, mixed well to wash off residual dye, centrifuged at 1,400 rpm at 4° C. for 5 minutes, and the supernatant was discarded.
4. Except for the blank control, 40 μL/well of the respective antibody mixtures (prepared as in Example 1.2) was added into the remaining wells, mixed well, and incubated in the dark at 4° C. for 1 hour. Flow cytometry analysis was then performed.
   qPCR Detected the Effect of Armed OV-OX40L/αPD-1 and OV-IL-12/αPD-1 Oncolytic Viruses on Surface Antigen Expression of Primary Oral Cancer Cells

The experimental groups included: OC1+TIL, OC1+OV-GFP+TIL, OC1+OV-OX40L+TIL, OC1+OV-IL-12+TIL, OC1+OV-OX40L/IL-12+TIL, OC1+OV-OX40L/αPD-1+TIL, OC1+OV-IL-12/αPD-1+TIL, OC1+OV-OX40L/IL-12/αPD-1 (OV-OX40L/αPD-1+OV-IL-12/αPD-1)+TIL.

The tumor cells (TC) from the first case (OC1) were seeded into three six-well plates, totaling 16 wells, with 500,000 cells per well and two duplicate wells per group. After 24 hours, the spent medium was replaced with 2 ml of CCM medium containing oncolytic viruses for each group. Following a 48-hour incubation, 1 million TILs were added for co-culture for an additional 24 hours before the cells were collected.

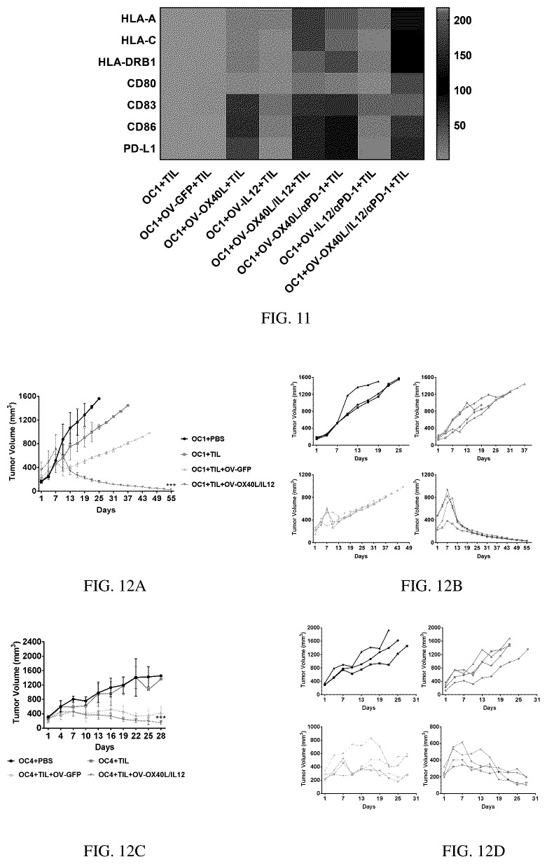

RNA was extracted and reverse-transcribed into cDNA. qPCR was then performed using fluorescently labeled sequence-specific primers to detect the expression of HLA-A, HLA-C, HLA-DRB1, CD80, CD86, and PD-L1 genes in the samples from each group. Among all groups, the combination of oncolytic viruses expressing OX40L, IL-12, and PD-1 scFv (all three genes) with TILs resulted in the highest expression levels of APC-associated genes (HLA-A, HLA-C, HLA-DRB1, CD86, and PD-L1). Relative to IL-12 and PD-1, OX40L played a more significant role in enhancing the expression of antigen presentation genes (HLA-A, HLA-C, HLA-DRB1, CD86, and PD-L1) in tumor cells (FIG. 11).

Experimental Procedure: RNA was extracted using the RNA Extraction Kit (Tiangen Biotech, DP430) according to the manufacturer's instructions. cDNA was synthesized using the HiScript 1st Strand cDNA Synthesis Kit (Vazyme Biotech), following the protocol provided by the manufacturer.

Primer sequence

| Primer | | Sequence | Tm(°C.) |
|---|---|---|---|
| HLA-A | F | TGTTCTAAAGTCCGCACGC | 58.48 |
| | R | TACCTCATGGAGTGGGAGC | 58.08 |
| HLA-C | F | CAGTTCGTGCGGTTCGACAG | 61.88 |
| | R | GCCTGGCGCTTGTACTTCTG | 61.64 |
| HLA-DRB1 | F | TGGTCCTGTCCTGTTCTCCA | 60.11 |
| | R | AGAAACGTGGTCTGGTGTCC | 59.89 |
| PD-L1 | F | TTGCTGAACGCCCCATACAA | 60.25 |
| | R | TCCAGATGACTTCGGCCTTG | 59.75 |
| CD80 | F | CTCAGAAGTGGAGTCTTACCCTG | 59.81 |
| | R | TGTTCCTGGGTCTCCAAAGG | 59.23 |
| CD83 | F | CGCCCACTTGTCCCACTATC | 60.46 |
| | R | CATTAGCCCATGCAACAGCC | 59.9 |
| CD86 | F | TAGCACAGACACACGGATGAG | 59.8 |
| | R | ACTGAAGTTAGCAGAGAGCAGG | 59.77 |

50 μl of a reaction mixture for PCR was prepared:

| | Volume⁽¹⁾ | | |
|---|---|---|---|
| Reagent | (50 μl reaction) | | Final Conc. |
| FastStart Universal SYBR Green Master | 25 | μl | 1x |
| (ROX) | | | |
| Forward Primer (30 μM) | 0.5 | μl | 300 nM |
| Reverse Primer (30 μM) | 0.5 | μl | 300 nM |
| Water, PCR Grade | 19.0 | μl | |
| Total Volume | 45 | μl | |

After carefully mixing the solution, 45 μl of the PCR mixture was pipetted into the PCR microplate wells. Then, 5 μl of template DNA was added. The qPCR operation was performed according to the instrument instructions. The qPCR amplification conditions were as follows: pre-denaturation at 93° C. for 2 minutes, followed by 40 cycles of 93° C. for 1 minute, 55° C. for 1 minute, 72° C. for 1 minute, and a final extension at 72° C. for 7 minutes.

### Example 3.3 In Vivo Animal Model Experiments

The studies mentioned above demonstrated that the combination of OV-OX40L/αPD-1+IL-12/αPD-1 with TILs enhances tumor-killing and T cell activation in vitro. To further validate these findings in vivo, we evaluated the efficacy of oncolytic viruses, TILs, and the combination therapy in oral cancer PDX models and in immunocompetent mice with grafted tumors.

### Example 3.3.1 Immunodeficient Mouse Tumor Model Experiments

### Establishment and Passage of Oral Cancer PDX Models

(1) Establishment of primary PDX models:
   \textcircled{1} The samples obtained from oral cancer patients were cut into 2×2×2 mm³ tissue blocks using surgical scissors and placed in sterile tissue culture medium (RPMI 1640).
   \textcircled{2} Mice were anesthetized by intraperitoneal injection of 4% chloral hydrate (Adamas). The hair from the right axillary region was removed, and the mice were fixed in a supine position on a super-clean bench. The area was disinfected with 70% alcohol, and an incision about 3 mm long was made 2 cm away from the mouse axilla using scissors. Blunt forceps were used to subcutaneously implant three small blocks of oral cancer tissue (immersed in Basement Membrane Matrix (Corning, 354234)). The wound was sutured with sterile sutures to prevent the tumor blocks from falling out, establishing the primary PDX animal model, referred to as the P0 generation.
   \textcircled{3} Four NSG mice were used for the initial passage of each tumor sample. After implantation, the size of the xenografts was measured every three days using a caliper. The tumor volume was calculated using the formula: V=length×(width)²/2.
   \textcircled{4} The remaining oral cancer tissue blocks were frozen and stored in cryovials.
(2) Passage of PDX models
   \textcircled{1} When the subcutaneous tumors in the P0 generation mice grew to about 1,000 mm³, the mice were euthanized by cervical dislocation. Sterile towels were laid out, and the skin around the mouse axillae was disinfected with 70% alcohol. A sterile surgical blade was used to cut open the skin around the tumor, which was then removed and placed in a sterile culture dish. A portion of the tissue was fixed in 4% paraformaldehyde solution (Solarbio), and the remaining tumor was divided into tissue blocks of about 0.2 cm×0.2 cm×0.2 cm using sterile instruments.
   \textcircled{2} Five NSG mice aged 5 weeks were used to implant the tissue blocks as described above, establishing the first generation of the PDX animal model, referred to as the P1 generation. The weight of the mice and the tumor volume were monitored weekly, and a tumor growth curve was plotted. When the subcutaneous tumors in the P1 generation mice grew to about 1,000 mm³, the tumors were passaged using this method to establish the second, third, and fourth generations of the PDX animal model, referred to as the P2, P3, and P4 generations.
      Evaluation of the Inhibitory Effect of OV-OX40L/IL-12 Combined with TILs on the Growth of OC1- and OC4-PDX Tumors

Oral cancer tissues from two cases (OC1 and OC4) were used to establish PDX models. The inhibitory effect of the oncolytic virus combined with TILs on tumor growth was evaluated in the fourth and fifth generations of the PDX models. Before injecting TILs into the tumors, the TILs isolated and expanded according to Example 1 were co-cultured with tumor cells stimulated by the DEC cocktail for 24 hours to activate the TILs. The results are shown in FIGS. 12A-12B and 12C-12D.

PDX Model of Oral Cancer from the First Case:

The OC1-PDX model was established, and when the tumors grew to 200-300 mm3, the mice were randomly divided into four groups, with five mice per group, to receive the following treatments: OC1+PBS, OC1+TIL, OC1+OV-GFP+TIL, and OV-OX40L/IL-12+TIL.

Oncolytic Virus Administration: On day 0, mice in the OC1+OV-GFP+TIL and OC1+OV-OX40L/IL-12+TIL groups received a single intratumoral injection of 2×10⁵ PFU (50 μL) of the virus at the tumor site.

TIL Administration: On day 2, mice in the OC1+TIL, OC1+OV-GFP+TIL, and OC1+OV-OX40L/IL-12+TIL groups received a single intratumoral injection of 2×10⁶ TILs at the tumor site.

Mice in the OC1+TIL, OC1+OV-GFP+TIL, and OC1+OV-OX40L/IL-12+TIL groups received an intraperitoneal injection of 10 μg/100 μL Super-IL-2 protein every other day from day 2 to day 18.

The size of the xenografts was measured every three days using a caliper, and the tumor volume was calculated using the formula: V=length×(width)²/2.

PDX Model of Oral Cancer from the Fourth Case:

The OC4-PDX model was established, and when the tumors grew to 200-300 mm³, the mice were randomly divided into four groups, with five mice per group, to receive the following treatments: OC4+PBS, OC4+TIL, OC4+OV-GFP+TIL, and OC4+OV-OX40L/IL-12+TIL.

The treatment regimen, tumor volume measurement, and calculation method were the same as described above.

### Detection of IFN-γ Expression Levels in the Tumor Microenvironment Using ELISA

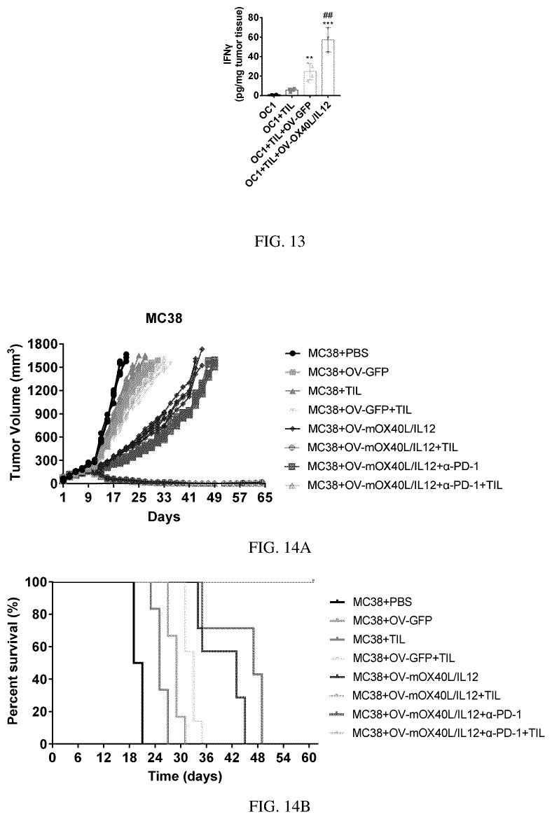

From the four treatment groups of the OC1-PDX model, two to three mice were randomly selected from each group, and tumor tissue blocks were taken and frozen on day 7. The groups were as follows: OC1 (two mice, two blocks each), OC1+TIL (two mice, two blocks each), OC1+OV-GFP+TIL (two mice, two blocks each), and OC1+OV-OX40L/IL-12+TIL (three mice, one block each). The expression level of IFN-γ in the tumor blocks was detected using the following steps:
\textcircled{1} The frozen tissue blocks were removed from liquid nitrogen, and tissue blocks of appropriate size were randomly selected and weighed.
\textcircled{2} A syringe and cell strainer were used to grind the selected tissue blocks from each group into a homogeneous suspension. The suspension was washed with PBS 2-3 times, using a mass-to-volume ratio of 10:1 (e.g., for an 8 mg tissue block, the volume was adjusted to 0.8 ml).
\textcircled{3} The filtrate was collected in 1.5 ml EP tubes, centrifuged at 400 g for 5 minutes, and the supernatant was filtered through a 0.45 μm membrane into a new EP tube for testing. The Human IFN-γ Valukine ELISA Kit (R&D Systems, VAL104) was used according to the manufacturer's instructions to determine the IFN-γ content in the supernatant. The results are shown in FIG. 13.

### Results:

TIL monotherapy only moderately delayed tumor growth in the first patient's PDX model and could not alleviate the tumor burden at the end of treatment. The combination therapy of OV-GFP with TILs exhibited some tumor inhibition compared to TIL monotherapy, but the tumor burden in the PDX model remained substantial at the end of treatment. The combination therapy of OV-OX40L/IL-12 with TILs significantly reduced the tumor burden in the first patient's PDX model, and the mice in this group were completely cured after 7 weeks of treatment (FIGS. 12A-12B). FIG. 12A shows the tumor growth curves for each group; FIG. 12B is an expanded view of FIG. 12A, displaying the individual tumor growth curves for each mouse in each group.

TIL monotherapy had almost no therapeutic effect on the fourth patient's PDX model. The oncolytic virus OV-OX40L/IL-12 alone moderately inhibited the growth of the fourth patient's PDX, but the inhibitory effect was not significant. Compared to TIL monotherapy, the combination of OV-OX40L/IL-12 with TILs significantly inhibited the growth of the fourth case's PDX model (FIGS. 12C and 12D). FIG. 12C shows the average tumor growth curves for each group; FIG. 12D is an expanded view of FIG. 12C, displaying the individual tumor growth curves for each mouse in each group.

To demonstrate that the inhibitory effect of this combination therapy on oral cancer PDX models was related to the activation of the adoptively transferred TILs, the IFN-γ content in the tumor homogenates from each group was detected by ELISA. The results showed that the combination therapy of OV-OX40L/IL-12 with TILs significantly upregulated the activation level of the adoptively transferred TILs in the tumors (FIG. 13).

TIL monotherapy only moderately delayed tumor growth in the first patient's PDX model and did not alleviate the tumor burden by the end of treatment. The combination therapy of OV-GFP with TILs showed some tumor inhibition compared to TIL monotherapy, but the tumor burden remained substantial at the end of treatment. The combination therapy of OV-OX40L/IL-12 with TILs significantly reduced the tumor burden in the first patient's PDX model, with mice in this group being completely cured after 7 weeks of treatment (FIGS. 12A-12B). FIG. 12A shows the tumor growth curves for each group; FIG. 12B is an expanded view of FIG. 12A, displaying the individual tumor growth curves for each mouse in each group.

In the fourth patient's PDX model, TIL monotherapy had almost no therapeutic effect. The oncolytic virus OV-OX40L/IL-12 alone moderately inhibited tumor growth, but the effect was not significant. The combination of OV-OX40L/IL-12 with TILs significantly inhibited tumor growth compared to TIL monotherapy (FIGS. 12C and 12D). FIG. 12C shows the average tumor growth curves for each group, and FIG. 12D is an expanded view of FIG. 12C, displaying the individual tumor growth curves for each mouse in each group.

To demonstrate that the inhibitory effect of this combination therapy on oral cancer PDX models was related to the activation of adoptively transferred TILs, the IFN-γ content in tumor homogenates from each group was measured by ELISA. The results indicated that the combination therapy of OV-OX40L/IL-12 with TILs significantly upregulated the activation level of adoptively transferred TILs in the tumors (FIG. 13).

### Example 3.3.2 Immunocompetent Mouse Tumor Model Experiments

In the following experiments, the oncolytic viruses OV-mOX40L and OV-mIL-12 were constructed using mouse OX40L and mouse IL-12, respectively, following the methods described for armed oncolytic viruses. TILs were isolated and expanded from the grafted tumors in the respective mice according to the previously described TIL preparation method. Additionally, an anti-PD-1 antibody protein (purchased from BioXcell, catalog number BE0146) was used instead of the PD-1 antibody expressed by oncolytic viruses, to investigate the effects of different administration forms of the PD-1 antibody in combination with armed oncolytic viruses expressing OX40 and IL-12 in TIL therapy.

### 1. Establishment of MC38 Colon Cancer Cell Line Transplanted Tumor Model

Immunocompetent C57BL/6J mice (Vitalriver) were inoculated with 1×10⁶ MC38 cells on one side to establish the graft tumor model. When the tumor volume reached 50 mm³, the mice were randomly divided into the following 8 groups, with 6 mice per group:

$$Groups : MC 38 + PB S , M C 38 + OV \text{-} GFP , \\ MC 38 + TIL , M C 38 + OV \text{-} GFP + T IL , \\ MC 38 + OV \text{-} mOX 40 L \text{/} mIL \text{-} 12 , \\ MC 38 + OV \text{-} mOX 40 L \text{/} mIL \text{-} 12 + TIL , \\ MC 38 + OV \text{-} mOX 40 L \text{/} mIL \text{-} 12 + α \text{-} PD \text{-} 1 , \\ MC 38 + OV \text{-} mOX 40 L \text{/} mIL \text{-} 12 + α \text{-} PD \text{-} 1 + TIL .$$

The day of randomization was designated as day 1. Subsequently, the mice were treated with oncolytic viruses, TILs, and/or anti-PD-1 antibody protein according to their respective groups, following the following administration regimen:

Oncolytic Viruses (OV-mOX40L and OV-mIL-12): On days 3 and 5, OV-mOX40L and OV-mIL-12 were injected intratumorally for a total of two treatments, with each mouse receiving an injection of 2×10⁶ PFU (100 μL, with a 1:1 ratio of OV-mOX40L and OV-mIL-12) per treatment.

TILs: Starting on day 7, each mouse received an intratumoral injection of 1×10⁶ TILs resuspended in PBS, with an injection volume of 100 μL.

Anti-PD-1 antibody: From day 7, the last two groups received an intraperitoneal injection of 10 mg/kg α-PD-1 every two weeks, for a total of two injections.

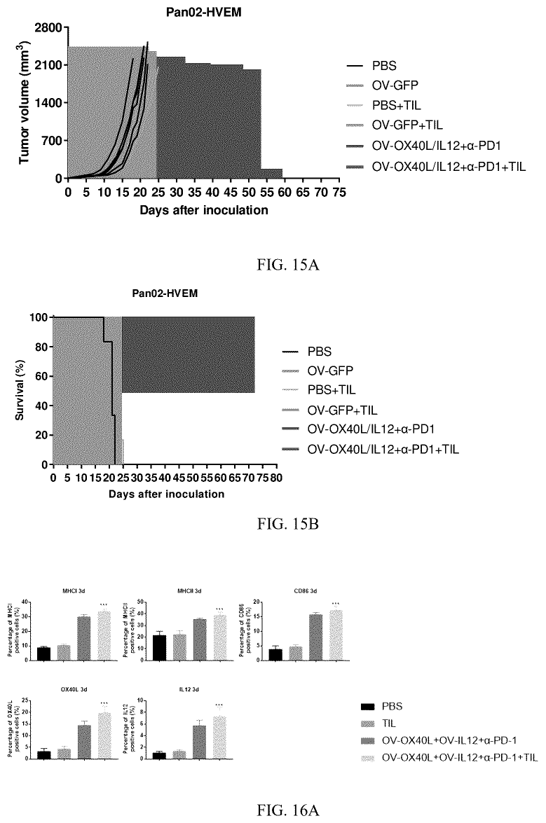

The tumor volume and body weight of the mice were recorded every two days, and the growth of the graft tumors was monitored and plotted. When the tumor volume reached 1500 mm³, the mice were euthanized, and samples were collected. The results are shown in FIG. 14.
(1) Compared to the PBS control group, TIL monotherapy only slightly delayed the growth of the MC38 transplanted tumors and did not alleviate the tumor burden in mice.
(2) Compared to the OV-GFP monotherapy group, the tumor growth rate was significantly slower in mice treated with OV-mOX40L+OV-mIL-12 or OV-mOX40L+OV-mIL-12+a-PD-1. The a-PD-1 antibody enhanced the anti-tumor effect of OV-mOX40L+OV-mIL-12 to a certain extent.
(3) The introduction of TILs significantly enhanced the therapeutic effect of OV-mOX40L and OV-mIL-12 in the MC38 transplanted tumors. On day 16 after combination treatment, the tumor volumes of 7 mice in the OV-mOX40L+OV-mIL-12+TIL group were maintained at around 30-50 mm³, while the tumor volumes of 7 mice in the OV-mOX40L+OV-mIL-12+a-PD-1+TIL group were around 20-40 mm³, showing a 26% reduction in average tumor volume.

### 2. Pan02-HVEM Pancreatic Cancer Cell Line Transplanted Tumors

Immunocompetent C57BL/6J mice were inoculated with 5×10⁵ Pan02-HVEM cells on one side to establish the transplanted tumor model (The Pan02-HVEM cell line was constructed by introducing the HVEM receptor for virus into the mouse pancreatic cancer cell line Pan02 cells to enable effective infection of the cancer cells by the HSV-1-based oncolytic virus). When the tumor volume reached 50 mm³, the mice were randomly divided into the following 6 groups, with 8 mice per group:

$$Groups : Pan 02 \text{-} HVEM + PBS , \\ Pan 0 \; 2 \text{-} HVEM + OV \text{-} GFP , \\ Pan 02 \text{-} HVEM + TIL , \\ Pan 02 \text{-} HVEM + OV \text{-} GFP + TIL , \\ Pan 02 \text{-} HVEM + OV \text{-} mOX 40 L \text{/} IL \text{-} 12 \text{/} α \text{-} PD \text{-} 1 , \\ Pan 02 \text{-} HVEM + OV \text{-} mOX 40 L \text{/} IL \text{-} 12 \text{/} α \text{-} PD \text{-} 1 + TIL .$$

The day of randomization was designated as day 1. Subsequently, the mice were treated with oncolytic viruses, TILs, and/or anti-PD-1 antibody protein according to their respective groups, following the following administration regimen:

Oncolytic Viruses (OV-mOX40L and OV-mIL-12): On days 3 and 5, OV-mOX40L and OV-mIL-12 were injected intratumorally for a total of two treatments, with each mouse receiving an injection of 2×10⁶ PFU (100 μL, with a 1:1 ratio of OV-mOX40L and OV-mIL-12) per treatment.

TILs: Starting on day 7, each mouse received an intratumoral injection of 1×10⁶ TILs resuspended in PBS, with an injection volume of 100 μL.

Anti-PD-1 antibody: From day 5, the last two groups received an intraperitoneal injection of 10 mg/kg α-PD-1 every two weeks, with a total of two injections.

The tumor volume and body weight of the mice were recorded every two days, and the growth of the transplanted tumors was monitored and plotted. When the tumor volume reached 1500 mm³, the mice were euthanized, and samples were collected. The results are shown in FIG. 15:
(1) Compared to the PBS control group, TIL monotherapy did not significantly inhibit the growth of the Pan02-HVEM transplanted tumors.
(2) Compared to the OV-GFP monotherapy group, the tumor growth rate was significantly slower in mice treated with OV-mOX40L+OV-mIL-12+PD-1 scFv. After 22 days of combination treatment, the tumors completely disappeared in 4 out of 7 mice.
(3) The introduction of TILs had a certain boosting effect in the OV-mOX40L+OV-mIL-12+PD-1 scFv+TIL treatment group. This boosting effect of the TILs appeared to be smaller in the pancreatic tumor-bearing mice compared to the effect observed for TILs in the colon cancer tumor-bearing mice treated with the armed oncolytic virus (OV-mOX40L/mIL-12/a-PD-1). This difference is likely because Pan02 pancreatic tumors already contain a high abundance of immune cells, which limits the additional benefit of introducing TILs.

### 3. Analysis of Immune Cells in Tumors and Spleens

Pan02-HVEM cells were inoculated in C57BL/6J mice to establish transplanted tumors. Samples were collected on days 3 and 7 after TIL therapy.

Spleen cell isolation: The spleen was cut into pieces and ground through a 70 μm cell strainer, washed with PBS three times, and the cells were collected in a 50 ml centrifuge tube for centrifugation and red blood cell lysis.

Steps for isolating cells from subcutaneous tumors:
(1) The mice were euthanized, immersed in 70% ethanol, and the tumors were removed.
(2) The tumors were washed with PBS to remove blood vessels and blood. The tumors were weighed and photographed.
(3) The tumor was divided into two halves. One half was fixed with 4% paraformaldehyde for sectioning and immunohistochemical staining.
(4) The other half of the tumor was minced with scissors and placed in 8 ml of digestion solution (FACS buffer: PBS+2% FBS, containing collagenase I (1 mg/ml), dispase II (0.05 mg/ml) or hyaluronidase (1 mg/ml), and DNase (0.5 mg/ml)). It was then placed in an incubator at 37° C. with shaking for about one hour.
(5) After digestion, the digest was filtered through a 70 μm cell strainer.
(6) The cells were centrifuged at 1400 rpm, 4° C. for 5 minutes, and the supernatant was discarded. The pellet was resuspended by vortexing, and 20 ml of DMEM was added for washing. 40% Percoll and 70% Percoll solutions were prepared. 6 mL of 70% Percoll was added to a 15 ml centrifuge tube in advance. The pellet from step 6 was resuspended in 40% Percoll and slowly layered on top of the 70% Percoll, to form a distinct interface, and then centrifuged for 30 minutes. To prepare 100 ml of 40% Percoll: Mix 4 ml of 10x PBS, 36 ml of Percoll, and 60 ml of DMEM. To prepare 90 ml of 70% Percoll: Mix 7 ml of 10x PBS, 63 ml of Percoll, and 30 ml of DMEM.
(7) The viscous upper layer was removed carefully (ensuring not to disturb the middle layer of cells). The white blood cells were collected from the middle layer (tumor cells at the bottom) and transferred to a 15 ml centrifuge tube containing 10 ml of DMEM, and centrifuged. If the cell pellet was very red, the red blood cells were lysed using ACK lysis buffer. An appropriate amount (typically 3 mL) of lysis buffer was added based on the pellet volume, vortexed, incubated at room temperature for 4 minutes, and then centrifuged at 1400 rpm, 4° C. for 5 minutes.
(8) The supernatant was discarded. The pellet was resuspended in DMEM, with the volume of DMEM adjusted to ensure consistent cell density across all samples. The suspension was filtered through a cell strainer and transferred to a new flow cytometry tube. Estimate The amount of DMEM to add was estimated based on the smallest tumor volume, to ensure uniform density for all samples.

The flow cytometry procedure followed similar steps as described in Example 3.2 for sample analysis.

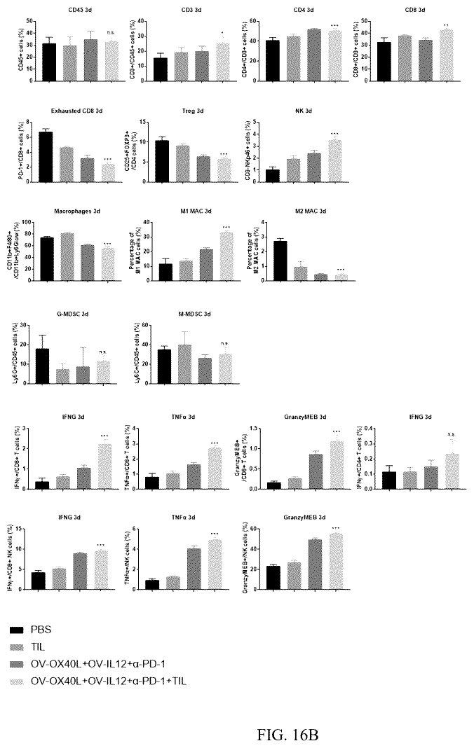

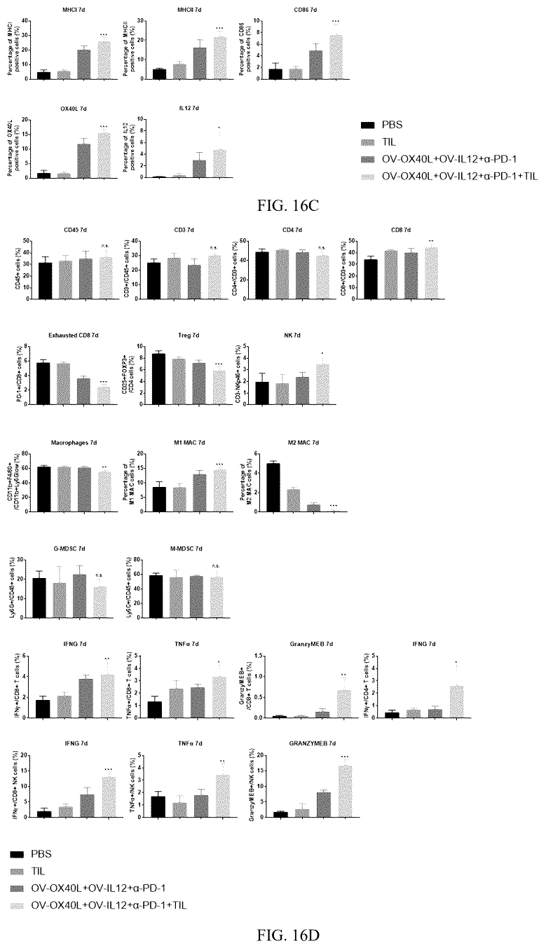

Flow cytometry confirmed that OV-mOX40L/IL-12 significantly increased the proportion of CD8+ T cells in the tumor and spleen, along with a marked increase in the expression of IFN-γand Granzyme B. The results are shown in FIG. 16.

FIG. 16A: This figure illustrates the expression of surface and intracellular markers on tumor cells of tumor tissues in different treatment groups on day 3 post-treatment. Compared to TIL monotherapy or oncolytic virus monotherapy groups, the combination therapy group significantly upregulated the expression of MHC I, MHC II, CD86, OX40L, and IL-12 in tumor cells, suggesting that the combination of the oncolytic virus and TILs can transform tumor cells into antigen-presenting cells.

FIG. 16B: This figure displays the proportions of various immune cell populations and marker expressions in tumor tissues across different treatment groups on day 3 post-treatment. The combination therapy group, compared to TIL monotherapy or oncolytic virus monotherapy groups, significantly increased the proportions of CD3+ T cells, CD8+ T cells, NK cells, and M1 macrophages in tumor tissues, while significantly decreasing the proportions of exhausted CD8+ T cells, Tregs, and M2 macrophages. The proportions of CD45+ TILs, CD4+ T cells, macrophages, G-MDSCs, and M-MDSCs remained largely unchanged.

Additionally, compared to TIL monotherapy or oncolytic virus monotherapy groups, the combination therapy group significantly upregulated the expression of IFN-γ, TNF-α, and Granzyme B in CD8+ T cells and NK cells within tumor tissues, although IFN-γ expression in CD4+ T cells did not show a significant difference.

These results indicate that the combination of the oncolytic virus and TILs can significantly enhance the proportions and cytotoxic capabilities of CD8+ T cells, NK cells, and M1 macrophages in tumor tissues, while reducing the infiltration of immunosuppressive cells such as exhausted CD8+ T cells, Tregs, and M2 macrophages.

FIG. 16C: This figure illustrates the expression of surface and intracellular markers on tumor cells of tumor tissues in different treatment groups on day 7 post-treatment. The results indicate that, compared to TIL monotherapy or oncolytic virus monotherapy groups, the combination therapy group significantly upregulated the expression of MHC I, MHC II, CD86, and OX40L in tumor cells. This further confirms that the combination of the oncolytic virus and TILs can transform tumor cells into antigen-presenting cells.

FIG. 16D: This figure presents the proportions of various immune cell populations and marker expressions in tumor tissues across different treatment groups on day 7 post-treatment. The combination therapy group, compared to TIL monotherapy or oncolytic virus monotherapy groups, significantly increased the proportions of CD8+ T cells, NK cells, and M1 macrophages in tumor tissues, while significantly decreasing the proportions of exhausted CD8+ T cells, Tregs, and M2 macrophages. The proportions of CD45+ TILs, CD3+ T cells, CD4+ T cells, macrophages, G-MDSCs, and M-MDSCs remained largely unchanged.

Additionally, compared to TIL monotherapy or oncolytic virus monotherapy groups, the combination therapy group significantly upregulated the expression of IFN-γ, TNF-α, and Granzyme B in CD8+ T cells and NK cells within tumor tissues.

These results suggest that the combination of oncolytic virus and TILs can significantly enhance the proportions and cytotoxic capabilities of CD8+ T cells, NK cells, and M1 macrophages in tumor tissues, while reducing the infiltration of immunosuppressive cells such as exhausted CD8+ T cells, Tregs, and M2 macrophages.

### SEQUENCE LISTING

Gene sequence encoding IL-12 P40
SEQ ID NO: 1
ATGTGTCACCAGCAGTTGGTCATCTCTTGGTTTTCCCTGGTTTTTCTGGCATCTCCC
CTCGTGGCCATATGGGAACTGAAGAAAGATGTTTATGTCGTAGAATTGGATTGGTA
TCCGGATGCCCCTGGAGAAATGGTGGTCCTCACCTGTGACACCCCTGAAGAAGAT
GGTATCACCTGGACCTTGGACCAGAGCAGTGAGGTCTTAGGCTCTGGCAAAACCC
TGACCATCCAAGTCAAAGAGTTTGGAGATGCTGGCCAGTACACCTGTCACAAAGG
AGGCGAGGTTCTAAGCCATTCGCTCCTGCTGCTTCACAAAAAGGAAGATGGAATT
TGGTCCACTGATATTTTAAAGGACCAGAAAGAACCCAAAAATAAGACCTTTCTAA
GATGCGAGGCCAAGAATTATTCTGGACGTTTCACCTGCTGGTGGCTGACGACAAT
CAGTACTGATTTGACATTCAGTGTCAAAAGCAGCAGAGGCTCTTCTGACCCCCAA
GGGGTGACGTGCGGAGCTGCTACACTCTCTGCAGAGAGAGTCAGAGGGGACAAC
AAGGAGTATGAGTACTCAGTGGAGTGCCAGGAGGACAGTGCCTGCCCAGCTGCT
GAGGAGAGTCTGCCCATTGAGGTCATGGTGGATGCCGTTCACAAGCTCAAGTATG
AAAACTACACCAGCAGCTTCTTCATCAGGGACATCATCAAACCTGACCCACCCAA
GAACTTGCAGCTGAAGCCATTAAAGAATTCTCGGCAGGTGGAGGTCAGCTGGGA
GTACCCTGACACCTGGAGTACTCCACATTCCTACTTCTCCCTGACATTCTGCGTTC
AGGTCCAGGGCAAGAGCAAGAGAGAAAAGAAAGATAGAGTCTTCACGGACAAG
ACCTCAGCCACGGTCATCTGCCGCAAAAATGCCAGCATTAGCGTGCGGGCCCAGG
ACCGCTACTATAGCTCATCTTGGAGCGAATGGGCATCTGTGCCCTGCAGTTAG
Gene sequence encoding IL-12 P35
SEQ ID NO: 2
ATGTGGCCCCCTGGGTCAGCCTCCCAGCCACCGCCCTCACCTGCCGCGGCCACAG
GTCTGCATCCAGCGGCTCGCCCTGTGTCCCTGCAGTGCCGGCTCAGCATGTGTCC
AGCGCGCAGCCTCCTCCTTGTGGCTACCCTGGTCCTCCTGGACCACCTCAGTTTGG
CCAGAAACCTCCCCGTGGCCACTCCAGACCCAGGAATGTTCCCATGCCTTCACCA
CTCCCAAAACCTGCTGAGGGCCGTCAGCAACATGCTCCAGAAGGCCAGACAAAC
TCTAGAATTTTACCCTTGCACTTCTGAAGAGATTGATCATGAAGATATCACAAAAG
ATAAAACCAGCACAGTGGAGGCCTGTTTACCATTGGAATTAACCAAGAATGAGAG
TTGCCTAAATTCCAGAGAGACCTCTTTCATAACTAATGGGAGTTGCCTGGCCTCCA
GAAAGACCTCTTTTATGATGGCCCTGTGCCTTAGTAGTATTTATGAAGACTTGAAG
ATGTACCAGGTGGAGTTCAAGACCATGAATGCAAAGCTTCTGATGGATCCTAAGA
GGCAGATCTTTCTAGATCAAAACATGCTGGCAGTTATTGATGAGCTGATGCAGGCC
CTGAATTTCAACAGTGAGACTGTGCCACAAAAATCCTCCCTTGAAGAACCGGATT
TTTATAAAACTAAAATCAAGCTCTGCATACTTCTTCATGCTTTCAGAATTCGGGCAG
TGACTATTGATAGAGTGATGAGCTATCTGAATGCTTCCTAA
Sequence of the gene encoding Trimeric OX40L
SEQ ID NO: 3
ACCAGGATAAGATCGAGGCTCTGTCCTCCAAGGTGCAGCAGCTGGAACGGTCCAT
CGGCCTGAAGGACCTGGCCATGGCTGACCTGGAACAGAAAGTGCTGGAAATGGA
AGCCTCCACACAGGTATCACATCGGTATCCTCGAATCCAAAGTATCAAAGTACAATT
TACCGAATATAAGAAGGAGAAAGGTTTCATCCTCACTTCCCAAAAGGAGGATGAA
ATCATGAAGGTGCAGAACAACTCAGTCATCATCAACTGTGATGGGTTTTATCTCAT
CTCCCTGAAGGGCTACTTCTCCCAGGAAGTCAACATTAGCCTTCATTACCAGAAGG
ATGAGGAGCCCCTCTTCCAACTGAAGAAGGTCAGGTCTGTCAACTCCTTGATGGT
GGCCTCTCTGACTTACAAAGACAAAGTCTACTTGAATGTGACCACTGACAATACCT
CCCTGGATGACTTCCATGTGAATGGCGGAGAACTGATTCTTATCCATCAAAATCCT
GGTGAATTtTGTGTCCTT
Sequence of the gene encoding PD-1 scFv VH
SEQ ID NO: 4
CAGGTGCAGCTGGTGGAGAGCGGCGGCGGCGTGGTGCAGCCCGGCAGGAGCCTG
AGGCTGGACTGCAAGGCCAGCGGCATCACCTTCAGCAACAGCGGCATGCACTGG
GTGAGGCAGGCCCCCGGCAAGGGCCTGGAGTGGGTGGCCGTGATCTGGTACGAC
GGCAGCAAGAGGTACTACGCCGACAGCGTGAAGGGCAGGTTCACCATCAGCAGG
GACAACAGCAAGAACACCCTGTTCCTGCAGATGAACAGCCTGAGGGCCGAGGAC
ACCGCCGTGTACTACTGCGCCACCAACGACGACTACTGGGGCCAGGGCACCCTGG
TGACCGTG
Sequence of the gene encoding PD-1 scFv VL
SEQ ID NO: 5
GAGATCGTGCTGACCCAGAGCCCCGCCACCCTGAGCCTGAGCCCCGGCGAGAGG
GCCACCCTGAGCTGCAGGGCCAGCCAGAGCGTGAGCAGCTACCTGGCCTGGTAC
CAGCAGAAGCCCGGCCAGGCCCCCAGGCTGCTGATCTACGACGCCAGCAACAGG
GCCACCGGCATCCCCGCCAGGTTCAGCGGCAGCGGCAGCGGCACCGACTTCACC
CTGACCATCAGCAGCCTGGAGCCCGAGGACTTCGCCGTGTACTACTGCCAGCAGA
GCAGCAACTGGCCCAGGACCTTCGGCCAGGGCACCAAGGTGGAG
CMV Promoter
SEQ ID NO: 6
CGTTACATAACTTACGGTAAATGGCCCGCCTGGCTGACCGCCCAACGACCCCCGC
CCATTGACGTCAATAATGACGTATGTTCCCATAGTAACGCCAATAGGGACTTTCCAT
TGACGTCAATGGGTGGAGTATTTACGGTAAACTGCCCACTTGGCAGTACATCAAGT
GTATCATATGCCAAGTACGCCCCCTATTGACGTCAATGACGGTAAATGGCCCGCCT
GGCATTATGCCCAGTACATGACCTTATGGGACTTTCCTACTTGGCAGTACATCTACG
TATTAGTCATCGCTATTACCATGGTGATGCGGTTTTGGCAGTACATCAATGGGCGTG
GATAGCGGTTTGACTCACGGGGATTTCCAAGTCTCCACCCCATTGACGTCAATGGG
AGTTTGTTTTGGCACCAAAATCAACGGGACTTTCCAAAATGTCGTAACAACTCCG
CCCCATTGACGCAAATGGGCGGTAGGCGTGTACGGTGGGAGGTCTATATAAGCAG
AGCT
SV40 polyA
SEQ ID NO: 7
GATCCAGACATGATAAGATACATTGATGAGTTTGGACAAACCACAACTAGAATGCA
GTGAAAAAAATGCTTTATTTGTGAAATTTGTGATGCTATTGCTTTATTTGTAACCAT
TATAAGCTGCAATAAACAAGTT
Homologyarm-1L
SEQ ID NO: 8
tccacgtcgaaggcttttgcattgtaaagctacccgcctacccgcgcctcccaataaaaaaagaacatacaccaatgggtcttatttggt
attacctggtttatttaaaaagatatacagtaagacatcccatggtaccaaagaccggggcgaatcagcgggcccccatcatctgagag
acgaacaaatcggcggcgcgggccgtgtcaacgtccacgtgtgctgcgctgctggcgttgacaagggccccggcctccgcgttgga
tgcctccggttgggatccggtggcggcgggggggaacgcgggctccgtcggtagaggggcgcgcgtctgggtggaaggacatgg
gggcggtggcgggcctgggggcaggcagctggggcatacgggggagtgggggcatgggacgccggaccctggggaggacc
gtagggggcgctgtgtggtggggggcgatacacggcctccgggggacaaagggccgggtgggtcgttgttggttccggctccccc
acctgagggcgatagtgcgccaccggcgtgtacattccatagggggcgctggtccgagcccgcatgtgcgccagttcctgctgcag
agacgtcaccgcccccatcagcgccgtgatggtctcgttggtcccgggagactggcgggccgcgcgccgagagtcgaccccgcgc
ggcccgcctcgagcctccccggggtagtacgggtagtccgcgtccggttcgtcctggtcgcagtaggactccgacggccccgcctc
gtaccggcgacgctttcccgacccccggaccccagggtctcccgcggccgtctgaccgcccgcctggcggtccgcggctatggcc
cccaccaacgcggctatctgcgcctcgagtgggctgggtcccgagaacagcaccccgggatactgatgggcgacgtggggagggt
aatgctgggaaagacccgcgccgtgaggcccataggccacggcccccgccgcagccgggaaaccaaacgcggaatgcggctgg
ggttggggcgcggcgtggccggcgacgagctggttgtaatgggaggccgggatccacaggtagctcccgtcgccgggcggcgcg
ggggccggggtgcccgatgccggaacggggttcatggggggcagtaccgggggcgaggcgaccccgcgctgacggacgattg
ggcaccggtcaccctgcggcgcggcagcgatcgagccgggcggtatgtccgtggactccggggccccgttcttatacccgcgcgc
cggcgcggaaacaggctccgccccccacattttgaatttttcgctcgcctggaggtaggtgtgtccggcgatcccggcctgccgccgc
cgctcggccaccaggctccagcggtcccgcagcatcatgttgttaacggcggtgG
Homologyarm-1R
SEQ ID NO: 9
ggtgcgagctgcagaatcacgagctgaccctgtggaacgaggcccgcaagctgaaccccaacgccatcgcctcggccaccgtggg
ccggcgggtgagcgcgcggatgctcggcgacgtgatggccgtctccacgtgcgtgccggtcgccgcggacaacgtgatcgtccaa
aactcgatgcgcatcagctcgcggcccggggcctgctacagccgccccctggtcagctttcggtacgaagaccagggcccgttggt
cgaggggcagctgggggagaacaacgagctgcggctgacgcgcgatgcgatcgagccgtgcaccgtgggacaccggcgctactt
caccttcggcgggggctacgtgtacttcgaggagtacgcgtactcccaccagctgagccgcgccgacatcaccaccgtcagcacctt
catcgacctcaacatcaccatgctggaggatcacgagtttgtccccctggaggtgtacacccgccacgagatcaaggacagcggcct
gctggactacacggaggtccagcgccgcaaccagctgcacgacctgcgcttcgccgacategacacggtcatccacgccgacgcc
aacgccgccatgttcgcgggcctgggcgcgttcttcgaggggatgggcgacctggggcgcgcggtcggcaaggtggtgatgggca
tcgtgggcggcgtggtatcggccgtgtcgggcgtgtcctccttcatgtccaacccctttggggcgctggccgtgggtctgttggtcctg
gccggcctggcggcggctttcttcgcctttcgctacgtcatgcggctgcagagcaaccccatgaaggccctgtacccgctaaccacca
aggagctcaagaaccccaccaacccggacgcgtccggggagggcgaggagggcggcgactttgacgaggccaagctagccga
ggcccgggagatgatacggtacatggccctggtgtctgccatggagcgcacggaacacaaggccaagaagaagggcacgagcgc
gctgctcagcgccaaggtcaccgacatggtcatgcgcaagcgccgcaacaccaactacacccaagttcccaacaaagacggtgac
gccgacgaggacgacctgtgacggggggtttgttgtaaataaaaaccacgggtgttaaaccgcatgtgcatcttttggtttgtttgtttgg
tacgccttttgtgtgtgtgggaagaaagaaaagggaacacataaactcccccgggtgtccgcggcctgtttcctctttcctttcccgtgac
aaaacggacccccttggtcagtgccgattccccccccacgccttcc
Homologyarm-2L
SEQ ID NO: 10
agcccgggccccccgcgggcggggcggcgcgcaaaaaaggcgggcggcggtccgggcggcgtgcgcgcgcgcggcgggcg
ttggggagcggggggaggagcggggggaggagcggggggaggagcggggggaggagcggggggaggagcggggggagg
agcggggggaggagcggggggaggagcggggggaggagcggggggaggagcggggggaggagcggggggaggagcgg
ggggaggagcggggggaggagcggggggaggagcggggggaggagcggggggaggagcggggggaggagcggaaaac
gggccccccccgaaacacaccccccgggggtcgcgcgcggccctttaaagcgcggcggcgcagcccgggccccccgcggccg
agacgagcgagttagacaggcaagcactactcgcctctgcacgcacatgcttgcctgtcaaactctaccaccccggcacgctctctgt
ctccatggcccgccgccgccgccatcgeggcccccgccgcccccggccgccegggcccacgggcgccgtcccaaccgcacagt
cccaggtaacctccacgcccaacteggaacccgcggtcaggagcgcgcccgcggccgccccgccgccgccccccgccagtggg
cccccgccttcttgttcgctgctgctgcgccagtgg
Homologyarm-2R
SEQ ID NO: 11
gacagccccccgcccgagccgggccagaggcceggcccaccgccgccgccccccgcccccggtccccaccgcccggcgcg
ggcccggggggcggggctaacccctcccaccccccctcacgccccttccgccttccgccgegcctcgccctccgcctgcgcgtcac
cgcagagcacctggcgcgcctgcgcctgcgacgcggggggggagggggcgccggagccccccgegacccccgegacccc
cgcgacccccgcgacccccgcgacccccgcgcgggtgcgcttctcgccccacgtccgggtgcgccacctggtggtctgggcctcg
gccgcccgcctggcgcgccgcggctcgtgggcccgcgagcgggccgaccgggctcggttccggcgccgggtggcggaggccg
aggcggtcatcgggccgtgcctggggcccgaggcccgtgcccgggccctggcccgcggagccggcccggcgaactcggtctaa
cgttacacccgaggcggcctgggtcttccgcggagctcccgggagctccgcaccaagccgctctccggagagacgatggcaggag
ccgcgcatatatacgctgggagccggcccgcccccgaggcgggcccgccctcggaggggggactggccaatcggggccgcc
agcgcggcggggcccggccaaccagcgtccgccgagtcttcggggcccggcccactggggggagttaccgcccagtgggccg
ggccgcccacttcccggtatggtaattaaaaacttacaagaggccttgttccgcttcccggtatggtaattagaaactcattaatgggcg
gccccggccgcccttcccgcttccggcaattcccgcggcccttaatgggcaaccceggtattccccgcctcccgcgccgcgcgtaac
cactcccctggggttccgggttatgctaattgcttttttgg
Homologyarm-3L
SEQ ID NO: 12
cgttcggacgtcttagaatcatggcggttttctatgccgacatcggttttctcccccgcaataagacacgatgcgataaaatctgtttgtga
aatttattaagggtacaaattgccctagcacaggggggggttagggccgggtccccacacccaaacgcaccaaacagatgcaggc
agtgggtcgagtacagccccgcgtacgaacacgtcgatgcgtgtgtcagacagcaccagaaagcacaggccatcaacaggtcgtg
catatgtcggtgggtttggacgcggggggccatggtggtgataaagttaatggccgccgtccgccagggccacaggggcgacgtct
cttggttggcccggagccactgggtgtggaccagccgcgcgtggcggcccaacatggcccctgtagccgggggcgggggatcgc
gcacgtttgcagcgcacatgcgagacacctcgaccacggttcggaagaaggcccggtggtccgcgggcaacatcaccaggtgcgc
aagcgcccgggcgtccagagggtagagccctgagtcatccgaggttggctcatcgcccgggtcatgccgcaagtgcgtgtgggttg
ggcttccggtgggcgggacgcgaaccgcggtgtggagccctacgcgggcccgagcgtacgctccatcttgtggggagaaggggt
ctgggctcgccaggggggcatacttgcccgggctatacagacccgcgagccgtacgtggttcgcggggggtgcgtggggtccggg
gctcccggggaggccggggctcccggggttgtcgtggatccctggggtcacgcggtaccctggggtctctgggagctcgcggtact
ctgggttccctaggttctcggggtggtcgcggaacccggggctcccggggaacacgcggtgtcctggggattgttggcggtcggac
ggcttcagatggcttcgagatcgtagtgtccgcaccgactcgtagtagacccgaatctccacattgccccgccgcttgatcattatcacc
ccgttgcgggggtccggagatcatgcgcgggtgtcctcgaggtgcgtgaacacctctggggtgcatgccggcggacggcacgcctt
ttaagtaaacatctgggtcgcccggcccaactggggccgggggttgggtctggctcatctcgagagccacggggggaaccaccctc
cgcccagaaacttgggcgatggtcgtaccc
Homologyarm-3R
SEQ ID NO: 13
gcctcgacgaggacgttcctcctgcgggaaggcacgaacgcgggtgagccccctcctccgcccccgcgtcccccctcctccgccc
ccgcgtcccccctcctccgcccccggtcccccctcctccgcccccgcgtcccccctcctccgcccccgcgtcccccctcctccgcc
cccgcgtcccccctcctccacccccgcgtccccccctcctccgcccacccaaggtgcttacccgtgcacaaaggcggaccggtggg
tttctgtcgtcggaggcccccggggtgcgtcccctgtgtttcgtgggggggggggggtctttccgcgtgtccctttccgatgcgatc
ccgatcccgagccggggcgtcgcgatgccgacgccgtccgctccgacggccctctgcgagtcccgctcccggtccgcgtgctccg
cagccgctcccgtcgttcgtggccggcgccgtctgcgggcgtcggtcgcgccgggcctttatgtgcgccggagagacccgcccccc
gccgcccgggcccgcccccggggccggcgcggagtcgggcacggcgccagtgctcgcacttcgccctaataatatatatatattgg
gacgaagtgcgaacgcttcgcgttctcacttcttttacccggcggccccgcccccttggggcggtcccgcccgccggccaatggggg
ggcggcaagggggcggcccttgggccgcccgccgtcccgttggtcccaacgtccggggggggaccgggggcccggggac
ggccaacgggcgcgcggggctcgtatctcattaccgccgaaccgggaagtcggggcccgggccccgcccccggcccgttcctcgt
tagcatgcggaacggaagcggaaaccaccggatcgggcggtaatgagatgccatgcggggggggcgcgggcccacccgccct
cgcgccccgcccatggcagatggcgcggatgggggggccgggggttcgaccaacgggccgcggccacgggcccccggcgtg
ccggcgtcggggggggtcgtgcataatggaattccgttcggggcgggcccgcctggggggggggggccggcggcctccgct
gctcctccttcccgccggcccctgggactatatgagcccgaggacgccccgatcgtccacacggagcgcggctgccgacacggatc
cacgacccgacgcgggaccgccagagacagaccgtcagacgctcg
Homologyarm-4L
SEQ ID NO: 14
gttactaaacacgaccctgaccgtcaagcgcggggcggcggcgtcccactctagaatcggttgggaccgcttcgtgggcggagttat
ccgccggttggccgcgcgccgccccggcctggtgtttatgctctggggcgcacatgcccagaatgccatcaggccggaccctcggg
tccattgcgtcctcaagttttcgcacccgtcgcccctctccaaggttccgttcggaacatgccagcatttcctcgtggcgaatcgatatctc
gagacccggtcgatttcacccatcgactggtcggtttgaaaggcatcgacgtccggggttttcgtctgtgggggcttttgggtatttccga
tgaataaagacggttaatggttaaacctctggtctcatacgggtcggtgatgtcgggcgtcgggggagagggagttccctctgcgcttg
cgattctagcctcgtggggctggacgttcgacacgccaaaccacgagtcagggatatcgccagatacgactcccgcagattccattcg
gggggccgctgtggcctcacctgaccaacctttacacgggggcccggaacgggaggccacagcgccgtctttctccccaacgcgc
gcggatgacggcccgccctgtaccgacgggccctacgtgacgtttgataccctgtttatggtgtcgtcgatcgacgaattagggcgtc
gccagctcacggacaccatccgcaaggacctgcggttgtcgctggccaagtttagcattgcgtgcaccaagacctcctcgttttcggg
aaacgccccgcgccaccacagacgcggggcgttccagcgcggcacggggcgccgcgcagcaacaaaagccttcagatgtttgt
gttgtgcaaacgcacccacgccgctcgagtgcgagagcagcttcgggtcgttattcagtcccgcaagccgcgcaagtattacacgcg
atcttcggacgggcggctctgccccgccgtccccgtgttcgtccacgagttcgtctcgtccgagccaatgcgcctccaccgagataac
gtcatgctggcctcgggggccgagtaaccgcccccccgcgccaccctcactgcccgtcgc
Homologyarm-4R
SEQ ID NO: 15
Gcgtgtttgatgttaataaataacgcataaatttggctggttgtttgttgtctttaatggaccgcccgcagggggggtggcatttcagtgtc
gggtgacgagcgcgatccggccgggatcctaggaccccaaaagtttgtctgcgtattccagggcggggctcagttgaatctcccgca
gcacctctaccagcaggtccgcggtgggctggagaaactcggccgtcccggggcaggcggtcgtcgggggggaggcgcggcg
cccaccccgtgtgccgcgcctggcgtctcctctgggggcgacccgtaaatggttgcagtgatgtaaatggtgtccgcggtccagacc
acggtcaaaatgccggccgtggcgctccgggcgctttcgccgcgcgaggagctgacccaggagtcgaacggatacgcgtacatat
gggcgtcccacccgcgttcgagcttctggttgctgtcccggcctataaagcggtaggcacaaaattcggcgcgacagtcgataatcac
caacagcccaatgggggtgtgttggataacaacgcctccgcgcggcaggcggtcctggcgctcccggccccgtaccatgatcgcgc
gggtgccgtactcaaaaacatgcaccacctgcgcggcgtcgggcagtgcgctggtcagcgaggccctggcgtggcataggctatac
gcgatggtcgtctgtggattggacatctcgcggtgggtagtgagtcccccgggccgggttcggtggaactgtaaggggacggcggg
ttaatatacaatgaccacgttcggatcgcgcagagccgatagtatgtgcttactaatgacgtcatcgcgctcgtggcgctcccggagcg
gatttaagttcatgcgaaggaattcggaggaggtggtgcgggacatggccacgtacgcgctgttgaggcgcaggttgccgggcgtaa
agcagatggcgaccttgtccaggctaaggccctgggagcgcgtgatggtcatggcaagcttggagctgatgccgt
The amino acid sequence of IL-12 P40
SEQ ID NO: 16
MCHQQLVISWFSLVFLASPLVAIWELKKDVYVVELDWYPDAPGEMVVLTCDTPEED
GITWTLDQSSEVLGSGKTLTIQVKEFGDAGQYTCHKGGEVLSHSLLLLHKKEDGIWS
TDILKDQKEPKNKTFLRCEAKNYSGRFTCWWLTTISTDLTFSVKSSRGSSDPQGVTCG
AATLSAERVRGDNKEYEYSVECQEDSACPAAEESLPIEVMVDAVHKLKYENYTSSFFI
RDIIKPDPPKNLQLKPLKNSRQVEVSWEYPDTWSTPHSYFSLTFCVQVQGKSKREKK
DRVFTDKTSATVICRKNASISVRAQDRYYSSSWSEWASVPCS
The amino acid sequence of IL-12 P35
SEQ ID NO: 17
MATTMWPPGSASQPPPSPAAATGLHPAARPVSLQCRLSMCPARSLLLVATLVLLDHLS
LARNLPVATPDPGMFPCLHHSQNLLRAVSNMLQKARQTLEFYPCTSEEIDHEDITKDK
TSTVEACLPLELTKNESCLNSRETSFITNGSCLASRKTSFMMALCLSSIYEDLKMYQV
EFKTMNAKLLMDPKRQIFLDQNMLAVIDELMQALNFNSETVPQKSSLEEPDFYKTKI
KLCILLHAFRIRAVTIDRVMSYLNAS
The amino acid sequence of OX40L
SEQ ID NO: 18
MLPLLLGLYGSVTSALKAAELYPRAHRIMYRMQLLSCIALSLALVTNSDQDKIEALSS
KVQQLERSIGLKDLAMADLEQKVLEMEASTQVSHRYPRIQSIKVQFTEYKKEKGFILT
SQKEDEIMKVQNNSVIINCDGFYLISLKGYFSQEVNISLHYQKDEEPLFQLKKVRSVN
SLMVASLTYKDKVYLNVTTDNTSLDDFHVNGGELILIHQNPGEFCVLASVDYKDDD
DKGSTSGSGKPGSGEGSTKGENLYFQGDLNAVGQDTQEVIVVPHSLPFKVVVISAILA
LVVLTIISLIILIMLWQKKPR
The amino acid sequence of PD-1 scfv
SEQ ID NO: 19
MYRMQLLSCIALSLALVTNSQVQLVESGGGVVQPGRSLRLDCKASGITFSNSGMHW
VRQAPGKGLEWVAVIWYDGSKRYYADSVKGRFTISRDNSKNTLFLQMNSLRAEDTA
VYYCATNDDYWGQGTLVTVSSGGGGSGGGGSGGGGSEIVLTQSPATLSLSPGERATL
SCRASQSVSSYLAWYQQKPGQAPRLLIYDASNRATGIPARFSGSGSGTDFTLTISSLEP
EDFAVYYCQQSSNWPRTFGQGTKVE
VH amino acid sequence of PD-1 scFv
SEQ ID NO: 20
QVQLVESGGGVVQPGRSLRLDCKASGITFSNSGMHWVRQAPGKGLEWVAVIWYDG
SKRYYADSVKGRFTISRDNSKNTLFLQMNSLRAEDTAVYYCATNDDYWGQGTLVTV
SS
VL amino acid sequence of PD-1 scFv
SEQ ID NO: 21
EIVLTQSPATLSLSPGERATLSCRASQSVSSYLAWYQQKPGQAPRLLIYDASNRATGIP
ARFSGSGSGTDFTLTISSLEPEDFAVYYCQQSSNWPRTFGQGTKVE
PD-1 scFv VH CDR1:
SEQ ID NO: 22
NSGMH
PD-1 scFv VH CDR2:
SEQ ID NO: 23
VIWYDGSKRYYADSVKG
PD-1 scFv VH CDR3:
SEQ ID NO: 24
NDDY
PD-1 scFv VL CDR1:
SEQ ID NO: 25
RASQSVSSYLA
PD-1 scFv VL CDR2:
SEQ ID NO: 26
DASNRAT
PD-1 scFv VL CDR3:
SEQ ID NO: 27
QQSSNWPRT
Signal peptide:
SEQ ID NO: 28
MYRMQLLSCIALSLALVINS
Primer HLA-A-F:
SEQ ID NO: 29
TGTTCTAAAGTCCGCACGC
Primer HLA-A-R:
SEQ ID NO: 30
TACCTCATGGAGTGGGAGC
Primer HLA-C-F:
SEQ ID NO: 31
CAGTTCGTGCGGTTCGACAG
Primer HLA-C-R:
SEQ ID NO: 32
GCCTGGCGCTTGTACTTCTG
Primer HLA-DRB1-F:
SEQ ID NO: 33
TGGTCCTGTCCTGTTCTCCA
Primer HLA-DRB1-R:
SEQ ID NO: 34
AGAAACGTGGTCTGGTGTCC
Primer PD-L1-F:
SEQ ID NO: 35
TTGCTGAACGCCCCATACAA
Primer PD-L1-R:
SEQ ID NO: 36
TCCAGATGACTTCGGCCTTG
Primer CD80-F:
SEQ ID NO: 37
CTCAGAAGTGGAGTCTTACCCTG
Primer CD80-R:
SEQ ID NO: 38
TGTTCCTGGGTCTCCAAAGG
Primer CD83-F:
SEQ ID NO: 39
CGCCCACTTGTCCCACTATC
Primer CD83-R:
SEQ ID NO: 40
CATTAGCCCATGCAACAGCC
Primer CD86-F:
SEQ ID NO: 41
TAGCACAGACACACGGATGAG
Primer CD86-R:
SEQ ID NO: 42
ACTGAAGTTAGCAGAGAGCAGG

1.-41. (canceled)

42. A two-factor recombinant HSV-1 oncolytic virus comprising in its genome two exogenous arming genes: a polynucleotide encoding a membrane-bound trimeric OX40L and a polynucleotide encoding an IL-12.

43. The two-factor recombinant HSV-1 oncolytic virus of claim 42, wherein the exogenous arming genes are inserted into the genomic loci of the recombinant oncolytic virus, selected from the group consisting of: the ICP34.5 locus, the intergenic region between UL3 and UL4, the intergenic region between UL50 and UL51, the intergenic region between US1 and US2, and the intergenic region between UL26 and UL27.

44. The two-factor recombinant HSV-1 oncolytic virus of claim 42, wherein the polynucleotide encoding the trimeric OX40L is inserted into one or two ICP34.5 loci of the virus genome.

45. The two-factor recombinant HSV-1 oncolytic virus of claim 42, wherein the polynucleotide encoding the IL-12 is inserted into one or two ICP34.5 loci of the virus genome.

46. The two-factor recombinant HSV-1 oncolytic virus of claim 42, wherein the polynucleotide encoding the trimetric OX40L is inserted into the two ICP34.5 loci of the virus genome and the polynucleotide encoding the IL-12 is inserted into the UL26-UL27 intergenic region of the virus genome.

47. The two-factor recombinant oncolytic virus of claim 42, wherein the recombinant oncolytic virus has a single-copy knockout or a double-copy knockout of the ICP34.5 gene and a knockout of the ICP47 gene in its genome.

48. The two-factor recombinant oncolytic virus of claim 42, wherein
the polynucleotide encoding trimeric OX40L encodes a trimeric OX40L polypeptide comprising from N- to C-terminus a trimerization domain, an extracellular domain of OX40L, and a transmembrane domain; the polypeptide comprises the amino acid sequence of SEQ ID NO: 18 or an amino acid sequence having at least 90%, 91%, 92%, 93%, 94%, 95%, 96%, 97%, 98%, or 99% identity thereto; and/or
the polynucleotide encoding IL-12 encodes an IL-12 dimeric protein comprising or composed of an IL-12α polypeptide and an IL-12β polypeptide; the IL-12α polypeptide comprises the amino acid sequence of SEQ ID NO: 17 or an amino acid sequence having at least 90%, 95%, 96%, 97%, 98%, or 99% identity thereto; and the IL-12β polypeptide comprises the amino acid sequence of SEQ ID NO: 16 or an amino acid sequence having at least 90%, 95%, 96%, 97%, 98%, or 99% identity thereto.

49. A recombinant oncolytic virus composition, comprising a recombinant HSV-1 oncolytic virus, wherein the at least one HSV-1 recombinant oncolytic virus comprises and expresses exogenous arming genes that encode a membrane-bound trimeric OX40L and an IL-12, and optionally a PD-1 inhibitor, upon infecting tumor cells.

50. The recombinant oncolytic virus composition of claim 49, wherein the recombinant oncolytic virus composition is a two-factor recombinant oncolytic virus composition providing a trimeric OX40L and an IL-12, which comprises a single recombinant HSV-1 oncolytic virus, wherein the recombinant HSV-1 oncolytic virus comprises, or only comprises, in its genome two exogenous arming genes: a polynucleotide encoding the trimeric OX40L and a polynucleotide encoding the IL-12.

51. The recombinant oncolytic virus composition of claim 49, wherein the exogenous arming genes are inserted into the genomic loci of the at least one recombinant oncolytic virus, selected from the group consisting of: the ICP34.5 locus, the intergenic region between UL3 and UL4, the intergenic region between UL50 and UL51, the intergenic region between US1 and US2, and the intergenic region between UL26 and UL27.

52. The recombinant oncolytic virus composition of claim 51, wherein the exogenous arming gene that encodes the trimeric OX40L is inserted into one or two ICP34.5 loci of the HSV-1 virus.

53. The recombinant oncolytic virus composition of claim 51, wherein the exogenous arming gene that encodes the IL-12 is inserted into one or two ICP34.5 loci of the HSV-1 virus.

54. The recombinant oncolytic virus composition of claim 49, wherein the recombinant HSV-1 oncolytic virus has a single-copy knockout or a double-copy knockout of the ICP34.5 gene and a knockout of the ICP47 gene in its genome.

55. The recombinant oncolytic virus composition of claim 49, wherein
the trimeric OX40L polypeptide has the amino acid sequence of SEQ ID NO: 18;
the IL-12 comprises an IL-12α having the amino acid sequence of SEQ ID NO: 17 and an IL-12β having the amino acid sequence of SEQ ID NO: 16; and/or
the PD-1 inhibitor is an anti-PD-1 single chain scFv antibody comprising the HCDR1-HCDR3 amino acid sequences of SEQ ID NOS: 22-24 and the LCDR1-LCDR3 amino acid sequences of SEQ ID NOS: 25-27, comprising the VH amino acid sequence of SEQ ID NO: 20 and the VL amino acid sequence of SEQ ID NO: 21, or the scFv antibody comprises or consists of the amino acid sequence of SEQ ID NO: 19.

56. The recombinant oncolytic virus composition of claim 49, wherein:
each of the at least one recombinant HSV-1 oncolytic virus comprises no more than 4, no more than 3, or no more than 2 exogenous arming genes in the viral genome; and
the recombinant oncolytic virus composition provides a total of no more than 6, no more than 4, no more than 3, or no more than 2 exogenous arming genes.

57. A method for treating a cancer in a subject, or for improving adoptive cell therapy in a cancer subject, the method comprising administering to the subject:
(a) a recombinant oncolytic virus composition of claim 49,
(b) a recombinant oncolytic virus composition of claim 49 and a PD-1 inhibitor, or
(c) (a) or (b) with an adoptive cell therapy composition,
wherein the adoptive cell therapy composition comprises tumor infiltrating lymphocytes (TILs),
wherein the TILs are derived from the same subject from whom the tumor cells originate.

58. The method of claim 57, wherein the recombinant oncolytic virus composition is administered intratumorally, and optionally, the adoptive cell therapy composition is administered intratumorally.

59. The method of claim 57, wherein the cancer is a solid tumor, head and neck cancer, oral cancer, gingival cancer, buccal cancer, tongue cancer, digestive system cancer, colorectal cancer, pancreatic cancer, glioblastoma, or melanoma, and metastases thereof,
or a squamous cell carcinoma or adenocarcinoma.

60. The method of claim 57, wherein the recombinant oncolytic virus composition is administered in an amount effective for converting tumor cells into antigen presenting cells (APCs) or enhancing the activation of tumor infiltrating lymphocytes (TIL cells) at the tumor site.

61. The method of claim 57, wherein the subject is a mammal or a human.
